(19) United States
(12) Patent Application Publication
(10) Pub. No.: US 2021/0147932 A1
(43) Pub. Date: May 20, 2021

(54) FLUORESCENT PROBES FOR QUANTIFICATION OF DNA DAMAGE AND REPAIR

(71) Applicant: The Board of Trustees of the Leland Stanford Junior University, Stanford, CA (US)

(72) Inventors: Eric T. Kool, Stanford, CA (US); David L. Wilson, Palo Alto, CA (US)

(21) Appl. No.: 17/088,119

(22) Filed: Nov. 3, 2020

Related U.S. Application Data
(60) Provisional application No. 62/936,055, filed on Nov. 15, 2019.

(51) Int. Cl. C12Q 1/6876; C07D 221/20; C07D 271/12; C07D 285/14; C07D 265/38; C07D 211/00

(52) U.S. Cl. CPC C12Q 1/6876; C07D 221/20; C07D 271/12; C12Q 2563/107; C07D 265/38; C07D 211/00; C12Q 2521/50; C07D 285/14

(57) ABSTRACT

Probes, methods and kits for detecting and measuring abasic (AP) sites in a nucleic acid are provided. Aspects of the methods include determining glycosylase enzyme activity. Further provided herein are methods of quantifying AP sites in genomic DNA, and quantifying the amount of DNA damage. The subject probes include a fluorophore linked to an alpha nucleophile that reacts with the AP site of the nucleic acid to produce a highly fluorescent conjugate.

### CROSS REFERENCE

This application claims benefit of U.S. Provisional Patent Application No. 62/936,055 filed Nov. 15, 2019. This application is incorporated herein by reference its entirety.

### FEDERALLY SPONSORED RESEARCH AND DEVELOPMENT

This invention was made with Government support under contract CA217809 awarded by the National Institutes of Health. The Government has certain rights in the invention.

### INTRODUCTION

The study of the mechanisms of DNA damage and repair is critically important to understanding the origin of diseases, such as cancer. DNA glycosylases are a class of DNA repair enzyme responsible for initiating base excision repair (BER). Enzymes of this broad class recognize damaged or mispaired DNA bases and hydrolyze the N-glyosidic bond between the targeted base and the sugar. The resulting hemiacetal abasic (AP) site created by base excision is then cleaved and ultimately filled in by downstream repair enzymes using the complementary strand to preserve the original genetic information. Most glycosylases can play a genoprotective role, preventing the accumulation of cytotoxic mutations in the genome. In addition to genoprotection, glycosylases can play a role in areas such as immune responses and epigenetics.

Given the central roles of DNA glycosylases in cancer biology and their potential therapeutic impact, the development of probes to measure their activities is of interest. Conventional biochemical assays of DNA glycosylase activity require discontinuous, gel-based or radiation release assays, which are poorly suited to high throughput screens or assaying activity in biological contexts. Accordingly, improved methods and probes for the measurement of glycosylase activities, and quantification of DNA damage and repair in general are of interest.

### SUMMARY

Probes, methods and kits for detecting and measuring abasic (AP) sites in a nucleic acid are provided. Aspects of the methods include determining glycosylase enzyme activity. Further provided herein are methods of quantifying AP sites in genomic DNA as a measure of DNA damage.

The subject probes comprise a fluorophore linked to an alpha nucleophile that reacts with the AP site of the nucleic acid to produce a highly fluorescent conjugate. Aspects of the methods include contacting a nucleic acid with a subject probe under conditions for reaction of the alpha nucleophile of the probe with the AP sites of the nucleic acid, thereby producing a conjugate; and detecting a fluorescence response generated by the conjugate to determine the presence of one or more AP sites in the nucleic acid. In certain cases, the nucleic acid is DNA. This disclosure includes methods where the nucleic acid is DNA and the DNA is contacted with a glycosylase enzyme to generate DNA with AP sites, such that detecting the presence of one or more AP sites in the DNA is used to determine the activity of the glycosylase enzyme. In certain aspects, the nucleic acid is a purified genomic DNA and the method further comprises comparing the fluorescence response of the conjugate to a standard to quantify the prevalence of AP sites in the purified genomic DNA. In certain cases, where the nucleic acid is a purified genomic DNA, the method further includes a pretreating step where the DNA is contacted with a corresponding DNA repair enzyme before contacting the DNA with the probe. The number of AP sites in the pre-treated sample is then compared to the number of AP sites in an untreated DNA sample to quantify the amount of DNA damage. Also provided herein are kits including a subject probe and a DNA repair enzyme.

These and other advantages and features of the disclosure will become apparent to those persons skilled in the art upon reading the details of the probes and methods of use, which are more fully described below.

### BRIEF DESCRIPTION OF THE FIGURES

FIG. 1 illustrates the fluorescent response mechanism of an exemplary subject probe in measuring DNA glycosylase activity. Upon excision of a damaged DNA base by the glycosylase of interest, the resulting hemiacetal form of the DNA AP site reacts with the aminooxy linker of the probe (also referred to herein as a universal base excision reporter (UBER) probe) to yield a strongly fluorescent probe-DNA oxime conjugate. Prior to oxime formation with the DNA AP site, the probe is largely non-fluorescent.

FIG. 2A illustrates structures of exemplary probes and aminooxy linkers.

FIG. 2B provides emission spectra of oligonucleotide-probe conjugates compared to the emission spectra of the free probe at 2 μM concentration in 50 mM Tris buffer pH 7.0. Excitation was at 470 nm for Compound (1) (also referred to herein as CCVJ1) and 450 nm for Compounds 8 and 9 (also referred to herein as NP1 and NP2) and Compounds 4 and 6 (also referred to herein as BD1 and BD2).

FIG. 3 provides representative plots for calculations of k₂ for Compound (1) (CCVJ1) under various buffer conditions with oligo 15. Plots were fitted in OriginPro 8.5 according to the equation derived by Larsen et. al.⁵ for second-order rate constants of oxime formation. Apparent second-order rate constants were calculated as the average of three replicates.

FIG. 4 provides excitation and emission spectra of DNA/probe conjugates (5 μM) with Compounds (1), (8) and (6) (CCVJ1, NP1 and BD1) with oligo 15 in 50 mM Tris buffer at pH 7.0 (ionic strength adjusted to 100 mM with NaCl). Photograph taken on a gel illuminator with excitation at 365 nm (CCVJ1=5 μM, NP1 and BD1=10 μM)

FIG. 5A provides fluorescence real-times responses for oxime formation under varied conditions. The Time course of oxime formation between Compound (1) (CCVJ1) (5 μM) and AP-DNA (20 μM) under varied buffer conditions (50 mM) is shown. Ionic strength adjusted to 150 mM with NaCl

FIG. 5B provides Time course of Compounds (1)-(3) (also referred to as CCVJ1-3) (5 μM) with AP-DNA (20 μM) in 50 mM Tris pH 7.0 buffer. Ionic strength adjusted to 150 mM with NaCl.

FIG. 6 provides effect of adding exemplary linker NH₂CH₂CH₂ONH₂ to the reaction of Compound (1) (CCVJ1) (5 μM) with AP site containing DNA (20 μM) in buffer. The plot shows that the linker does not significantly compete with CCVJ1 for oxime formation below concentrations of 500 μM.

FIG. 7 illustrates a comparison of relative fluorescence intensity of Compounds (1), (2) and (3) (CCVJ1-3) when bound to the AP site of Oligo 15 DNA.

FIG. 8 provides representative plot for the reaction of Compound (1) (CCVJ1) with single stranded DNA oligonucleotide 18.

FIG. 9 provides interaction between Compound (1) (CCVJ1) and DNA. Top graph shows Fluorescence response of CCVJ1 (5 μM) with 20 μM of undamaged double stranded DNA or a pseudo AP containing DNA strand compared to covalent linkage to a true AP site. The bottom graph shows Oxime formation between Compound (1) (CCVJ1) (5 μM) and AP site containing DNA (20 μM) with and without 20 μM pseudo AP containing hairpin. (Ex. 485, Em. 538).

FIG. 10, panels A-D illustrate effects of structural context on Compound (1) (CCVJ1 probe). Panels A-B, base identities of X and Y are listed on x-axis as neighboring pairs XY. Panels C-D, base identity of Z is listed on x-axis. Panels A and C show relative reaction rates between CCVJ1 (5 μM) and AP-DNA (20 μM) with varied neighboring bases (panel A) or opposing bases (panel C). Panels B and D show maximum fluorescence signal observed between CCVJ1 (5 μM) and AP-DNA (20 μM) with varied neighboring bases (panel B) or opposing bases (panel D). All values are based on average of duplicate runs.

FIG. 11 provides comparison of exemplary oligonucleotide-probe conjugate emission spectra (Ex 470 nm) at 2 μM when the abasic site is paired with adenine or cytosine. The cytosine base yields a 2-fold increase in overall fluorescence. A very slight (4 nm) red-shift in emission maxima was observed when the AP site is base paired against a pyrimidine.

FIG. 12 illustrates a computational model of Compound (1) (CCVJ1) bound to the AP site of duplex DNA, created using the Drew-Dickerson dodecamer as a starting structure.

FIG. 13 provides fluorescence response of 5 μM Compound (1) (CCVJ1) with 500 μM of various biologically relevant carbonyl species after 1 h incubation in buffer at 37° C. The absence of any fluorescence response suggests that oxime formation with small molecules does not appreciably constrain bond rotation. (Ex. 485, Em. 538)

FIG. 14 provides effect of competing aldehydes/ketones on maximum fluorescence signal. Compound (1) (CCVJ1 (5 μM) was reacted with AP DNA (20 μM) in the presence of increasing amounts of deoxyribose or pyruvate. Maximum fluorescence values were observed after 1 hr and compared to the control with no competing aldehyde/ketone. Given the generally slow rate known for oxime formation (e.g., about 0.001-0.1 M^{-1s-1}), such rapid rate acceleration was quite surprising. Furthermore, as disclosed herein, the linker length was found to significantly impact on the rate of oxime formation with shorter linkers favoring more rapid oxime formation.

FIG. 15, panels A-G provides quantitative coupled assays using Compound (1) (CCJV1). Shown are data from reaction of lesion containing substrates with UNG and SMUG1 (5 nM) (panel A), MPG (100 nM) (panel B), NTH1 (500 nM) (panel C), OGG1 (100 nM) in real-time (panel D) with lesion containing DNA (2 μM) and CCVJ1 (20 μM) (panels A, B and D) or lesion containing DNA (60 μM) and CCVJ1 (20 μM) (panel D). Panel E shows sequences of lesion containing hairpin substrates. Panel F shows an IC₅₀ curve of UGI with UNG fitted to the Boltzmann equation. Panel G shows real-time fluorescence traces of UNG with increasing concentrations of UGI (0.3 to 30 nM). Delay time tₛₛ of 25 minutes indicated with dotted line.

FIG. 16 provides time course of NTH1 (500 nM) with 5-hydroxycytosine containing DNA (60 μM) and Compound (1) (CCVJ1) (20 μM). After 40 minutes, an additional aliquot of NTH1 was added (250 nM) which did not yield any significant increase in fluorescence. Following a 10-minute incubation period, additional substrate was then added (20 μM) which yielded an increase in fluorescence.

FIG. 17 provides measuring real-time MPG-mediated base excision of alkylated bases in calf thymus DNA (0.1 mg/mL) by 100 nM MPG enzyme in the presence of Compound (1) (CCVJ1) (20 μM).

FIG. 18A provides measurement of OGG1 activity (100 nM) on oxidized calf thymus DNA (ctDNA) generated in situ. DNA was oxidized by treating ctDNA (0.1 mg/mL) with increasing amounts of Fenton's reagent (Fe/H₂O₂).

FIG. 18B provides fluorescence signal observed by the repair of alkylated ctDNA with MPG and demonstrates a linear relationship between final fluorescence signal and amount of DMS used to treat ctDNA from.

FIG. 18C provides pre-incubation of alkylated ctDNA with CCVJ1 prior to addition of MPG.

FIG. 19, panels A-B illustrates applications of an exemplary probe for measuring multiple repair activities in cell lysates. Panel A shows fluorescence time-course of 25 μM of Compound (1) (CCVJ1) with HeLa cell lysate (0.2 mg/mL) and 5 μM of UNG hairpin substrate. The inhibitor UGI was used at a concentration of 1 U/mL to completely abolish UNG activity. Inset shows close overlap of the control and UGI treated lysates. Panel B shows quantification of UNG activity in actively replicating HeLa cells and G0/G1 cell cycle arrested HeLa cells. Activity was quantified by measuring initial rate velocity using 25 μM CCVJ1 with lysate (0.2 mg/mL) and 5 μM of UNG hairpin substrate.

FIG. 20 provides fluorescence measurement of MCF7 cell lysates (0.2 mg/mL) with 25 μM Compound (1) (CCVJ1) following a 4-hour incubation with 5 μM of an OGG1 hairpin substrate. The inhibitor SU0268 was used to abrogate OGG1 activity. Error bars represent standard deviation of three replicates.

FIG. 21 provides measurement of OGG1 activity in HeLa cells vs activity in MCF7 cells. Lysates (0.2 mg/mL) were added to buffer containing 5 μM of either oligo 18 (non-lesion control substrate) or oligo 22 (OGG1 substrate) with 25 μM Compound (1) (CCVJ1) and incubated for 4 hrs. (Ex. 485, Em. 538)

FIG. 22 provides measurement of UNG activity in HeLa cells vs activity in MCF7 cells. Lysates (0.2 mg/mL) were added to buffer containing 5 μM of either oligo 18 (non-lesion control substrate) or oligo 15 (UNG substrate) with 25 μM Compound (1) (CCVJ1) and incubated for 4 h. (Ex. 485, Em. 538).

FIG. 23 provides time course of AP-site oxime formation of Compound (8) (NP1) and Compound (6) (BD1) (5 μM) with Oligo 15 (20 μM) in buffer at 37° C. (Ex. 485, Em. 538)

FIG. 24 provides normalized absorption spectra of free Compound (1) (CCJV1 probe) and AP site bound probe in buffer. Oxime formation with the AP site red shifts absorption from a λₘₐₓ of 454 nm in the unbound probe to a λₘₐₓ of 468 nm in the bound probe. The extinction coefficient calculated for the unbound probe is 30,400 M⁻¹·cm⁻¹ and 28,700 M⁻¹·cm⁻¹ for the bound probe.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a dye” refers to one or more dyes, i.e., a single dye and multiple dyes. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “molecular rotor” refers collectively to a group of fluorescent compounds that possess the ability to undergo twisted intramolecular charge transfer (TICT). Molecular rotors include an electron-donating unit, an electron-accepting unit and a π-conjugated linking moiety which allows electron transfer to occur in the planar conformation.

The term “alpha nucleophile” refers to a nucleophile bearing an unshared pair of electrons on an atom adjacent to the nucleophilic site. Alpha nucleophiles, include, but is not limited to, aminooxy moieties, hydrazine moieties, hydrazide moieties and peroxide moieties.

As used herein the term “isolated,” refers to an moiety of interest that is at least 60% free, at least 75% free, at least 90% free, at least 95% free, at least 98% free, and even at least 99% free from other components with which the moiety is associated with prior to purification.

A “plurality” contains at least 2 members. In certain cases, a plurality may have 5 or more, such as 6 or more, 7 or more, 8 or more, 9 or more, 10 or more, 20 or more, 30 or more, 40 or more, 50 or more, 60 or more, 70 or more, 80 or more, 90 or more, 100 or more, 300 or more, 1000 or more, 3000 or more, 10,000 or more, 100,000 or more members.

Numeric ranges are inclusive of the numbers defining the range.

The methods described herein include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining,” “measuring,” and “assessing,” and “assaying” are used interchangeably and include both quantitative and qualitative determinations.

The term “separating”, as used herein, refers to physical separation of two elements (e.g., by size or affinity, etc.) as well as degradation of one element, leaving the other intact.

The term “linker” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some cases, the linker is a branching linker that refers to a linking moiety that connects three or more groups. In certain cases, one, two, three, four or five or more carbon atoms of a linker backbone may be optionally substituted with a sulfur, nitrogen or oxygen heteroatom. In some cases, the linker backbone includes a linking functional group, such as an ether, thioether, amino, amide, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

The terms “polyethylene oxide”, “PEO”, “polyethylene glycol” and “PEG” are used interchangeably and refer to a polymeric group including a chain described by the formula —(CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use in the subject probes include those PEGs described by S. Zalipsky in “Functionalized poly(ethylene glycol) for preparation of biologically relevant conjugates”, Bioconjugate Chemistry 1995, 6 (2), 150-165; and by Zhu et al in “Water-Soluble Conjugated Polymers for Imaging, Diagnosis, and Therapy”, Chem. Rev., 2012, 112 (8), pp 4687-4735.

The term “alkyl” by itself or as part of another substituent refers to a saturated branched or straight-chain monovalent hydrocarbon radical derived by the removal of one hydrogen atom from a single carbon atom of a parent alkane. Alkyl groups of interest include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group includes from 1 to 20 carbon atoms. In some embodiments, an alkyl group includes from 1 to 10 carbon atoms. In certain embodiments, a lower alkyl group includes from 1 to 6 carbon atoms, such as from 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Amino” refers to the group —NH₂. The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aryl” by itself or as part of another substituent refers to a monovalent aromatic hydrocarbon radical derived by the removal of one hydrogen atom from a single carbon atom of an aromatic ring system. Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

“Substituted aryl”, unless otherwise constrained by the definition for the aryl substituent, refers to an aryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂— alkyl, —SO₂-substituted alkyl, —SO₂-aryl, —SO₂-heteroaryl and trihalomethyl.

“Heteroaryl” by itself or as part of another substituent, refers to a monovalent heteroaromatic radical derived by the removal of one hydrogen atom from a single atom of a heteroaromatic ring system. Heteroaryl groups of interest include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, triazole, benzotriazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Substituted heteroaryl”, unless otherwise constrained by the definition for the substituent, refers to an heteroaryl group substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂— alkyl, —SO₂-substituted alkyl, —SO₂-aryl, —SO₂-heteroaryl and trihalomethyl.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like. “Substituted alkylene” refers to an alkylene group having from 1 to 3 hydrogens replaced with substituents as described for carbons in the definition of “substituted” below.

“Substituted” refers to a group in which one or more hydrogen atoms are independently replaced with the same or different substituent(s). Substituents of interest include, but are not limited to, alkylenedioxy (such as methylenedioxy), -M, —R⁶⁰, —O—, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂O—, —S(O)₂OH, —S(O)₂R⁶⁰, —OS(O)₂O—, —OS(O)₂R⁶⁰, —P(O)(O⁻)₂, —P(O)(OR⁶⁰)(O⁻), —O P(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶4 and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O—. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰,

—OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group. When the group being substituted is an aryl or heteroaryl group, the substituent(s) (e.g., as described herein) may be referred to as “aryl substituent(s)”.

It is understood that in all substituted groups defined above, derivatives arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be metabolized into a pharmaceutically active derivative.

Unless otherwise specified, reference to an atom is meant to include isotopes of that atom. For example, reference to H is meant to include 1H, 2H (i.e., D) and 3H (i.e., T), and reference to C is meant to include ¹²C and all isotopes of carbon (such as ¹³C).

Other definitions of terms may appear throughout the specification.

### DETAILED DESCRIPTION

Probes, methods and kits for detecting and measuring abasic (AP) sites in a nucleic acid are provided. Aspects of the methods include determining glycosylase enzyme activity. Further provided herein are methods of quantifying AP sites in genomic DNA, and quantifying the amount of DNA damage. The subject probes include a fluorophore linked to an alpha nucleophile that reacts with the AP site of the nucleic acid to produce a highly fluorescent conjugate. Aspects of the methods include contacting the nucleic acid with a subject probe under conditions for reaction of the alpha nucleophile of the probe with the AP sites of the nucleic acid thereby producing a conjugate; and detecting a fluorescence response generated by the conjugate to determine the presence of one or more AP sites in the nucleic acid. In certain cases, the nucleic acid is DNA. This disclosure includes methods where the nucleic acid is DNA and the DNA is contacted with a glycosylase enzyme to generate DNA with AP sites, such that the presence of one or more AP sites in the DNA determines the activity of the glycosylase enzyme. In certain aspects, the nucleic acid is a purified genomic DNA and the method further comprises comparing the fluorescence response of the conjugate to a standard to quantify the prevalence of AP sites in the purified genomic DNA. In certain cases, where the nucleic acid is a purified genomic DNA, the method further includes a pretreating step where the DNA is contacted with a corresponding DNA repair enzyme before contacting the DNA with the probe. The number of AP sites in the pre-treated sample is then compared to the number of AP sites in an untreated DNA sample to quantify the amount of DNA damage. Also provided herein are kits including a subject probe and a DNA repair enzyme.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Probes

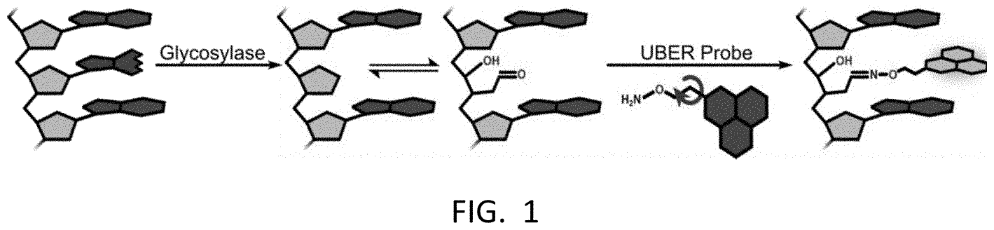

Aspects of the invention include probes for use in the methods described herein. The subject probes include a fluorophore linked to an alpha nucleophile that reacts with the AP site of the nucleic acid to produce a highly fluorescent conjugate. In some cases, the alpha nucleophile is an aminooxy group. In certain other cases, the alpha nucleophile is a hydrazine. In yet other cases, the alpha nucleophile is a hydrazide. In some cases, the fluorophore is a twisted internal charge transfer (TICT) compound. In some cases, the fluorophore is a molecular rotor. In both TICT and molecular rotor probes, a fluorophore that experiences non-radiative relaxation through bond rotation can be conjugated to a reactive functionality that targets the probe to a biomolecule of interest (e.g., DNA). Upon binding to the target biomolecule, bond rotation within the probe can become constrained, resulting in a significant fluorescence increase (e.g., as compared to the free probe). For example, FIG. 1 illustrates the fluorescence response mechanism of an exemplary subject probe in measuring DNA glycosylase activity. Upon excision of a damaged DNA base by the glycosylase of interest, the resulting hemiacetal form of the DNA AP site reacts with the aminooxy linker of the probe (also referred to herein as a universal base excision reporter (UBER) probe) to yield a strongly fluorescent probe-DNA oxime conjugate. Prior to oxime formation with the DNA AP site, the probe is largely non-fluorescent. Without being bound to any particular theory, this may be due to free bond rotation about the linker attachment site in the free probe. By contrast, the neighboring bases of the DNA duplex constrain bond rotation in the probe-DNA conjugate, yielding a fluorescence response. In some cases, reaction of the probe can be highly specific for the AP site of DNA over small-molecule aldehydes and ketones.

In one embodiment, there is provided a probe of formula (I):

A-L-Y
wherein:
A is a fluorophore;
L is a linker or a bond; and
Y is an alpha nucleophile.

In some instances of a probe of formula (I), Y is an alpha nucleophile selected from an aminooxy, a hydrazine and a hydrazide. Accordingly, in one embodiment, the probe of formula (I) is described by formula (IA):

A-L-ONH₂ (IA)
wherein:
A is a fluorophore; and
L is a linker or a bond.

In another embodiment, the probe of formula (I) is described by the formula (IB):

A-L-NR^{a}NH₂ (IB)
wherein:
A is a fluorophore;
L is a linker or a bond; and
R^{a} is selected from hydrogen, alkyl or substituted alkyl.

In another embodiment, the probe is of the formula (IC):

A-L-C(O)NR^{a}NH₂ (IC)

wherein:
A is a fluorophore;
L is a linker or a bond; and
R^{a} is selected from hydrogen, alkyl or substituted alkyl.

In some embodiments of a probe of formula (I), the fluorophore is a twisted intramolecular charge transfer (TICT) compound or a molecular rotor. Any convenient TICT compound or molecular rotor can find use as a fluorophore in the subject probes. In certain cases, the fluorophore is a TICT compound or a molecular rotor described in Yu, W.-T. et al. Protein Sensing in Living Cells by Molecular Rotor-Based Fluorescence-Switchable Chemical Probes. Chem. Sci. 2015, 7 (1), 301-307; and Liu, Y. et al. The Cation-π Interaction Enables a Halo-Tag Fluorogenic Probe for Fast No-Wash Live Cell Imaging and Gel-Free Protein Quantification. Biochemistry 2017, 56 (11), 1585-1595, the disclosures of which are incorporated herein by reference.

In certain embodiments of a probe of formula (I), the fluorophore A is selected from a naphthalimide compound, a naphthalic anhydride, a 9-(2-carboxy-2-cyanovinyl)julolidine (CCVJ) compound, a benzophenoxazinone (e.g., Nile Red), a benzoxadiazole, a styrylpyridinium, a stilbene, a cinnamonitrile compound, and a thiazole orange compound.

In certain cases, the fluorophore is a 1,8-napthalimide or a naphthalic anhydride compound based on the following core structures:

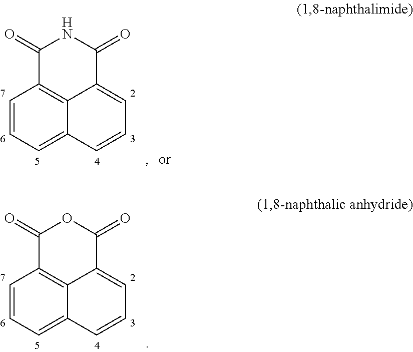 [SMILES: O=C1NC(=O)c2cccc3cccc1c23 ; O=C1OC(=O)c2cccc3cccc1c23]

In some cases, the 1,8-napthalimide or 1,8-naphthalic anhydride compound is further substituted at any feasible position. For example, in some cases, the core naphthalimide or naphthalic anhydride compound can be substituted at any one or more positions selected from 2, 3, 4, 5, 6, and 7 (e.g., as labeled above). In certain cases, the core naphthalimide or naphthalic anhydride compound is substituted at 1-2 positions selected from 2, 3, 4, 5, 6 and 7. In certain cases, the naphthalimide or naphthalic anhydride compound is substituted at the 2-position. In certain cases, the naphthalimide or naphthalic anhydride compound is substituted at the 3-position. In certain cases, the naphthalimide or naphthalic anhydride compound is substituted at the 4-position. In certain cases, the naphthalimide or naphthalic anhydride compound is substituted at the 5-position. In certain cases, the naphthalimide or naphthalic anhydride compound is substituted at the 6-position. In certain cases, the naphthalimide or naphthalic anhydride compound is substituted at the 7-position. In certain instances, the 1,8-napthalimide or 1,8-naphthalic anhydride compound is a 4-amino derivative. In certain cases, the nitrogen atom in 1,8-naphthalimide is further substituted (e.g., as described herein).

In certain cases, the fluorophore A is described by the formula (II-D):

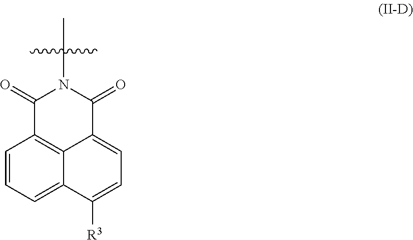

wherein:

R³ is selected from amino, substituted amino, alkyl, substituted alkyl, aryl, substituted aryl, acyl, carboxyl, substituted acyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-D), R³ is selected from an amino or a substituted amino group. In certain cases, R³ is a substituted amino group described by —N(R^{3a})₂, wherein each R^{3a} is independently selected from a C₍₁₋₆₎alkyl, or a substituted C₍₁₋₆₎. In some cases, one R^{3a} is hydrogen and the other R^{3a} is selected from C₍₁₋₆₎alkyl, or a substituted C₍₁₋₆₎ alkyl. In some cases, each R^{3a} group combine together with the nitrogen to which they are attached to form a 5 or 6-membered cyclic group. In some instances, each R^{3a} group combined with the nitrogen to which they are attached forms a 6-membered group selected from a piperidine, a piperazine, a pyridazine, a pyrazine, a triazine, a pyridine, a pyrimidine, a morpholine and a thiomorpholine. In some instances, each R^{3a} group combined with the nitrogen to which they are attached forms a 5-membered group selected from a pyrrolidine, a pyrroline, a pyrrole, an imidazolidine, a pyrazolidine, a pyrazole, an imidazole, a triazole, and a tetrazole.

While structures of formula (II-D) are drawn with substituent R³ at the 4-position of the 1,8-naphthalimide core, the substituent R³ (e.g., as described above) may also be present at any one or more of the 2, 3, 5, 6 or 7 positions of the 1,8-naphthalimide core.

In certain cases, the fluorophore A is described by the formula (II-E):

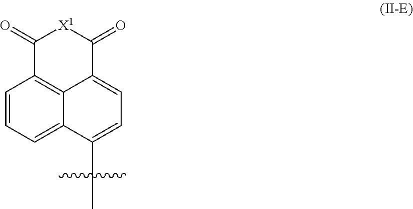

wherein:

X¹ is O or NR⁴; and

[symbol] represents the point of attachment to L.

In certain cases of the fluorophore of formula (II-E), X¹ is NR⁴, and R⁴ is selected from an alkyl or substituted alkyl group. In certain instances, R⁴ is selected from a C₍₁₋₆₎alkyl, or a substituted C₍₁₋₆₎ alkyl. In certain instances, R⁴ is a substituted C₍₁₋₆₎ alkyl of the formula —(CH₂)ₙR^{4a}, wherein R^{4a} is selected from acyl, carboxyl, amino, substituted amino, nitrile, and halogen. In some cases, R^{4a} is CO₂H. In certain other cases, R^{4a} is N(R^{3a})₂, where R^{3a} is as defined above. In some cases X¹ is O.

While structures of formula (II-E) are drawn with the point of attachment to L at the 4-position of the core, the point of attachment to L may also be present at the 2, 3, 5, 6 or 7 position of the naphthalimide or naphthalic anhydride core.

In certain cases, the fluorophore is a 9-(2-carboxy-2-cyanovinyl)julolidine (CCVJ) compound. CCVJ has the following core structure:

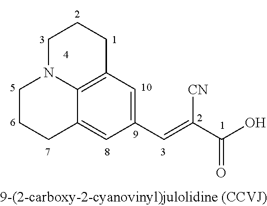 [SMILES: N#C/C(=C\c1cc2c3c(c1)CCCN3CCC2)C(=O)O]

In some cases, the CCVJ is further substituted at any feasible position. For example, in some cases, the core CCVJ cyclic structure can be substituted at any one or more positions selected from 1, 2, 3, 5, 6, 7, 8 and 10 (e.g., as labeled above). In certain cases, the core CCVJ compound is substituted at 1-2 positions selected from 1, 2, 3, 5, 6, 7, 8 and 10. In certain cases, the CCVJ compound is substituted at the 1-position. In certain cases, the CCVJ compound is substituted at the 2-position. In certain cases, the CCVJ compound is substituted at the 3-position. In certain cases, the CCVJ compound is substituted at the 5-position. In certain cases, the CCVJ compound is substituted at the 6-position. In certain cases, the CCVJ compound is substituted at the 7-position. In certain instances, the CCVJ compound is substituted at the 8-position. In certain cases, the CCVJ compound is substituted at the 10-position.

In certain cases, the fluorophore A is described by the formula (II-A):

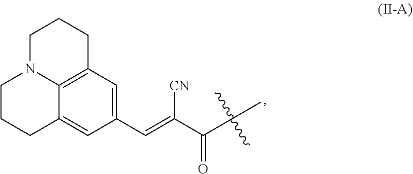

wherein [symbol] represents the point of attachment to L.

In certain cases, the fluorophore is a benzoxadiazole compound. In certain cases, the benzoxadiazole compound is a 2,1,3-benzoxadiazole compound or a 2,1,3-benzothiadiazole compound based on the following core structures:

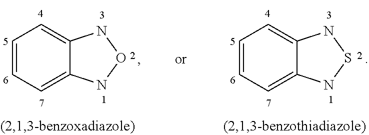 [SMILES: c1ccc2c(c1)NON2 ; c1ccc2c(c1)NSN2]

In some cases, the 2,1,3-benzoxadiazole or 2,1,3-benzothiadiazole compound is further substituted at any feasible position. For example, in some cases, the core benzoxadiazole or benzothiadiazole compound can be substituted at any one or more positions selected from 4, 5, 6 and 7 (e.g., as labeled above). In certain cases, the core benzoxadiazole or benzothiadiazole compound is substituted at 1-2 positions selected from 4, 5, 6 and 7. In certain cases, the benzoxadiazole or benzothiadiazole compound is substituted at the 4-position. In certain cases, the benzoxadiazole or benzothiadiazole compound is substituted at the 5-position. In certain cases, the benzoxadiazole or benzothiadiazole compound is substituted at the 6-position. In certain cases, the benzoxadiazole or benzothiadiazole compound is substituted at the 7-position. In certain instances, the benzoxadiazole or benzothiadiazole compound is substituted at the 4 and 7-positions (e.g., with a substituent as described herein).

In certain cases, the fluorophore A is described by the structure (II-B):

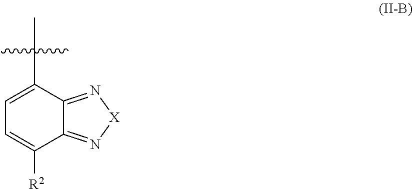

wherein:

X is O or S;

R² is selected from amino, substituted amino, alkyl, substituted alkyl, aryl, substituted aryl, acyl, carboxyl, substituted acyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-B), R² is selected from a sulfonamide, or substituted sulfonamide. In certain cases, R² is an amino or substituted amino. In certain cases, R² is C₍₁₋₆₎alkyl, or a substituted C₍₁₋₆₎ alkyl. In certain cases, R² is a sulfonamide group described by —SO₂N(R^{2a})₂, wherein each R^{2a} is independently selected from a C₍₁₋₆₎alkyl, or a substituted C₍₁₋₆₎ alkyl. In some cases, one R^{2a} is hydrogen and the other R^{2a} is selected from C₍₁₋₆₎alkyl, or a substituted C₍₁₋₆₎ alkyl. In some cases, each R^{2a} group combine together with the nitrogen to which they are attached to form a 5 or 6-membered cyclic group. In some instances, each R^{2a} group combined with the nitrogen to which they are attached forms a 6-membered group selected from a piperidine, a piperazine, a pyridazine, a pyrazine, a triazine, a pyridine, a pyrimidine, a morpholine and a thiomorpholine. In some instances, the R^{2a} groups together with the nitrogen to which they are attached form a 5-membered ring selected from a pyrrolidine, a pyrroline, a pyrrole, an imidazolidine, a pyrazolidine, a pyrazole, an imidazole, a triazole, and a tetrazole.

In certain cases of a fluorophore of formula (II-B) X is O. In other cases of a fluorophore of formula (II-B), X is S.

While structures of formula (II-B) are drawn with substituent R² at the 7-position of the benzoxadiazole or benzothiadiazole core, the substituent R² (e.g., as described above) may also be present at any one or more of the 4, 5 or 6 positions of the benzoxadiazole or benzothiadiazole core.

In certain cases, the fluorophore A is described by the structure (II-C):

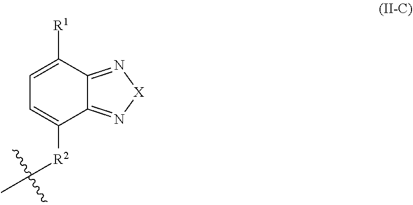

wherein:

X is O or S;

R¹ is selected from amino, substituted amino, alkyl, substituted alkyl, aryl, substituted aryl, acyl, carboxyl, substituted acyl, sulfonamide, substituted sulfonamide, nitro, nitrile, halogen, heteroaryl, substituted heteroaryl, heterocycle, and substituted heterocycle; R² is selected from sulfonyl, amino, thiol and oxy; and

[symbol] represents the point of attachment to L.

In certain cases of the fluorophore of formula (II-C), R² is selected from an sulfonyl. In certain cases, R² is an amino. In certain cases, R² is thiol. In certain cases, R² is oxy (e.g., to form a ketone functional group).

In certain cases of the fluorophore of formula (II-C), R¹ is selected from an amino or a substituted amino group. In certain cases, R¹ is a substituted amino group described by —N(R^{1a})₂, wherein each R^{1a} is independently selected from a C₍₁₋₆₎alkyl, or a substituted C₍₁₋₆₎ alkyl. In some cases, one R^{1a} is hydrogen and the other R^{1a} is selected from C₍₁₋₆₎alkyl, or a substituted C₍₁₋₆₎ alkyl. In some cases, each R^{1a} group combine together with the nitrogen to which they are attached to form a 5 or 6-membered cyclic group. In some instances, each R^{1a} group combined with the nitrogen to which they are attached forms a 6-membered group selected from a piperidine, a piperazine, a pyridazine, a pyrazine, a triazine, a pyridine, a pyrimidine, a morpholine and a thiomorpholine. In some instances, each R^{1a} group combined with the nitrogen to which they are attached forms a 5-membered group selected from a pyrrolidine, a pyrroline, a pyrrole, an imidazolidine, a pyrazolidine, a pyrazole, an imidazole, a triazole, and a tetrazole.

In certain cases of a fluorophore of formula (II-C) X is O. In other cases of a fluorophore of formula (II-C), X is S.

While structures of formula (II-C) are drawn with substituent R¹ at the 4-position of the benzoxadiazole or benzothiadiazole core, the substituent R¹ (e.g., as described above) may also be present at any one or more of the 5 or 6 positions of the benzoxadiazole or benzothiadiazole core.

In certain cases, the fluorophore is a benzophenoxazinone compound of the following core structure:

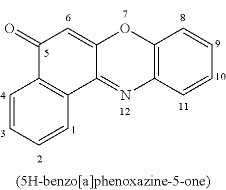 [SMILES: O=c1cc2oc3ccccc3nc-2c2ccccc12]

In some cases, the benzophenoxazinone is further substituted at any feasible position. For example, in some cases, the core benzophenoxazinone compound can be substituted at any one or more positions selected from 1, 2, 3, 4, 6, 8, 9, 10 and 11 (e.g., as labeled above). In certain cases, the core benzophenoxazinone compound is substituted at 1-2 positions selected from 1, 2, 3, 4, 6, 8, 9, 10 and 11. In certain cases, the benzophenoxazinone compound is substituted at the 1-position. In certain cases, the benzophenoxazinone compound is substituted at the 2-position. In certain cases, the benzophenoxazinone compound is substituted at the 3-position. In certain cases, the benzophenoxazinone compound is substituted at the 4-position. In certain cases, the benzophenoxazinone compound is substituted at the 8-position. In certain cases, the benzophenoxazinone compound is substituted at the 9-position. In certain instances, the benzophenoxazinone compound is substituted at the 10-position. In certain cases, the benzophenoxazinone compound is substituted at the 11-position. In certain cases, the benzophenoxazinone compound is substituted with an amino group at the 9-position. In certain cases, the benzophenoxazinone compound is 9-diethylamino-5-benzo[a]phenoxazinone (e.g., Nile Red, or Nile blue oxazone).

In certain cases, the fluorophore A is described by the formula (II-F):

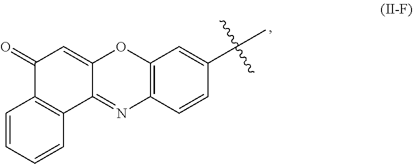

wherein [symbol] represents the point of attachment to L.

While structures of formula (II-F) are drawn with the point of attachment to L at the 9-position of the core, the point of attachment to L may also be present at any one of the 1, 2, 3, 4, 6, 8, 10 and 11 position of the benzophenoxazinone core. In addition, the compound of formula (II-F) may be further substituted with 1-3 substituents (e.g., with a substituent as described herein) at any feasible position.

In certain cases, the fluorophore is a styrylpyridium compound of the following core structure:

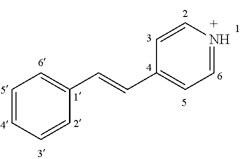 [SMILES: C(=C\c1ccncc1)/c1ccccc1]

In some cases, the styrylpyridinium is further substituted at any feasible position. For example, in some cases, the core styrylpyridinium compound can be substituted at any one or more positions selected from 1, 2, 3, 5, 6, 2′, 3′, 4′, 5′, and 6′ (e.g., as labeled above). In certain cases, the core styrylpyridinium compound is substituted at 1-2 positions selected from 1, 2, 3, 5, 6, 2′, 3′, 4′, 5′, and 6′. In certain cases, the styrylpyridinium compound is substituted at the 1-position. In certain cases, the styrylpyridinium compound is substituted at the 2-position. In certain cases, the styrylpyridinium compound is substituted at the 3-position. In certain cases, the styrylpyridinium compound is substituted at the 5-position. In certain cases, the styrylpyridinium compound is substituted at the 6-position. In certain cases, the styrylpyridinium compound is substituted at the 2′-position. In certain instances, the styrylpyridinium compound is substituted at the 3′-position. In certain cases, the styrylpyridinium compound is substituted at the 4′-position. In certain cases, the styrylpyridinium compound is substituted at the 5′-position. In certain cases, the styrylpyridinium compound is substituted at the 6′ position.

In certain cases, the fluorophore A is described by the formula (II-G):

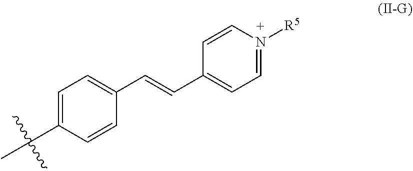

wherein:

R⁵ is selected from, alkyl, and substituted alkyl; and

[symbol] represents the point of attachment to L.

In certain cases of the fluorophore of formula (II-G), R⁵ is selected from a C₍₁₋₆₎alkyl, or a substituted C₍₁₋₆₎. In some cases, R⁵ is methyl. In some cases, R⁵ is ethyl.

While structures of formula (II-G) are drawn with the point of attachment to L at the 4′-position of the styrylpyridinium core, the point of attachment to L may also be present at any other position of the styrylpyridinium core.

In certain cases, the fluorophore A is described by the formula (II-H):

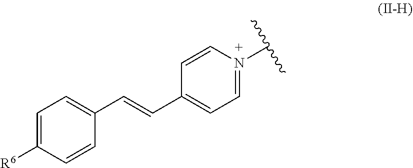

wherein:

R⁶ is selected from amino, substituted amino, alkyl, substituted alkyl, aryl, substituted aryl, acyl, carboxyl, substituted acyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-H), R⁶ is selected from an amino or a substituted amino group. In certain cases, R⁶ is a substituted amino group described by —N(R^{6a})₂, wherein each R^{6a} is independently selected from a C₍₁₋₆₎alkyl, or a substituted C₍₁₋₆₎. In some cases, one R^{6a} is hydrogen and the other R^{6a} is selected from C₍₁₋₆₎alkyl, or a substituted C₍₁₋₆₎ alkyl. In some cases, each R^{6a} group combine together with the nitrogen to which they are attached to form a 5 or 6-membered cyclic group. In some instances, the R^{6a} groups combined with the nitrogen to which they are attached form a 6-membered group selected from a piperidine, a piperazine, a pyridazine, a pyrazine, a triazine, a pyridine, a pyrimidine, a morpholine and a thiomorpholine. In some instances, the R^{6a} groups combined with the nitrogen to which they are attached form a 5-membered group selected from a pyrrolidine, a pyrroline, a pyrrole, an imidazolidine, a pyrazolidine, a pyrazole, an imidazole, a triazole, and a tetrazole.

While structures of formula (II-H) are drawn with substituent R⁶ at the 4′-position of the styrylpyridinium core, the substituent R⁶ (e.g., as described above) may also be present at any one or more of the other positions of the styrylpyridinium core.

In certain embodiments of a compound of formula (II-G) or (II-H), the nitrogen atom is absent, such that the core structure is a stilbene. Accordingly, the compound of (II-G) or (I-H), may have a stilbene core, which is optionally substituted (e.g., as described herein).

In certain embodiments, the fluorophore is a cinnamonitrile compound based on the following core structure:

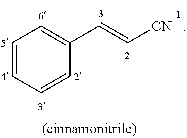

In some cases, the cinnamonitrile is further substituted at any feasible position. For example, in some cases, the core cinnamonitrile compound can be substituted at any one or more positions selected from 2, 3, 2′, 3′, 4′, 5′ and 6′ (e.g., as labeled above). In certain cases, the core cinnamonitrile compound is substituted at 1-2 positions selected from 2, 3, 2′, 3′, 4′, 5′ and 6′. In certain cases, the cinnamonitrile compound is substituted at the 2-position. In certain cases, the cinnamonitrile compound is substituted at the 2′-position. In certain cases, the cinnamonitrile compound is substituted at the 3′-position. In certain cases, the cinnamonitrile compound is substituted at the 4′-position. In certain cases, the cinnamonitrile compound is substituted at the 5′-position. In certain cases, the cinnamonitrile compound is substituted at the 6′-position.

In certain cases, the fluorophore A is described by the formula (II-I):

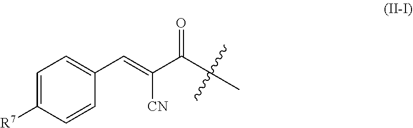

wherein:

R⁷ is selected from amino, substituted amino, alkyl, substituted alkyl, aryl, substituted aryl, acyl, carboxyl, substituted acyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-I), R⁷ is selected from an amino or a substituted amino group. In certain cases, R⁷ is a substituted amino group described by —N(R^{7a})₂, wherein each R^{7a} is independently selected from a C₍₁₋₆₎alkyl, or a substituted C₍₁₋₆₎ alkyl. In some cases, one R^{7a} is hydrogen and the other R^{7a} is selected from C₍₁₋₆₎alkyl, or a substituted C₍₁₋₆₎ alkyl. In some cases, each R^{7a} group combine together with the nitrogen to which they are attached to form a 5 or 6-membered cyclic group. In some instances, the R^{7a} groups combined with the nitrogen to which they are attached forms a 6-membered group selected from a piperidine, a piperazine, a pyridazine, a pyrazine, a triazine, a pyridine, a pyrimidine, a morpholine and a thiomorpholine. In some instances, the R^{7a} groups combined with the nitrogen to which they are attached form a 5-membered group selected from a pyrrolidine, a pyrroline, a pyrrole, an imidazolidine, a pyrazolidine, a pyrazole, an imidazole, a triazole, and a tetrazole.

While structures of formula (II-I) are drawn with substituent R⁷ at the 4′-position of the cinnamonitrile core, the substituent R⁷ (e.g., as described above) may also be present at any one or more of the 2′, 3′, 5′, or 6′ position of the cinnamonitrile core.

In certain cases, the fluorophore A is described by the formula (II-J):

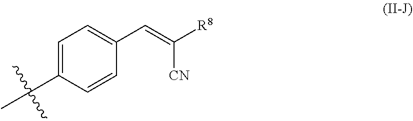

wherein:

R⁸ is selected from amino, substituted amino, alkyl, substituted alkyl, aryl, substituted aryl, acyl, substituted acyl, carboxyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-J), R⁷ is carboxyl (e.g., CO₂H). In certain cases, R⁷ is acyl or substituted acyl. In certain cases, C₍₁₋₆₎alkyl, or a substituted C₍₁₋₆₎ alkyl. In some cases, one R⁷ is amino or substituted amino.

While structures of formula (II-J) are drawn with the point of attachment to L at the 4′-position of the cinnamonitrile core, the point of attachment to L may also be present at any other position of the cinnamonitrile core.

In certain cases, the fluorophore A is a structure based on a thiazole orange core structure. In certain cases, the fluorophore A is described by the structure (II-K):

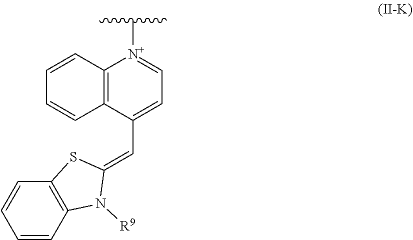

wherein:

R⁹ is selected from amino, substituted amino, alkyl, substituted alkyl, aryl, substituted aryl, acyl, substituted acyl, carboxyl, sulfonamide, substituted sulfonamide, nitro, nitrile, halogen, heteroaryl, substituted heteroaryl, heterocycle, and substituted heterocycle; and

[symbol] represents the point of attachment to L.

In certain cases of the fluorophore of formula (II-K), R⁹ is C₍₁₋₆₎alkyl, or a substituted C₍₁₋₆₎ alkyl. In some cases, one R⁹ is methyl.

In certain cases, the fluorophore A is described by the structure (II-L):

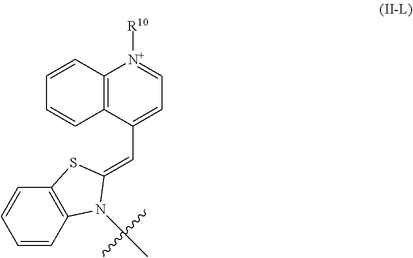

wherein:

R¹⁰ is selected from alkyl or substituted alkyl; and

[symbol] represents the point of attachment to L.

In certain cases of the fluorophore of formula (II-L), R¹⁰ is C₍₁₋₆₎alkyl, or a substituted C₍₁₋₆₎ alkyl. In some cases, R¹⁰ is methyl.

In some cases, any of the compounds of formula (II-K) or (II-L) are further substituted at any feasible position. For example, in some cases the compound of formula (II-K) or (II-L) is substituted with 1-3 additional substituents (e.g., as described above for R⁹). Further, while structures of formula (II-K) and (II-J) are drawn with particular points of attachment to L of the thiazole orange core, the point of attachment to L may also be present at any other position of the thiazole orange core.

In certain embodiments of the probe of formula (I), the fluorophore A (e.g., as described herein) is linked to the alpha nucleophile via a bond or a linker. In certain cases of a compound of formula (I), the fluorophore A is bonded directly to the alpha nucleophile, in other words, the linker group L is absent. In other cases, the fluorophore A is bonding to the alpha nucleophile via a linker L.

A variety of linking groups are known to those of skill in the art and find use in the subject compounds. Linkers of interest may include a spacer group terminated at one end with a reactive functionality capable of covalently bonding to the fluorophore A. Spacer groups of interest include aliphatic and unsaturated hydrocarbon chains, spacers containing heteroatoms such as oxygen (esters, and ethers such as polyethylene glycol) or nitrogen (amides, and polyamines), sulfur (thioesters, and dithioesters), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, esters, amides, thioesters, dithoesters, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject bifunctional molecules include heterofunctional compounds, such as azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, andsuccinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

Any convenient linker may find use in formula (I), e.g., as described herein. Suitable linkers include, but are not limited to, an alkyl moiety comprising one or more of an amine, an alkoxyl, a thiol, a PEG, and a peptide linker.

In certain embodiments of formula (I), the linker is selected from an alkyl amine, an alkyl thiol, or an alkoxy. In certain cases, the alkyl amine, alkyl thiol, or alkoxy is substituted with one or more substituents (e.g., as described herein). In certain cases, the linker is an alkyl amine, alkyl thiol or alkoxy that further includes a PEG moiety.

In certain embodiments of a probe of formula (I), L comprises a straight or branched alkyl. In certain cases, L comprises a lower alkyl group, e.g., methyl, ethyl, propyl, butyl, pentyl, or hexyl. In certain cases, L comprises a substituted alkyl group. In certain cases, L comprises a substituted lower alkyl group. In certain cases, L comprises a polyethylene glycol (PEG) or substituted PEG. In certain other cases, L is a peptide. In certain cases, L is a linear linker of 1-12 atoms in length, such as 1-10, 1-8 or 1-6 atoms in length, e.g., 1, 2, 3, 4, 5 or 6 atoms in length. The linker L can be a (C1-12)alkyl linker or a substituted (C1-12)alkyl linker, optionally substituted with a heteroatom or linking functional group, such as an ester (—CO₂—), amido (CONH), carbamate (OCONH), ether (—O—), thioether (—S—), thioester (—C(S)O—, or —C(O)S), dithioester (—CS₂—) and/or amino group (—NR— where R is H or alkyl). In certain cases, the linker L can include a keto (C═O) group. In certain cases, the keto group together with an amino, thiol or ether group in the linker chain can provide an amido, an ester or thioester group linkage. In certain cases, the linker L can include a thiocarbonyl (C═S) group. In certain cases, the thiocarbonyl group together with an amino, thiol or ether group in the linker chain can provide a thioamide, or a thioester group linkage.

In certain embodiments, the linker comprises an alkyl chain, wherein at least one of the carbon atoms of the linker backbone is optionally substituted with a sulfur, nitrogen or oxygen heteroatom. In certain cases, the linker comprises an alkyl chain wherein at least one of the carbon atoms of the linker backbone is a nitrogen atom. In certain cases, the linker comprises an alkyl chain wherein at least one of the carbon atoms of the linker backbone is an oxygen atom. In certain cases, the linker comprises an alkyl chain wherein at least one of the carbon atoms of the linker backbone is a sulfur atom.

In certain cases, the linker is an alkyl chain, wherein at least one of the carbon atoms of the linker backbone is optionally substituted with a sulfur, nitrogen or oxygen heteroatom, and the linker additionally comprises a poly(ethylene glycol unit).

In certain embodiments of a compound of formula (I), the linker is described by any one of the formulae (LI-LV):

*—NR¹¹(CR¹²₂)ₙ— (LI);

—(CR¹²₂)ₙ— (LII);

*—NR¹¹(CH₂CH₂O)ₘ(CR¹²₂)ₙ— (LIII);

*—X²(CR¹²₂)ₙ— (LIV);

*—X²(CH₂CH₂O)ₘ(CR¹²₂)ₙ— (LV);

wherein:

R¹¹ and R¹² are each independently selected from hydrogen, alkyl and substituted alkyl;

X² is O or S;

n and m are each independently an integer from 1 to 10; and

* represents the point of attachment to the fluorophore A.

In certain embodiments of the probe of formula (I), the linker is of the formula (LI). In certain cases where the linker is of formula (LI), each of R¹¹ and R¹² are hydrogen and n is less than 10, such as 9 or less, 8 or less, 7 or less, 6 or less, 4 or less, 4 or less, 3 or less, or even less. In certain cases where the linker is of formula (LI), R¹¹ is methyl, each of R¹² are hydrogen and n is less than 10, such as 9 or less, 8 or less, 7 or less, 6 or less, 4 or less, 4 or less, 3 or less, or even less. In some cases where the linker is of the formula (LI), n is 1-5, such as 1, 2, 3, 4 or 5. In certain embodiments of the probe of formula (I), the linker is of the formula (LI), where R¹¹ is selected from hydrogen or methyl, each R¹² group is hydrogen, and n is 2. In some cases, the linker is of the formula (LI), R¹¹ is hydrogen, each of R¹² is hydrogen and n is 2. In other cases, the linker is of the formula (LI), R¹¹ is methyl, each of R¹² is hydrogen and n is 2.

In certain embodiments of the probe of formula (I), the linker is of the formula (LII) and each of R¹² is hydrogen. In certain cases where the linker is of formula (LII), each of R¹² are hydrogen and n is less than 10, such as 9 or less, 8 or less, 7 or less, 6 or less, 4 or less, 4 or less, 3 or less, or even less. In some cases where the linker is of the formula (LII), n is 1-5, such as 1, 2, 3, 4 or 5.

In certain embodiments of the probe of formula (I), the linker is of the formula (LIII). In certain cases where the linker is of formula (LIII), each of R¹¹ and R¹² are hydrogen, m is 1 or 2, and n is less than 10, such as 9 or less, 8 or less, 7 or less, 6 or less, 4 or less, 4 or less, 3 or less, or even less. In certain cases where the linker is of formula (LII), R¹¹ is methyl, each of R¹² are hydrogen, m is 1 or 2 and n is less than 10, such as 9 or less, 8 or less, 7 or less, 6 or less, 4 or less, 4 or less, 3 or less, or even less. In some cases where the linker is of the formula (LIII), n is 1-5, such as 1, 2, 3, 4 or 5. In some cases where the linker is of the formula (LII), m is 1-5, such as 1, 2, 3, 4 or 5. In certain embodiments of the probe of formula (I), the linker is of the formula (LIII), where R¹¹ is selected from hydrogen or methyl, each R¹² group is hydrogen, n is 2, and m is 1 or 2. In some cases, the linker is of the formula (LI), R¹¹ is hydrogen, each of R¹² is hydrogen n is 2, and m is 1 or 2. In other cases, the linker is of the formula (LI), R¹¹ is methyl, each of R¹² is hydrogen, n is 2 and m is 1 or 2.

In certain embodiments of the probe of formula (I), the linker is of the formula (LIV). In certain cases where the linker is of formula (LIV), X² is O or S, each of R¹¹ and R¹² are hydrogen and n is less than 10, such as 9 or less, 8 or less, 7 or less, 6 or less, 4 or less, 4 or less, 3 or less, or even less. In certain cases where the linker is of formula (LIV), X² is O or S, R¹¹ is methyl, each of R¹² are hydrogen and n is less than 10, such as 9 or less, 8 or less, 7 or less, 6 or less, 4 or less, 4 or less, 3 or less, or even less. In some cases where the linker is of the formula (LIV), n is 1-5, such as 1, 2, 3, 4 or 5. In certain embodiments of the probe of formula (I), the linker is of the formula (LIV), where X² is O or S, R¹¹ is selected from hydrogen or methyl, each R¹² group is hydrogen, and n is 2. In some cases, the linker is of the formula (LIV), X² is O or S, R¹¹ is hydrogen, each of R¹² is hydrogen and n is 2. In other cases, the linker is of the formula (LIV), R¹¹ is methyl, each of R¹² is hydrogen and n is 2. In some cases where the linker is of the formula (LIV), X² is O. In some cases where the linker is of the formula (LIV), X² is S.

In certain embodiments of the probe of formula (I), the linker is of the formula (LV). In certain cases where the linker is of formula (LV), X² is O or S, each of R¹¹ and R¹² are hydrogen, m is 1 or 2, and n is less than 10, such as 9 or less, 8 or less, 7 or less, 6 or less, 4 or less, 4 or less, 3 or less, or even less. In certain cases where the linker is of formula (LV), X² is O or S, R¹¹ is methyl, each of R¹² are hydrogen, m is 1 or 2 and n is less than 10, such as 9 or less, 8 or less, 7 or less, 6 or less, 4 or less, 4 or less, 3 or less, or even less. In some cases where the linker is of the formula (LV), n is 1-5, such as 1, 2, 3, 4 or 5. In some cases where the linker is of the formula (LV), m is 1-5, such as 1, 2, 3, 4 or 5. In certain embodiments of the probe of formula (I), the linker is of the formula (LV), where X² is O or S, R¹¹ is selected from hydrogen or methyl, each R¹² group is hydrogen, m is 1 or 2, and n is 2. In some cases, the linker is of the formula (LV), X² is O or S, R¹¹ is hydrogen, each of R¹² is hydrogen, m is 1 or 2, and n is 2. In other cases, the linker is of the formula (LV), R¹¹ is methyl, each of R¹² is hydrogen, m is 1 or 2, and n is 2. In some cases where the linker is of the formula (LV), X² is O. In some cases where the linker is of the formula (LV), X² is S.

In certain cases, the linker length significantly effects the rate of conjugate formation. For example, in certain cases where the alpha nucleophile is aminooxy, the rate of oxime conjugate formation is faster for shorter linkers (e.g., when for each of (LI)-(LV), n and m are each independently 1 or 2). Accordingly, in certain instances of the linker of formula (LI), n is 1 or 2. In certain instances of the linker of formula (LII), n is 1 or 2. In certain instances of the linker of formula (LIII), m is 1 and n is 1 or 2. In certain instances of the linker of formula (LIV), n is 1 or 2. In certain cases of the linker of formula (LV), m is 1 and n is 1 or 2.

In certain embodiments, the probe of formula (I) is a compound selected from any of the following structures:

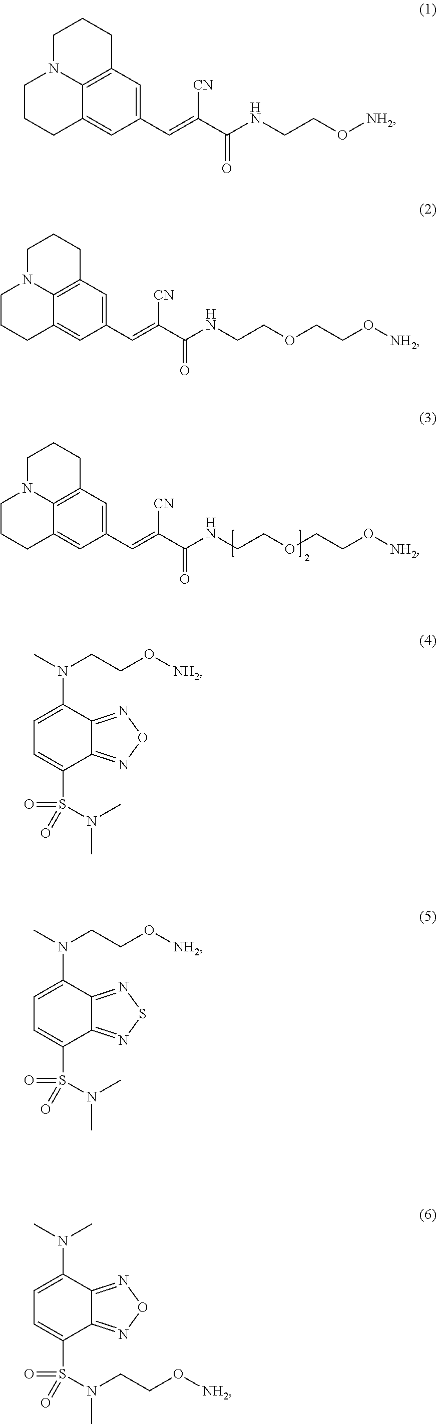

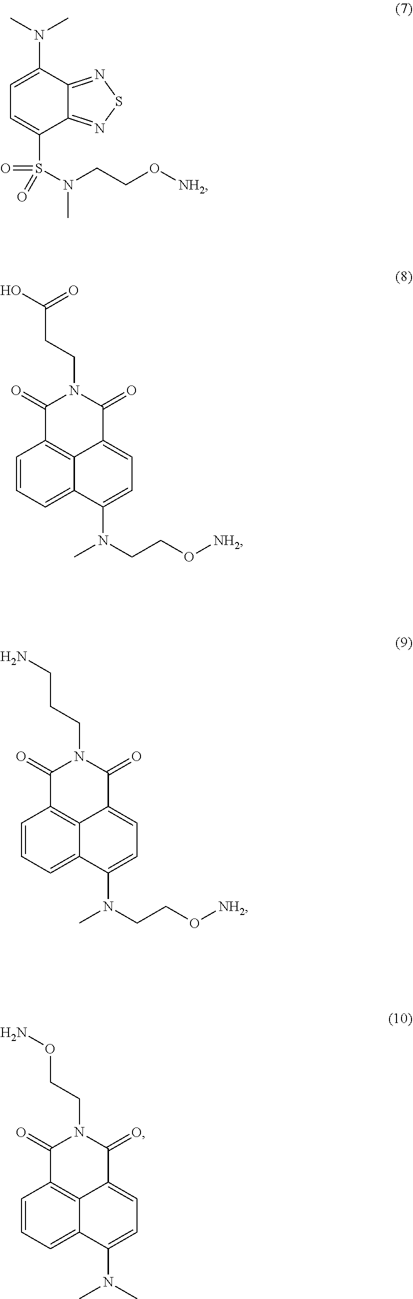 [SMILES: CN(C)c1ccc(S(=O)(=O)N(C)CCON)c2nsnc12 ; CN(CCON)c1ccc2c3c(cccc13)C(=O)N(CCC(=O)O)C2=O ; CN(CCON)c1ccc2c3c(cccc13)C(=O)N(CCCN)C2=O ; CN(C)c1ccc2c3c(cccc13)C(=O)N(CCON)C2=O]

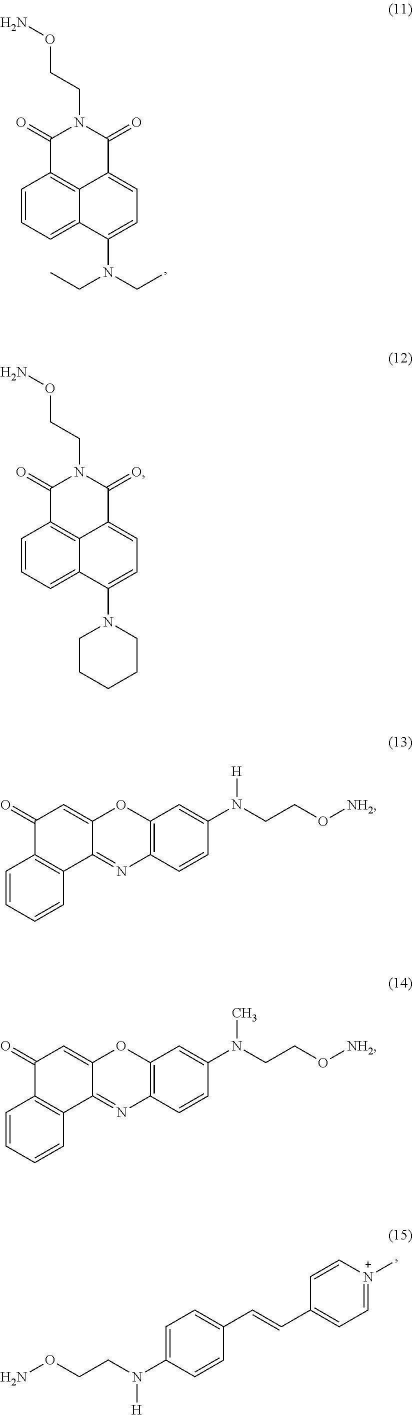

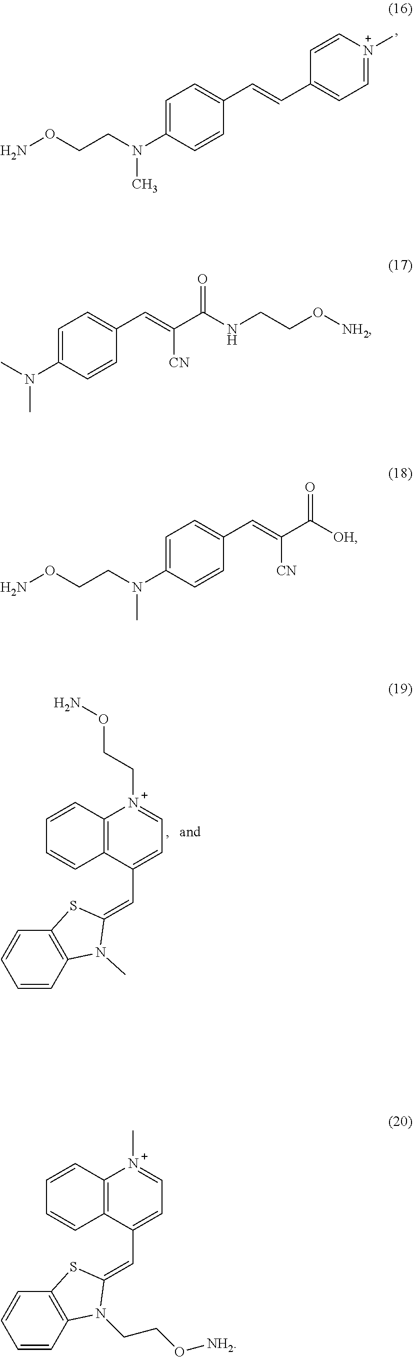

Aspects of the present disclosure include the subject compounds, salts thereof (e.g., pharmaceutically acceptable salts), and/or solvate, and hydrate forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject compounds, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-I,4-dioate, hexyne-I,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

Methods

As summarized above, this disclosure includes methods of determining glycosylase enzyme activity. Further provided herein are methods of quantifying AP sites in genomic DNA, and quantifying the amount of DNA damage. Aspects of the methods include contacting the nucleic acid with a subject probe under conditions for reaction of the alpha nucleophile of the probe with the AP sites of the nucleic acid thereby producing a conjugate; and detecting a fluorescence response generated by the conjugate to determine the presence of one or more AP sites in the nucleic acid.

The inventors surprisingly found that the subject probes react with high specificity toward the AP site of a nucleic acid (e.g., DNA) at an unprecedented rate (e.g., greater than 50 M⁻¹s⁻¹, and in some cases, about 150-300 M⁻¹s⁻¹) and affording a significant fluorescence response (e.g., in some cases ˜250-500-fold increase in fluorescence light up response relative to the unbound probe). Given the generally slow rate known for oxime formation (e.g., about 0.001-0.1 M^{-1s-1}), such rapid rate acceleration was quite surprising. The subject conjugate probe-nucleic acid conjugate assay enables facile quantification of specific glycosylase activities in vitro or in cell lysates.

Accordingly, embodiments of the methods include contacting the nucleic acid with a subject probe, wherein the probe reacts selectively with the AP sites in the nucleic acid.

In some embodiments, the reaction between the subject probe and the nucleic acid to produce the conjugate has a reaction rate of at least 50 M⁻¹s⁻¹. In some cases, the reaction rate is greater than 50 M⁻¹s⁻¹, such as 75 M⁻¹s⁻¹ or more, 100 M⁻¹s⁻¹ or more, 150 M⁻¹s⁻¹ or more, 200 M⁻¹s⁻¹ or more, 250 M⁻¹s⁻¹ or more, 250 M⁻¹s⁻¹ or more, 300 M⁻¹s⁻¹ or more, or even more. In some cases the reaction rate is from 50 to 300 M⁻¹s⁻¹, such as 75 to 300 M⁻¹s⁻¹, 100 to 300 M⁻¹s⁻¹, 150 to 300 M⁻¹s⁻¹, 200 to 300 M⁻¹s⁻¹, or 250 to 300 M⁻¹s⁻¹. In some instances, the reaction to produce the conjugate is at least 1 order of magnitude faster than a standard chemical reaction to produce the same bond at neutral pH, such as at least 2 orders of magnitude faster, at least 3 orders of magnitude faster, or at least 4 orders of magnitude faster at neutral pH. In some cases, the reaction to produce the conjugate is from 3-4 orders of magnitude faster than a standard chemical reaction to produce the same bond at neutral pH. In some instances, the reaction produces an oxime conjugate, and the reaction is at least 1 order of magnitude faster than a standard oxime bond formation at neutral pH, such as at least 2 orders of magnitude faster, at least 3 orders of magnitude faster, or at least 4 orders of magnitude faster at neutral pH. In some instances, the reaction produces a hydrazone conjugate, and the reaction is at least 1 order of magnitude faster than a standard hydrazone bond formation at neutral pH, such as at least 2 orders of magnitude faster, at least 3 orders of magnitude faster, or at least 4 orders of magnitude faster at neutral pH.

In some embodiments, the fluorescence response of the conjugate is greater than that of the probe before contacting with the nucleic acid. In some cases, the fluorescence response of the conjugate is at least 100 fold greater, such as at least 200 fold greater, at least 300 fold greater, at least 350 fold greater, at least 400 fold greater, at least 450 fold greater, at least 500 fold greater, or even greater than that of the probe before contacting with the nucleic acid. In some embodiments, the reaction produces an oxime conjugate and the fluorescence response of the oxime conjugate is greater than that of the probe before contacting with the nucleic acid. In some cases, the fluorescence response of the oxime conjugate is at least 100 fold greater, such as at least 200 fold greater, at least 300 fold greater, at least 350 fold greater, at least 400 fold greater, at least 450 fold greater, at least 500 fold greater, or even greater than that of the probe before contacting with the nucleic acid. In some embodiments, the reaction produces a hydrazone conjugate and the fluorescence response of the hydrazone conjugate is greater than that of the probe before contacting with the nucleic acid. In some cases, the fluorescence response of the hydrazone conjugate is at least 100 fold greater, such as at least 200 fold greater, at least 300 fold greater, at least 350 fold greater, at least 400 fold greater, at least 450 fold greater, at least 500 fold greater, or even greater than that of the probe before contacting with the nucleic acid.

### Uses of Probes

By simultaneous labeling of the AP site and activation of probe fluorescence, the subject probes can find use in reporting real-time base excision activity. Furthermore, by employing the probe with substrates containing a variety of DNA lesions, the subject probes can be used to measure any potential glycosylase or substrate of interest.

Because of its unusual rate acceleration and light up mechanism, the UBER probe design offers very low background and low off-target light up signals, even in the presence of high concentrations of common small-molecule carbonyl compounds and the complex matrix of cellular lysates. The ability to monitor the dynamic response of glycosylase activity in cell lysates allows efficient glycosylase activity profiling which previously would have only been achieved through lower sensitivity fluorescence methods, which could require days to develop an observable signal.

An advantage of the subject “UBER” probes is their ability to provide for highly selective, robust and rapid fluorescence signals in response to abasic sites generated during DNA base excision repair. In some instances, the alpha nucleophile of the subject probe is an aminooxy moiety, and the probe reacts with the abasic sites to produce an oxime conjugate. The unprecedentedly fast oxime formation reaction allows reactions to proceed in a time frame suitable for high throughput screening assays. The UBER probes combine sensitivity, speed and generalizability in a continuous assay that can be adapted for use with virtually any human glycosylase

The UBER probes have significant advantages over previously reported methods for assaying DNA glycosylase activity. Traditional biochemical methods such as gel-based assays or radiation release assays are discontinuous and therefore time and labor intensive. In contrast, the UBER probes allow for a continuous fluorescence assay, where an entire screen can be completed on a single microplate in a matter of hours. Although molecular beacon probes of base excision are continuous, they rely on strand cleavage and therefore cannot be used with monofunctional glycosylases, which represent the majority of human glycosylases, without further downstream processing.

Since the probe and substrate are independent from one another, the UBER probe assay can be implemented without synthesizing lesion-containing oligonucleotide substrates. This is particularly advantageous for cases where the DNA lesion of interest is costly or challenging to incorporate into a synthetic oligonucleotide using conventional phosphoramidite synthesis. By utilizing a separate reporter molecule, the UBER probe can be paired in a coupled assay with any DNA substrate without further modification. This can allow sensing of candidate, uncharacterized DNA glycosylase activity. Enzyme substrates are commercially available or can be generated in situ from biologically derived DNA. By utilizing native substrates rather than modified reporter oligonucleotides, the UBER probe produces no interference with native enzyme activity. Unlike previous probes that rely on secondary DNA cleavage (lyase) activity to generate signal, the UBER probe generates signal in direct proportion to base excision and requires no secondary enzyme activity. This large fold change allows sensitive detection of DNA glycosylase activity in cell lysates in a relatively short amount of time.

Measurement of Glycosylic Enzyme Activity

UBER probes are useful in quantitating the presence of AP sites in nucleic acids, including without limitation DNA. This quantitation of AP sites can be correlated to provide a measure of glycosylase activity. In some embodiments, the nucleic acid is DNA that has been contacted with a glycosylase enzyme to generate DNA with AP sites. Quantitation of AP sites following glycosylase activity provides a measure of the activity of the glycosylase enzyme. In certain aspects, the nucleic acid is a purified genomic DNA and the method further comprises comparing the fluorescence response of the conjugate to a standard to quantify the prevalence of AP sites in the purified genomic DNA. In certain cases, where the nucleic acid is a purified genomic DNA, the method further includes a pretreating step where the DNA is contacted with a corresponding DNA repair enzyme before contacting the DNA with the probe. The number of AP sites in the pre-treated sample is then compared to the number of AP sites in an untreated DNA sample to quantify the amount of DNA damage. Also provided herein are kits including a subject probe and a DNA repair enzyme.

An UBER probe can be contacted with a candidate nucleic acid at a concentration that allows reporter activity. The concentration may be empirically determined, or a set of limiting dilution assays. The concentration of probe in the reaction may be, for example, from 0.01 μM; 0.05 μM; 0.1 μM; 0.5 μM; 1 μM; 5 μM; 10 μM; 15 μM; 25 μM; 50 μM; or more. A DNA substrate, e.g. an oligonucleotide of from 10, 20, 30, 40, 50 60, 70, 80, 90 100 bases or more in length may be added to the assay to test for the presence of candidate glycosylase activity, where the substrate is added at a concentration of from 0.01 M; 0.05 M; 0.1 M; 0.5 M; 1 M; 5 M; 10 M; 15 M; 25 M; 50 M; or more. Hairpin or otherwise double stranded substrates are of interest. Alternatively, native nucleic acids present in a sample can be assayed.

It is shown herein that the UBER probe can efficiently report on base excision activity from a variety of glycosylases in vitro, and can be used to profile endogenous glycosylase activity in cell lysates.

UBER probes can be used to measure dynamic changes in glycosylase expression level in response to environmental stimuli or disease states using a simple mix-and-measure format. Such assays can be used to generate enzyme rate velocities. Even enzymes expressed at low levels, or with a turnover rate, for example from about 1-10 s⁻¹, are readily assayed with an UBER probe.

Candidate glycosylase enzymes or agents that are inhibitors of glycosylase enzymes can be screened. Candidate are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Test compounds may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, seawater, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of wells, where the sample either comprises a candidate nucleic acid, or where a nucleic acid substrate is added. The change in signal readout in response to the agent is measured, desirably normalized, and the resulting results may then be evaluated by comparison to controls and other reference data.

The agents are conveniently combined in solution, or readily soluble form. The agents may be added in a flow-through system, as a stream, intermittent or continuous, or alternatively, adding a bolus of the compound, singly or incrementally, to an otherwise static solution. In a flow-through system, two fluids are used, where one may be a physiologically neutral solution, and the other is the same solution with the test compound added. The first fluid is passed over the cells, followed by the second. In a single solution method, a bolus of the test compound is added to the sample volume.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the fluorescence. Various methods can be utilized for quantifying fluorescence, as known in the art.

Other embodiments described herein relate to the use of the UBER probes in direct imaging and quantifying the generation of AP sites in DNA and assessing DNA damage and repair. Direct imaging and quantitative assessment of AP sites in DNA can be used for efficacy evaluation of DNA-targeted chemotherapies and/or anticancer agents that produce AP sites and invoke base excision repair (BER). Understanding the dynamic of AP site formation and repair can allow clinicians and researchers to determine optimal dose strategies of single and combination chemotherapeutic treatment schedules. Furthermore, direct imaging of AP sites in DNA can be used to determine the optimal dose schedule to potentiate drug administration based on persistence of AP sites. For instance, if one agent induces AP sites, and another blocks BER repair, while a third induces Topo II, understanding the relationship between these events can impact therapeutic efficacy. In addition, direct imaging of AP sites can facilitate screening of new agents that are designed to either induce AP sites in tumor or cancer cells or block AP sites from DNA repair.

By way of example, a sample of DNA can be isolated from a biological sample, such as a biological sample obtained from a subject under examination and/or a subject treated with a DNA damaging agent, such as an anti-neoplastic agent and/or anti-mitotic agent. The biological sample obtained from the subject can include blood, tissue, as well individual cells. In one example, the sample of DNA can be isolated from peripheral blood mononuclear cells obtained from a subject. The DNA sample can be isolated from the biological sample using conventional DNA isolation and purification methods.

Following isolation of the DNA from the biological sample, the isolated DNA sample can be contacted with the UBER probes described herein that bind to AP sites of the DNA. The fluorescent probes can be provided in a buffer to provide an AP detection reagent. Following contact of the DNA sample with the UBER probe, the fluorescence can be detected and quantitated. The fluorescent probe labeled AP sites can be quantitatively detected fluorometrically or through other types of electromagnetic spectroscopy, which analyze fluorescence from the sample. Devices that measure fluorescence are commonly referred to as fluorometers, fluorimeters, or fluorescence spectrophotometers.

The measured fluorescence can be compared with the fluorescence of standard control specimens of known AP-DNA concentrations to quantitate or determine the number of AP sites in the DNA sample. Blank AP-DNA background readings from the control DNA can also be used to quantitatively determine the number of AP sites of the DNA sample. In some embodiments, the concentration of AP sites in the DNA sample can be quantitatively determined by plotting the fluorescence intensity versus the concentration of AP sites of the DNA sample. The concentration AP sites of the DNA sample can then be correlated with the concentration of plotted AP sites of the control specimen to determine the amount of AP-DNA in the isolated DNA from the biological sample.

To produce control DNA of known AP-DNA concentrations against which the sample of DNA can be compared, a DNA sample, for example double stranded calf thymus DNA, can be obtained and specific numbers of AP sites can be selectively produced, as known in the art. Typically a heat/acid depurination buffer treatment can be used to produce useful control samples. Multiple working solution AP-DNA controls of varying concentrations can be produced and utilized in the methods provided. In one embodiment, controls and samples can be assayed by treating the sample of DNA and control DNA specimens in parallel so that the sample and control DNA specimen(s) are each subjected to the same or similar environmental and process conditions so as to remove any such variables from the respective samples when interpreting the results of their comparisons.

In some embodiments, the fluorescent probe can be used to measure the efficacy of an anticancer agent in generating AP sites in cancers cells of a subject to which the anticancer agent is administered. Measuring the ability of the anticancer agent to generate AP sites in the cancer cells can be used to determine whether a specific anticancer is effective in treating a subject or a specific cancer. If an anticancer agent administered to a subject is found to not generate AP sites, a therapy using an anticancer agent can be halted and another or different anticancer agent can be selected and be administered to the subject. Additionally, the amount or quantity of AP sites generated by an anticancer agent in a subject to which the anticancer agent is administered can be measure and quantified using the fluorescent probe to determine the efficacy of the therapy. For example, the fluorescent probe can be used to measure quantity of AP sites generated by an anticancer agent. The greater the number or amount of AP sites generated in cancer cells of the subject measured using the fluorescent probe the more effective the anticancer agent can be at treating the cancer in the subject.

Non-limiting examples of anticancer agents that induce the formation of AP sites in cancer cells of a subject are intercalating agents, such as bleomycin, adriamycin, quinacrine, echinomycin (a quinoxaline antibiotic), and anthrapyrazoles. Radiation, such as gamma radiation, UVA, and UVB, can also be used to generate AP sites according to the methods of the invention. Ultraviolet light is absorbed in DNA with the formation of UV-specific di-pyrimidine photoproducts. Exposure to gamma irradiation, UVA, and UVB can induce damaged pyrimidine photodimers. Anticancer agents that induce the formation of AP sites can also include alkylating agents, such as temozolomide (TMZ), 1,3-bis(2-chloroethyl)-I-nitrosourea (BCNU), MeOSO₂(CH₂)₂-lexitropsin (Me-Lex), cis-diamminedichlo-roplatinum II (cisplat; cis-DDP), mitomycin bioreductive alkylating agents, quinones, streptozotocin, cyclophosphamide, nitrogen mustard family members such as chloroambucil, pentostatin (and related purine analogs), fludarabine, bendamustine hydrochloride, chloroethylating nitrosoureas (e.g., lomustine, fotemustine, cystemustine), dacarbazine (DTIC), and procarbazine. In certain embodiments, the alkylating agent is a nitrosourea, such as a mustine, carmustine, fotemustine, lomustine, nimustine, ranimustine, or semustine. Other agents include radiosensitizers, such as 5-iodo-2′-deoxyuridine (IUdR), 5-fluorouracil (5-FU), 6-thioguanine, hypoxanthine, uracil, fludarabine, ecteinascidin-743, and camptothecin and analogs thereof.

### Kits

Aspects of the invention further include kits for use of the subject probes in practicing the subject methods. The compounds of the invention can be included as reagents in kits for use in, for example, the methodologies described above.

In one embodiment there is provided a kit including, a subject probe (e.g., as described herein), and a DNA repair enzyme.

A kit can include a probe (e.g., as described herein); and one or more components selected from the group consisting of an additional active agent, a buffer, a solvent, a standard and instructions for use.

The one or more components of the kit may be provided in separate containers (e.g., separate tubes, bottles, or wells in a multi-well strip or plate).

The probes and other components of the kits may be provided in a liquid composition, such as any suitable buffer. Alternatively, the probes and components of the kits may be provided in a dry composition (e.g., may be lyophilized), and the kit may optionally include one or more buffers for reconstituting the dry compound. In certain aspects, the kit may include aliquots of the probe or other components provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

Other embodiments described herein relate to a kit for assaying AP site of a DNA sample. The kit can include a control DNA specimen having a known concentration of AP-sites and an AP detection reagent that include the fluorescent probe. The kit can also include instructions to explain how one may fluorometrically compare a given sample of DNA and control DNA. The instructions can further include directions on contacting the sample DNA and a set of control DNA specimens each having a known number of AP sites with the AP detection reagent. The kit may also include further instructions on performing fluorometric analysis to correlate the amount of AP-sites in a sample of DNA relative to the control DNA specimens.

### Utility

The compounds and methods of the invention, e.g., as described herein, find use in a variety of applications. Applications of interest include, but are not limited to: research applications and therapeutic monitoring applications. Methods of the invention find use in a variety of different applications including any convenient application where measurement of glycosylic enzyme activity, DNA repair, or quantification of DNA damage is desired.

The subject compounds and methods find use in a variety of research applications. For example, the subject compounds and methods may be used in high throughput screening of potential glycosylase inhibitors.

The subject compounds and methods find use in a variety of applications such as measuring and monitoring DNA repair, quantification of AP sites in genomic DNA and quantification of genomic DNA damage. These types of assays find use in biomedical research as well as basic life sciences research, such as cancer research. The accumulation of damage in genomic DNA is known to contribute significantly to cancer progression, and the mechanisms by which this occurs are of considerable interest to researchers.

As such, the subject probes find use in applications where assessing the extent of DNA damage and quantifying the activity of certain DNA repair enzymes is desired (e.g., as described herein).

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celcius, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Background

The study of the mechanisms of DNA damage and repair is important to understanding the origins of cancer. DNA glycosylases are a class of DNA repair enzyme responsible for initiating base excision repair (BER). Enzymes of this broad class recognize damaged or mispaired DNA bases and hydrolyze the N-glyosidic bond between the targeted base and the sugar (FIG. 1). The resulting hemiacetal abasic (AP) site created by base excision is then cleaved and ultimately filled in by downstream repair enzymes using the complementary strand to preserve the original genetic information.

The AP site generated in DNA by base excision potentially constitutes a similarly constrained, water-excluded target site, which it was hypothesized could be exploited to constrain a TICT or rotor-based probe (FIG. 1). The hemiacetal AP site is in equilibrium with its aldehyde form, which provides a convenient handle that can be targeted with reactive alpha nucleophiles.

In an effort to meet these aspects, it was sought to develop a universal base excision reporter (UBER) probe design that could allow measurements of any glycosylase using a single small-molecule reporter.

Herein is described the design and synthesis of aminooxy functionalized, fluorescence probes (also referred to herein as fluorescence light-up probes) that undergo ultrafast oxime formation to measure DNA base excision in real-time. The molecular rotor-based design reacts with high specificity toward the AP site of DNA at an unprecedented rate (˜150-300 M⁻¹s⁻¹) and affording a 250-500-fold fluorescence light up response. The coupled UBER probe assay allows facile quantification of specific glycosylase activities in vitro or in cell lysates. To test the ease of use and utility of the UBER probe, activities were measured of UNG and OGG1 in cell lysates representing DNA glycosylases with both high and low turnover numbers respectively.

### Example 1: Probe Design and Synthesis

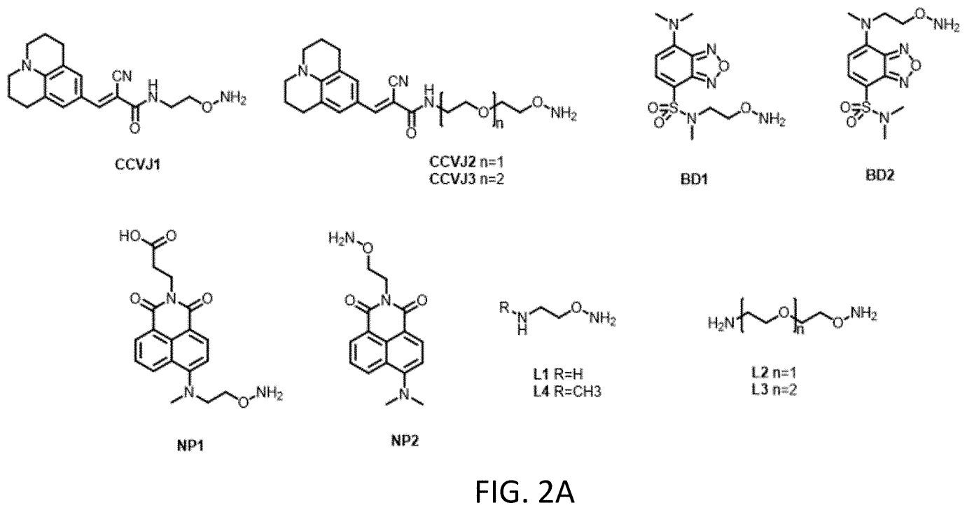

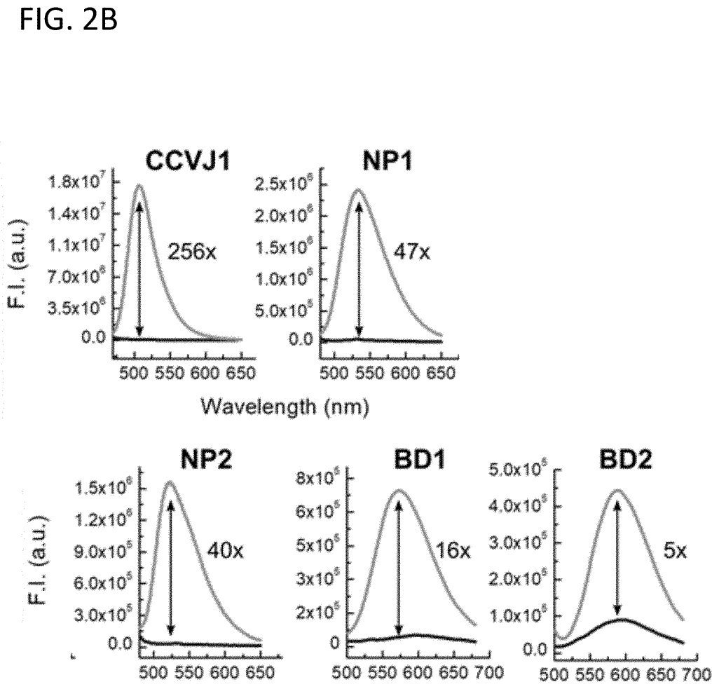

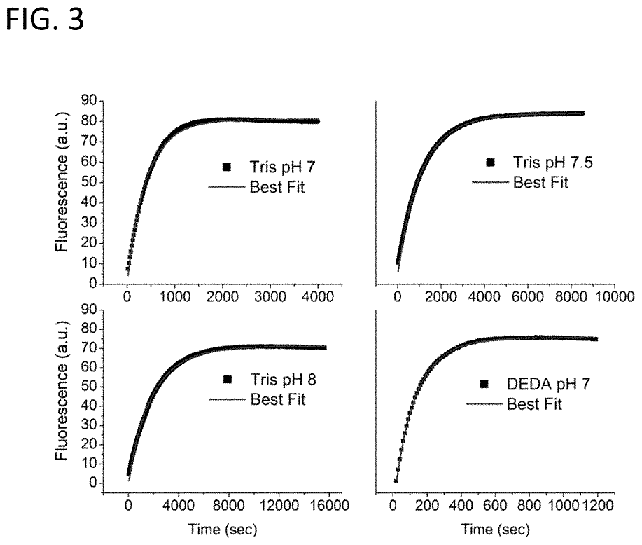

To begin the study, aldehyde-reactive linker L1 and the N-methylated L4 were synthesized using previously reported chemistry (FIG. 2A). The aminooxy alpha nucleophile and oximes were selected. Linkers L1 and L4 were attached to three fluorophores that have been previously reported as TICT probes or molecular rotors. Specifically, 1,8-naphthalimide fluorophore, the molecular rotor 9-(2-carboxy-2-cyanovinyl)julolidine (CCVJ) and the environmentally sensitive benzoxadiazole dye, yielding probes NP1, CCVJ1, and BD1 respectively (see, e.g., FIG. 2A).

CCVJ Based Probes

Boc protected aminooxy linkers L1-L3 were prepared as HCl salts by the method of Carrasco and coworkers and 9-(2-Carboxy-2-cyanovinyl)julolidine (CCVJ) was prepared as described by Rumble and coworkers.

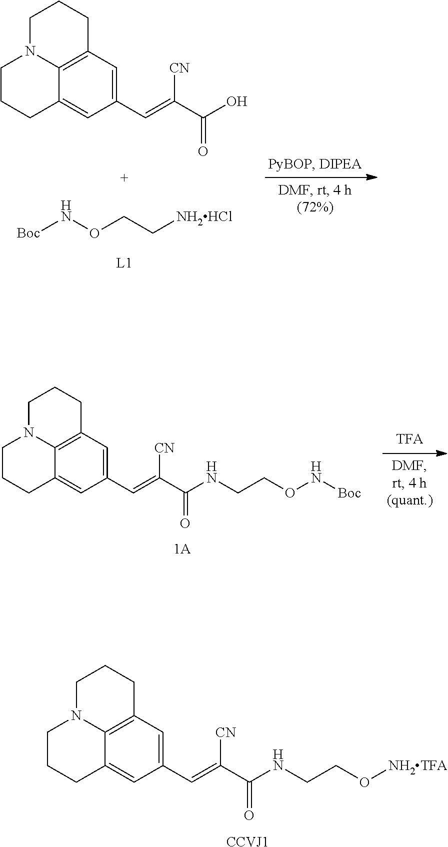

Compound 1A. CCVJ (107 mg, 0.4 mmol), L1 (85 mg, 0.4 mmol), PyBOP (229 mg, 0.44 mmol) and DIPEA (0.35 mL, 2 mmol) were dissolved in dry DMF (1 mL) and stirred for 1 hr. The reaction was then taken into ethyl acetate (15 mL), washed with 1M HCl and saturated brine, dried over anhydrous magnesium sulfate and concentrated in vacuo. The resulting crude was purified by FCC (EtOAc:Hexanes 2:3) yielding 123 mg (72%) of an orange foam.

Compound 1 (CCVJ1)

Compound 1A (20.2 mg, 47.3 umol) was dissolved in 800 μL of a 1:1 solution of dry DCM and TFA. After 4 hours, the volatiles were evaporated under a continuous stream of Argon. Remaining TFA was co-evaporated by the addition of 1 drop Toluene yielding 20.0 mg (quant.) of a red-orange residue.

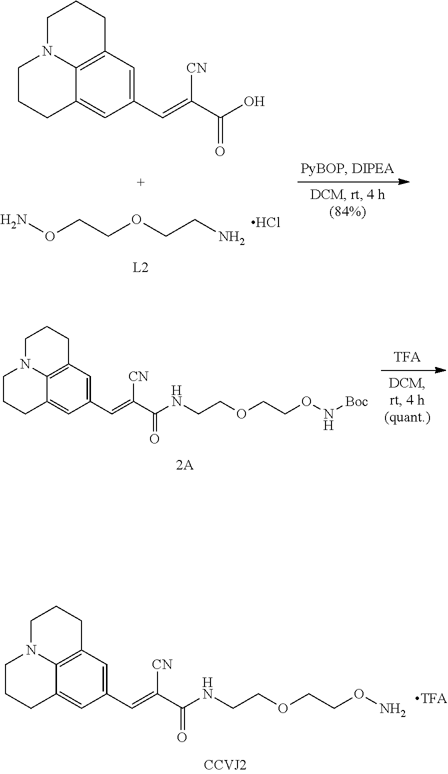 [SMILES: N#C/C(=C\c1cc2c3c(c1)CCCN3CCC2)C(=O)O ; NCCOCCON ; Cl ; CC(C)(C)OC(=O)NOCCOCCNC(=O)/C(C#N)=C/c1cc2c3c(c1)CCCN3CCC2 ; N#C/C(=C\c1cc2c3c(c1)CCCN3CCC2)C(=O)NCCOCCON ; "·TFA"]

Compound 2A

CCVJ (107 mg, 0.4 mmol), L2 (103 mg, 0.4 mmol), PyBOP (229 mg, 0.44 mmol) and DIPEA (0.35 mL, 2 mmol) were dissolved in dry DMF (1 mL) and stirred for 1 h. The reaction was then taken into ethyl acetate (15 mL), washed with 1M HCl and saturated brine, dried over anhydrous magnesium sulfate and concentrated in vacuo. The resulting crude was purified by FCC (EtOAc:hexanes 1:1) resulting in 158 mg (84%) of an orange foam.

Compound 2 (CCVJ2). Compound 2 (20.5 mg, 43.6 umol) was dissolved in 800 μL of a 1:1 solution of dry DCM and TFA. After 4 hours, the volatiles were blown off under a continuous stream of Argon. Remaining TFA was co-evaporated by the addition of 1 drop toluene yielding 20.4 mg (quant.) of a red-orange residue.

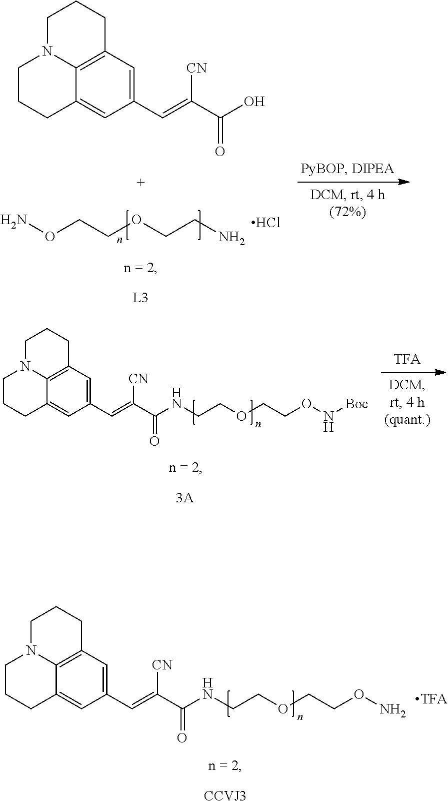

Compound 3A. CCVJ (107 mg, 0.4 mmol), L3 (120 mg, 0.4 mmol), PyBOP (229 mg, 0.44 mmol) and DIPEA (0.35 mL, 2 mmol) were dissolved in dry DMF (1 mL) and stirred under an atmosphere of Argon for 1 hr. The reaction was then taken into ethyl acetate (15 mL), washed with 1M HCl and saturated brine, dried over anhydrous magnesium sulfate and concentrated in vacuo. The resulting crude was purified by FCC (EtOAc:hexanes 3:2) resulting in 148 mg (72%) of a red-orange foam.

Compound 3 (CCVJ3). Compound 3 (17.0 mg, 33.0 umol) was dissolved in 800 μL of a 1:1 solution of dry DCM and TFA. After 4 hours, the volatiles were evaporated under a continuous stream of Argon. Remaining TFA was co-evaporated by the addition of 1 drop toluene yielding 17.0 mg (quant.) of a red-orange residue.

Naphthalimide Based Probes

Boc protected aminooxy linkers L1 and L4 were prepared as HCl salts by the method of Carrasco and coworkers and compound 4A was prepared as described by Lee and coworkers.

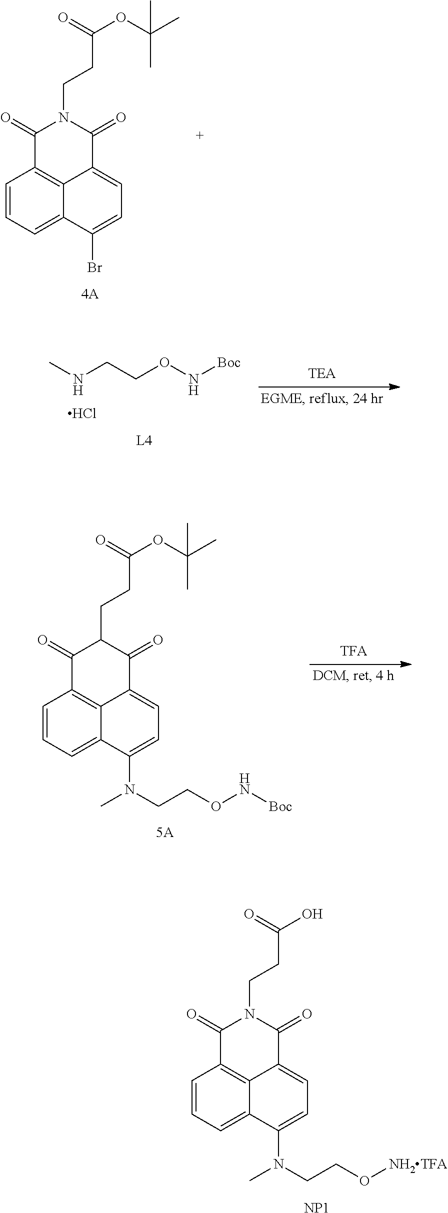

Compound 5A. Compound 4A (202 mg, 0.5 mmol) and L4 (227 mg, 1.0 mmol) was dissolved in 2-methoxyethanol (1 mL) and trimethylamine (0.27 mL, 2 mmol) was added. The solution was stirred at 120° C. for 24 hours. The reaction was then cooled and concentrated in vacuo. The resulting crude residue was purified by FCC (1:1 EtOAc:Hexanes) yielding 48.5 mg (19%) of an orange foam.

Compound 8 (NP1). Compound 5A (19.7 mg, 38.3 umol) was dissolved in 800 μL of a 1:1 solution of dry DCM and TFA. After 4 hours, the volatiles were evaporated under a continuous stream of argon. Remaining TFA was co-evaporated by the addition of 1 drop Toluene yielding 17.5 mg (quant.) of an orange residue.

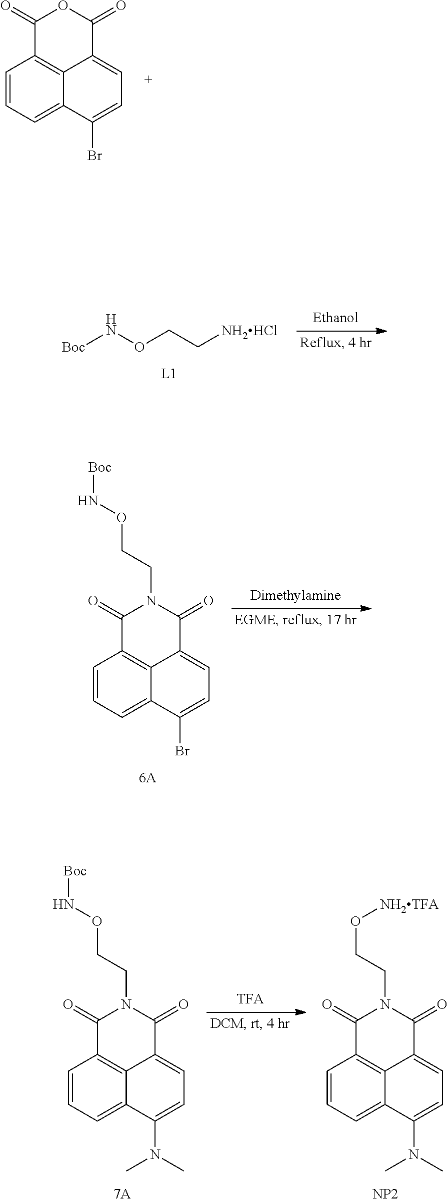

Compound 6A. 4-bromo-1,8-naphthalic anhydride (277 mg, 1 mmol) and L1 (234 mg, 1.1 mmol) were suspended in ethanol (3 mL) and heated to reflux while stirring for 4 h. The solution was cooled and the product was isolated by vacuum filtration yielding 213 mg of a grey solid (61%) that was used without further purification.

Compound 7A. Compound 6 (87 mg, 0.2 mmol) was dissolved in 2-methoxyethanol (2 mL) along with dimethylamine (0.25 mL, 2 mmol). The solution was stirred at 120° C. for 2 hours. The solution was cooled and the reaction concentrated in vacuo. The resulting crude residue was purified by FCC (4:1 EtOAc:Hexanes) yielding 32.6 mg (41%) of an orange foam.

Compound 10 (NP2). Compound 5A (32.6 mg, 81.5 umol) was dissolved in 800 μL of a 1:1 solution of dry DCM and TFA. After 4 hours, the volatiles were evaporated under a continuous stream of argon. Remaining TFA was co-evaporated by the addition of 1 drop toluene yielding 27.3 mg (quant.) of an orange residue.

Benzoxadiazole Based Probes

Boc protected aminooxy linkers L1 and L4 were prepared as HCl salts by the method of Carrasco and coworkers and 7-chloro-N,N-dimethyl-2,1,3-benzoxadiazole-4-sulfonamide was prepared as described by Pagano and coworkers.

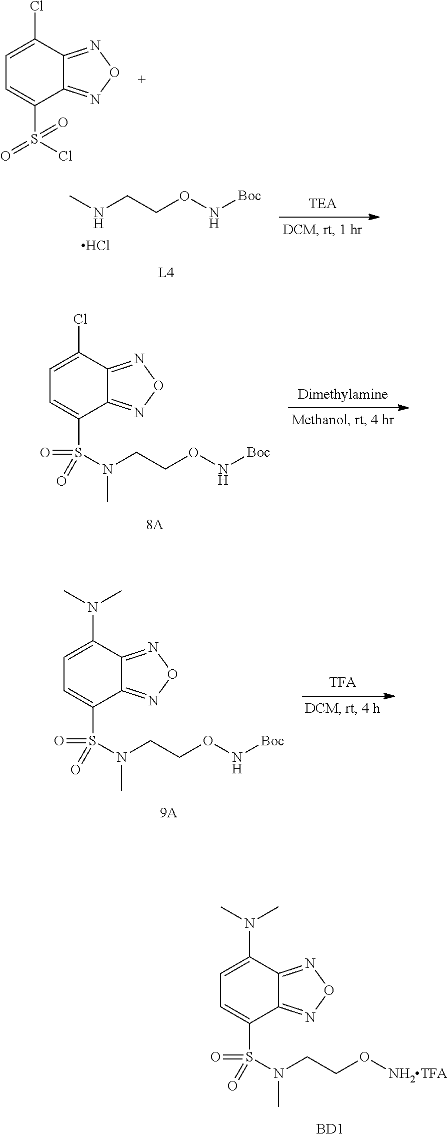

Compound 8A. 7-Chloro-2,1,3-benzoxadiazole-4-sulfonyl chloride (253 mg, 1 mmol) and L4 (249 mg, 1.1 mmol) were dissolved in dry DCM (12 mL). Trimethylamine (0.34 mL, 2.5 mmol) was added dropwise and the solution was stirred for 1 hr. The reaction was then diluted with DCM (50 mL) and washed with 1M HCl and concentrated brine. The organic fraction was dried over anhydrous magnesium sulfate and concentrated in vacuo to yield 342 mg (84%) of a yellow-green oil.

Compound 9A. Compound 8A (342 mg, 0.85 mmol) was dissolved in methanol (3 mL) and dimethylamine was added dropwise (0.67 mL, 8.5 mmol). The solution was stirred at room temperature for 4 hrs. The reaction was then concentrated in vacuo. The resulting crude residue was re-dissolved in EtOAc (25 mL) and washed with 1M HCl and concentrated brine. The organic fraction was dried over anhydrous magnesium sulfate and concentrated in vacuo to yield 315 mg (89%) of an orange foam.

Compound 6 (BD1). Compound 9A (21.9 mg, 52.7 umol) was dissolved in 800 μL of a 1:1 solution of dry DCM and TFA. After 4 hours, the volatiles were evaporated under a continuous stream of Argon. Remaining TFA was co-evaporated by the addition of 1 drop toluene yielding 21.8 mg (quant.) of an orange residue.

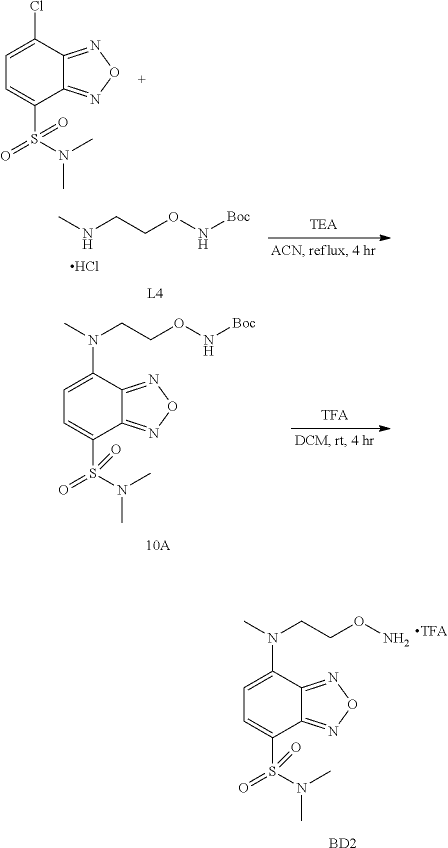

Compound 10A. 7-chloro-N,N-dimethyl-2,1,3-benzoxadiazole-4-sulfonamide (105 mg, 0.4 mmol) and L4 (181 mg, 0.8 mmol) was dissolved in dry acetonitrile (5 mL) with trimethylamine (0.28 mL, 2 mmol). The solution was heated to reflux while stirring for 4 h. The reaction was then concentrated in vacuo, taken up into EtOAc (25 mL) and washed with 1M HCl and concentrated brine. The organic fraction was concentrated in vacuo yielding 154 mg (92%) of a yellow-green oil.

Compound 4 (BD2). Compound 10A (22.8 mg, 54.9 umol) was dissolved in 800 μL of a 1:1 solution of dry DCM and TFA. After 4 hours, the volatiles were evaporated under a continuous stream of argon. Remaining TFA was co-evaporated by the addition of 1 drop toluene yielding 22.7 mg (quant.) of an orange residue.

### Example 2: Fluorescence Responses

To assess the potential fluorescence response of each probe, a 17mer DNA hairpin containing a single AP site was prepared and reacted it with a 10-fold excess of probe overnight. The subsequent DNA-probe conjugates were precipitated to remove excess fluorophore and full DNA labelling was confirmed by MALDI-TOF mass spectrometry (Table 1). The reacted DNAs were re-suspended in buffer to a concentration of 2 μM and the emission spectra compared with the spectra of the free probes in solution. It was found that in comparison to the free dye, all three probes demonstrated increased fluorescence when covalently attached to the DNA AP site (FIG. 2B). In particular it was observed that exemplary compound CCVJ1 (also referred to herein as Compound 1) yielded a dramatic 256-fold increase in fluorescence when bound to the AP site. In addition to its large fluorescence response, CCVJ1 is accessed readily through a facile 4-step synthesis and has good spectral overlap with fluorescein. This makes the probe accessible to a wide range of researchers and well suited to the majority of fluorescence-based instruments.

TABLE 1
MALDI-TOF Data.

| Species | Calc'd mass | Observed mass |
|---|---|---|
| Oligo 15 | 5206.41 | 5210.04 |
| Oligo 15 + UDG (AP Site) | 5112.34 | 5114.93 |
| CCVJ1 Conjugate | 5420.72 | 5423.93 |
| CCVJ2 Conjugate | 5464.77 | 5465.05 |
| CCVJ3 Conjugate | 5508.83 | 5511.49 |
| NP1 Conjugate | 5451.69 | 5451.56 |
| NP2 Conjugate | 5393.65 | 5396.57 |
| BD1 Conjugate | 5409.67 | 5412.31 |
| BD2 Conjugate | 5409.67 | 5409.88 |
| Oligo 21 | 5236.44 | 5237.9 |
| Oligo 22 | 5221.42 | 5220.6 |

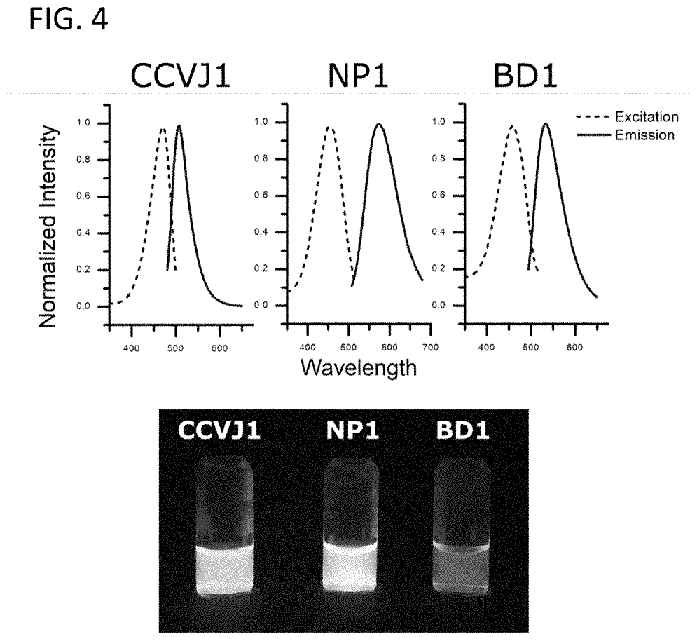

Since the probes based on 1,8-naphthalimide and benzoxadiazole had multiple potential linker attachment sites, also synthesized were NP2 and BD2 with altered attachment points. While NP2 demonstrated similar photophysical properties to NP1, the synthesis and purification of NP2 was simpler, making it the preferred 1,8-naphthalimide based probe. Due to the close overlap in excitation spectra of all three probe designs, they can easily be excited at a common wavelength (440-480 nm) and produce a multicolor output for multiplexing applications if desired (FIG. 4).

### Example 3: Oxime Formation Kinetics

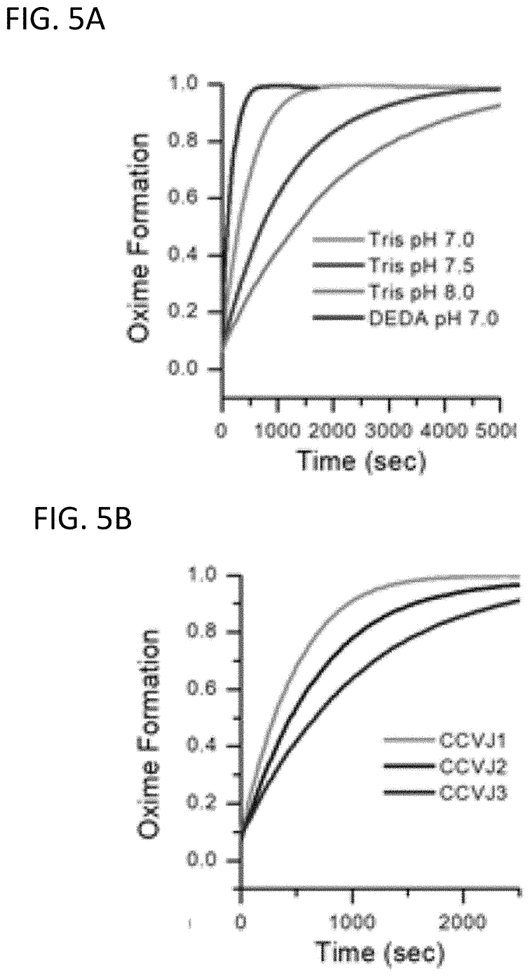

Next the reaction rates with CCVJ1 were evaluated. To measure oxime formation kinetics, 5 μM CCVJ1 was reacted with 20 μM AP DNA, and the resulting fluorescence time course was used to determine second-order rate constants by nonlinear regression analysis. Tris buffer was selected since it is commonly used in glycosylase assays with a salt concentration of 100 mM NaCl. It was found that probe CCVJ1 demonstrated remarkably rapid oxime formation kinetics (FIG. 5A). At pH 7.0, CCVJ1 reacted with an apparent second-order rate constant of 147 M⁻¹s⁻¹ (Table 2), 10³-10⁴ times faster than typical oxime formation in neutral aqueous buffer. These results are of interest because rapid oxime formation led to an efficient coupled assay (see below). It was found that second-order rate constants decreased with increasing pH.

TABLE 2
Apparent second-order rate constants of oxime
formation between CCVJ1 and AP-DNA

| Buffer | k₂ (M⁻¹s⁻¹) |
|---|---|
| Tris pH 7 | 147 ± 8 |
| Tris pH 7.5 | 51 ± 2 |
| Tris pH 8 | 29 ± 1 |
| DEDA pH 7 | 440 ± 12 |

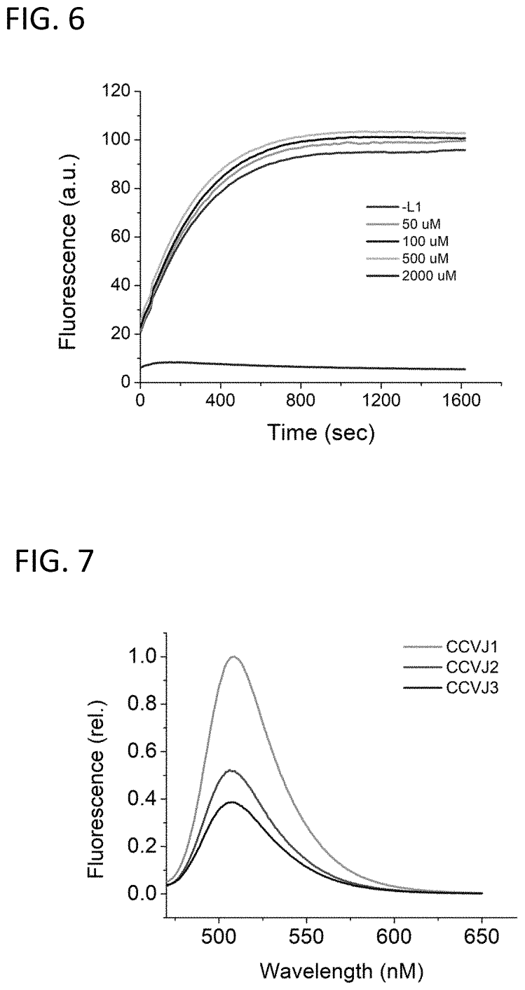

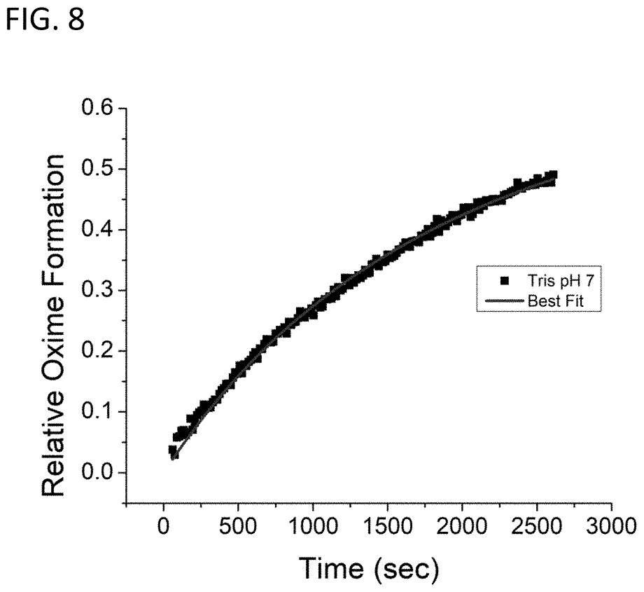

Additional experiments were performed to study the origins of this unusually rapid reaction. A study by Kojima et al. demonstrated that a naphthalene moiety in close proximity to an aminooxy group accelerated the rate of oxime formation with double stranded AP DNA ˜3-fold over a probe with a longer linear linker. The authors suggested that the planar naphthalene accelerates oxime formation by pre-binding the DNA through stacking interactions. The maximum rate observed in their study, however, was 0.005 M⁻¹s⁻¹ at pH 8, over 5000 times slower than the k₂ that was observed with CCVJ1 at pH 8 (Table 2). To test the effect that proximity between the alkoxyamine and planar portion of the probe had on oxime kinetics in our probes, probes were synthesized having variable-length linkers (CCVJ2 and CCVJ3) from aldehyde reactive linkers L2 and L3 respectively (FIG. 2A). It was found that homologating the linker length of CCVJ1 by one or two ethylene glycol units slowed the reaction rate considerably (FIG. 5B), which is consistent with the notion that the hypothetically pre-bound aryl group in CCVJ1 positions the nucleophile well for reaction, while longer linkers in CCVJ2/3 would engender a higher entropic penalty for reaction. When linker L1 (having no aryl group) was added to the reaction as a competing alpha nucleophile, it showed no effect at concentrations up to 500 μM, suggesting the presence of the aryl group in CCVJ1 markedly accelerates the reactivity of linker L1 (FIG. 6). Interestingly, the brightness of CCVJ2 and CCVJ3 probes was diminished compared to that of CCVJ1 (FIG. 7). To test the importance of a double-stranded DNA structure to the rate and fluorescence response, an AP-containing single-stranded DNA oligonucleotide was reacted with CCVJ1. At pH 7, CCVJ1 reacted with a second-order rate constant of 33 M⁻¹s⁻¹ with the single-stranded substrate, roughly 5-fold slower than a double-stranded hairpin with identical sequence context (FIG. 8). Furthermore, the reaction with the single-stranded DNA only yielded a 4-fold increase in fluorescence, suggesting that rigidly stacked neighboring bases (as found in double-stranded DNA) are important for constraining bond rotation in CCVJ1. These results are consistent with the hypothesis that the more rigid binding site created by the AP site in double stranded DNA is important to the observed rate acceleration.

In addition to Tris buffer, oxime formation kinetics were also measured for CCVJ1 with AP DNA in a solution of a catalytic amine buffer previously shown to accelerate oxime formation. To this end, it was employed 50 mM N,N-dimethylethylenediamine (DEDA)•HCl buffered to a pH of 7.0 in place of the above Tris buffers. Under these conditions, it was found that the rate of oxime formation proceeded yet ˜3 times faster, achieving a second-order rate constant of 440 M⁻¹s⁻¹ (FIG. 5B). Such rapid rates of oxime formation at neutral pH are unprecedented for unactivated aldehydes.

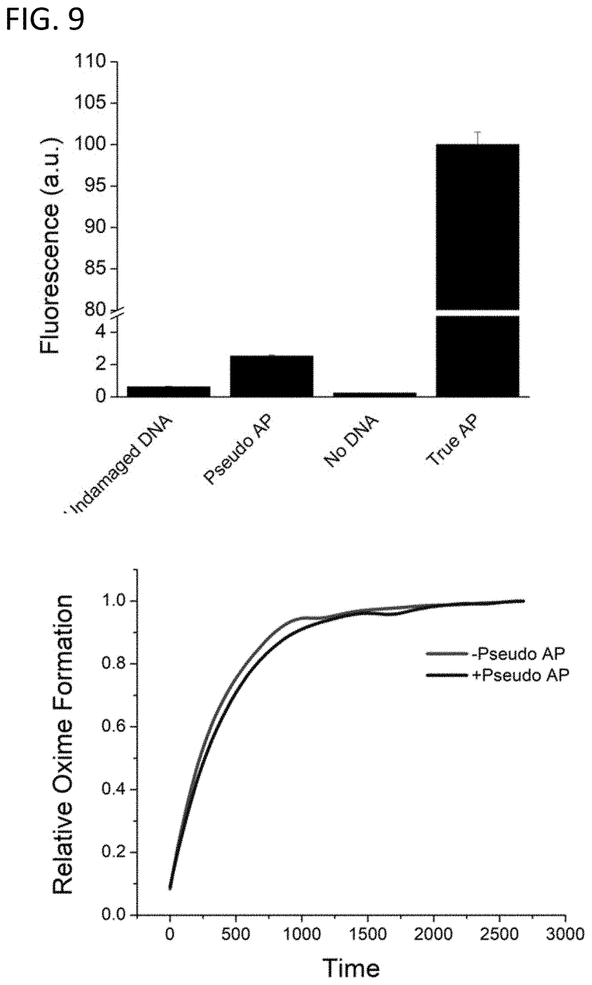

To further explore the hypothesis that the oxime rate acceleration in these AP-site DNAs was related to base stacking interactions, the fluorescence was measured of CCVJ1 in the presence and absence of a non-lesion containing DNA hairpin. It was observed a ˜2.5-fold increase in fluorescence intensity upon addition of DNA to the probe, suggesting a small but significant amount of intercalation (FIG. 9). When instead a DNA hairpin was used containing a tetrahydrofuran spacer (a pseudo AP site), which mimics an AP site without the reactive hemiacetal, it was observed a ˜10-fold increase in fluorescence over the free probe, ˜4-fold higher than with undamaged DNA, suggesting that the probe intercalates more readily into the AP site mimic than into duplex DNA alone (FIG. 9). Without a covalent attachment to the AP site, the dye-DNA interaction is hypothesized to be dynamic and flexible, given the relatively low fluorescence increase compared to the 256-fold enhancement observed upon covalent attachment after reaction with an AP site. In a separate experiment, the effect was tested of adding an equimolar amount of pseudo AP DNA to the reaction with a true abasic site DNA and observed no effect on the rate of apparent oxime formation (FIG. 9).

### Example 4: Neighboring Base Effect

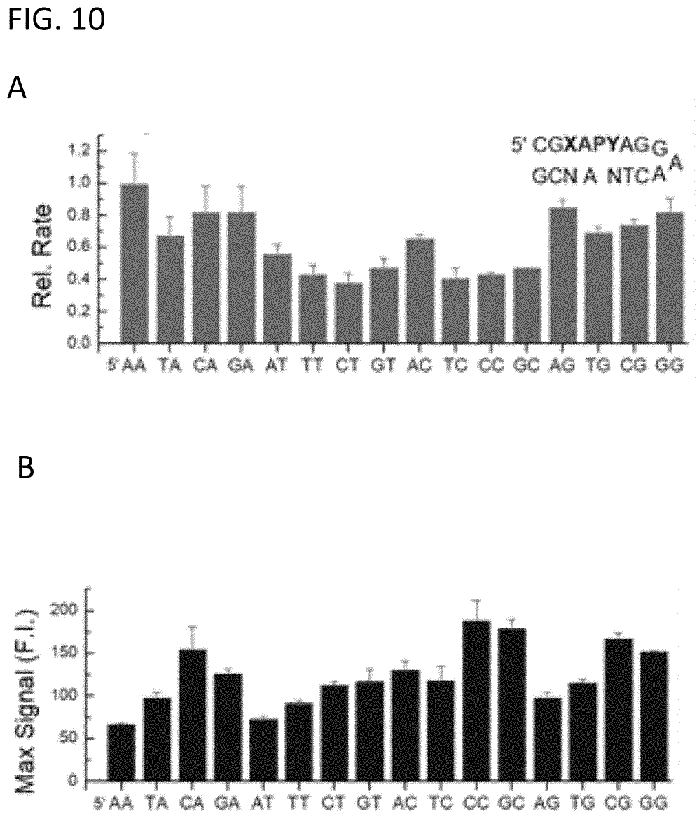

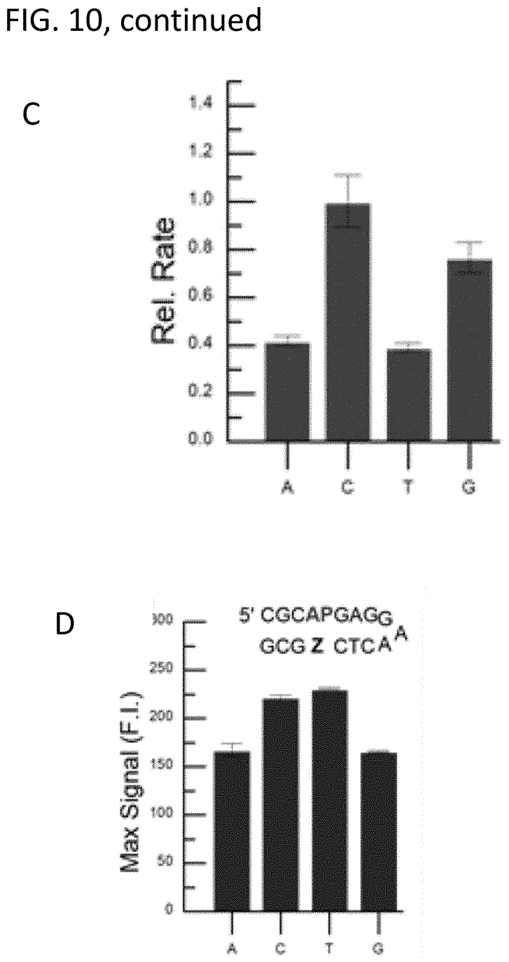

Given the well-characterized ability of DNA nucleobases to quench fluorescent species, it was sought to explore the effect that neighboring bases X and Y had on the potential light-up signal of CCVJ1 as well as on the rate of oxime formation (FIG. 10, panels A-B). To test this, CCVJ1 was reacted with a library of 16 DNA hairpins representing all possible combinations of neighboring DNA bases (FIG. 10, panels A-B). It was found that cytosine and guanine exert the least quenching effect, with combinations of X=C/G and Y=C/G yielding the greatest maximum signals, while adenine and thymine exert an apparent quenching effect that reduced signal by half. With respect to the rate of oxime formation, the greatest effect appears to be exerted by the 3′ neighboring base Y. Both guanine and adenine 3′ to the AP site consistently yielded the fastest oxime formation, with X=A, Y=A yielding the fastest rate (Relative Rate=1.0), while combinations with thymine or cytosine yielded rates about half as fast. In general, purine neighboring bases gave the highest rates, consistent with the apparent need for pre-association and stacking of the probe with the DNA to increase local concentration of the reactive aminooxy group. Purines are documented to stack more strongly with aromatic species than pyrimidines.

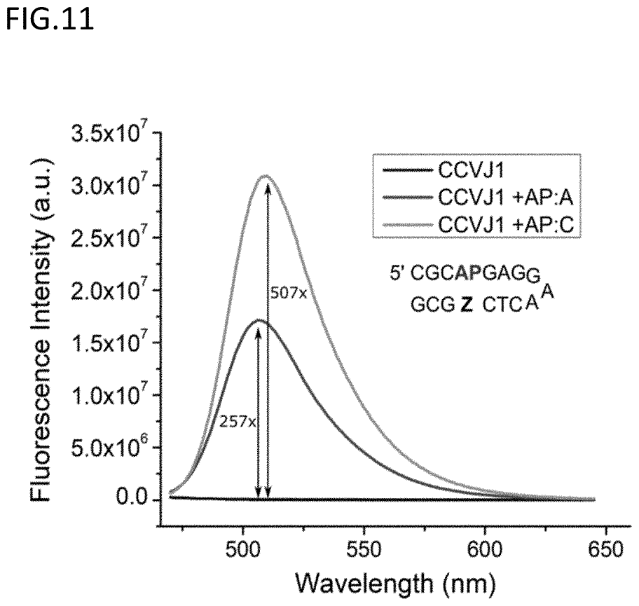

In addition to the 5′ and 3′ neighboring bases, studies were done of the effect of the orphaned base Z paired opposite the abasic site on the UBER probe system (FIG. 10, panels C-D). Using the sequence where X=C and Y=G, the identity was varied of the base Z opposite the AP site. It was found that pyrimidine bases afforded the greatest fluorescence response, increasing the overall signal by almost 50% compared to cases when Z is a purine (FIG. 10, panel C). Given that neighboring pyrimidines were shown to be quenchers of CCVJ1, the strong fluorescence signal observed when Z=C and Z=T suggests that steric occlusion by the orphaned base plays a greater role in the degree of probe light up than any electronic interactions. As a result, the smaller pyrimidine bases yielded a greater fluorescence light up response than the larger purine bases. It was also observed a strong effect by the opposing base on the rate of oxime formation (FIG. 10, panel D). Adenine, which has the strongest base stacking interactions,³² resulted in the slowest rate of oxime formation. Conversely, cytosine, which has the weakest base stacking interactions showed the fastest rate of oxime formation. This trend is in contrast to the neighboring base effect discussed above in which stronger stacking bases accelerated the rate of oxime formation. Together these results suggest that the rate of oxime formation is mediated by the favorability of CCVJ1 stacking with adjacent X and Y bases as well as the competition with base stacking of the opposing Z base. Overall, it was concluded that the optimal neighboring base combination for monitoring glycosylase activity with CCVJ1 can be attained with X=C, Y=G and Z=C which reacted with CCVJ1 at a rate of 320±13 M⁻¹s⁻¹ and afforded an overall 508-fold increase in fluorescence relative to the probe alone (FIG. 11).

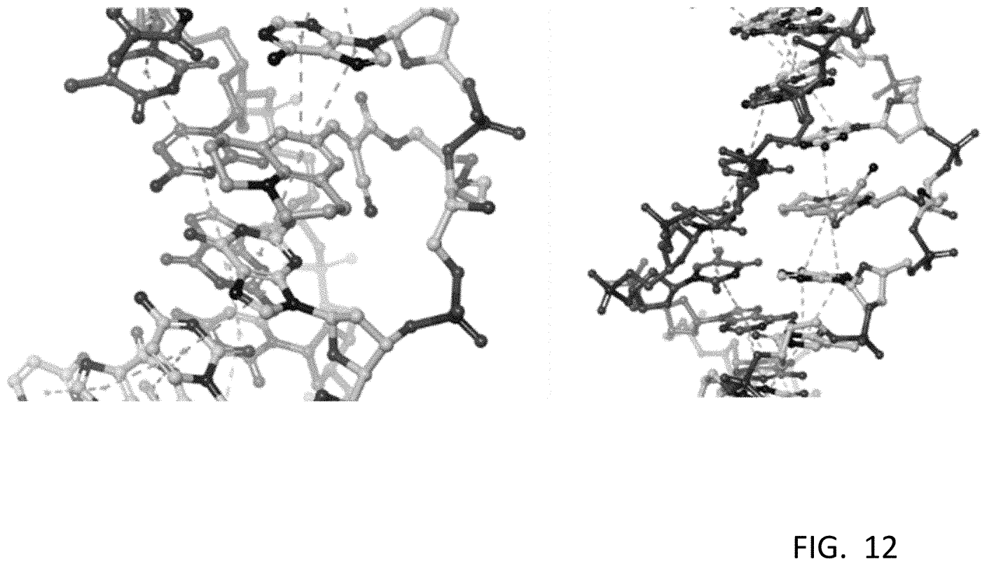

Using the experimental data obtained by our neighboring base studies, it was constructed a model of CCVJ1 bound to the AP site (FIG. 12). The resulting structure shows the dye intercalated into the AP site, and suggests that CCVJ1 may approach the AP site from the major groove with substantial overlap to the 3′ side of the dye, consistent with the fact that the 3′ neighboring base exerts the most significant effect on rate. The model suggests that the aromatic ring of CCVJ1 plausibly undergoes stacking with neighboring bases, consistent with the observed fluorescence quenching effect, while the linker protrudes into the minor groove. Additionally, the orphaned thymidine base is partially disrupted from its base stacking interactions.

### Example 5: AP Site Selectivity and Preference

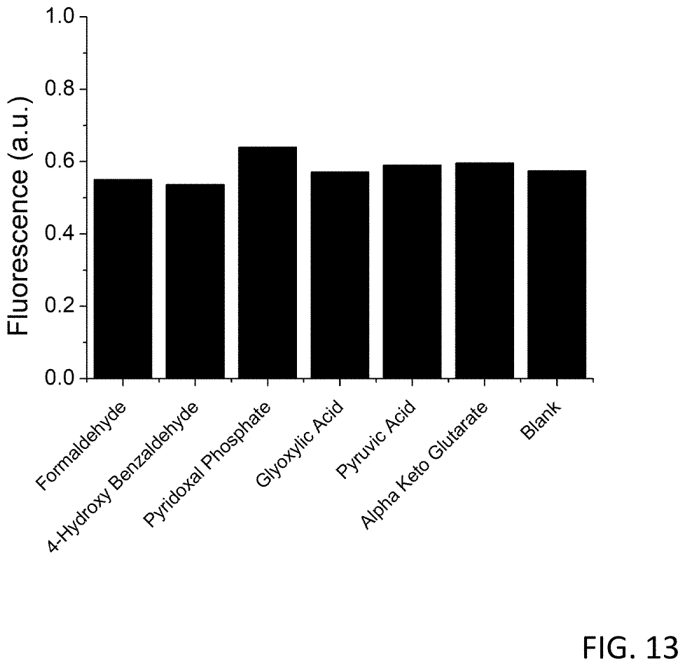

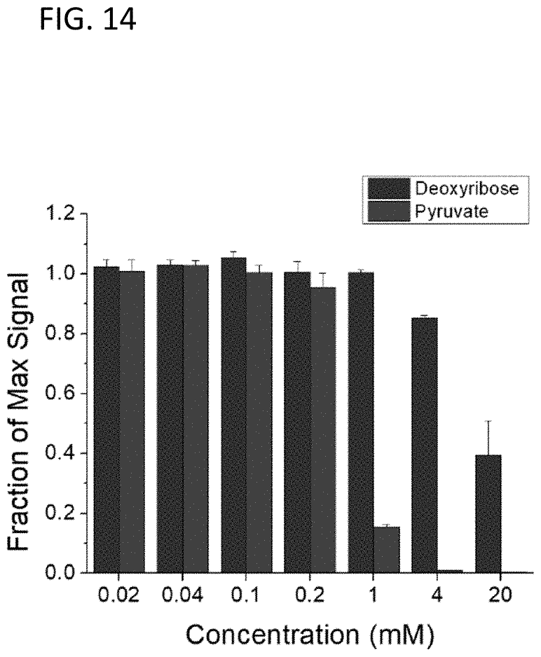

Since the probe design involves conformational restriction to induce a light-up signal, it was hypothesized that off-target oxime formation with small molecules might induce a relatively small signal relative to DNA, thus providing a measure of selectivity in signaling. To test this possibility, CCVJ1 was reacted with 500 μM of several biologically relevant ketones and aldehydes including formaldehyde, 4-hydroxybenzaldehyde, pyridoxal phosphate, glyoxylic acid, pyruvic acid and α-keto glutarate, and found that none of them generated a significant light-up response after 30 minutes of incubation, suggesting that the light-up response of this probe is highly selective for the DNA AP site (FIG. 13). Another potential aspect is that ketones or aldehydes in the environment might compete with the DNA AP site to react with the aminooxy group, thereby consuming the free probe and reducing the maximum signal. Initial testing of the reactivity of CCVJ1 with AP site-containing DNA in the presence of increasing amounts of competing 2-deoxyribose, the closest small-molecule analogue to the DNA AP site. It was found that even in the presence of 1 mM deoxyribose (200 equivalents), there was no effect on the maximum signal observed from the probe, and at 4 mM (800 equivalents) the probe still retained 85% of the signal compared to the AP DNA alone (FIG. 14). This was repeated this experiment with pyruvic acid, a highly reactive ketone and a common cellular metabolite. It was found that concentrations up to 200 μM pyruvate (40 equivalents), which is comparable to levels of pyruvate found in the cell,³³ gave no significant reduction in overall signal. These data further bolster the hypothesis that there is a pre-association between the DNA bases surrounding the AP site and the planar portion of the probe, causing the rate acceleration to be highly selective for the AP site of DNA.

### Example 6: Coupled Assay Measurements

Similar to an enzyme coupled assays, the UBER probe design generates signal via a secondary reaction which consumes the product of the first enzymatic reaction according to the following scheme.

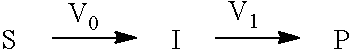

The observed signal, therefore, is equal to the rate v₁. However, unlike enzyme coupled assays, the UBER probe design does not involve enzymatic catalysis for the secondary reaction to occur at a reasonable rate. In the early phase of enzyme coupled reactions, as intermediate accumulates, the rate of v₁ increases and asymptotically approaches v₀. This relationship allows for the direct measurement of enzyme activity in a coupled assay system. The delay between the start of the reaction (t=0) and the time at which v₀≈v₁ is referred to as the lag time, tₛₛ. For a one-enzyme coupled system, McClure derived the equation for lag time which can be adapted for our system as

$${t}_{s s} = \frac{ln ( 1 - F )}{{k}_{2} * [ probe ]} \qquad ( 1 )$$

where k₂ is the second-order rate constant of oxime formation, [probe] is the concentration of UBER probe, and F1 is a constant term defining the limit at which v₀ and v₁ are defined as equal, typically 0.99. Equation (1) demonstrates that the lag time for the UBER probe coupled assay is governed solely by the concentration of probe and the second-order rate constant of oxime formation. Therefore, assay conditions can be adjusted to ensure that lag time is reasonably short by increasing probe concentration.

Given the second-order rate constants calculated above for CCVJ1 in Tris buffer and a probe concentration on the order of 10-100 μM, it was calculated that tₛₛ for an UBER probe coupled assay will be on the order of 5-50 minutes. This is in marked contrast with typical oxime k₂ rate constants of 0.01-1 M⁻¹s⁻¹ which would yield impractical tₛₛ values of 10-1000 hours unless mM concentrations of probe were used. Therefore, the ultrafast oxime formation kinetics of the UBER probe are important for the practical implementation of a coupled assay, and allow the direct measurement of enzyme activities.

### Example 7: CCVJ1 as a General DNA Glycosylase Sensor

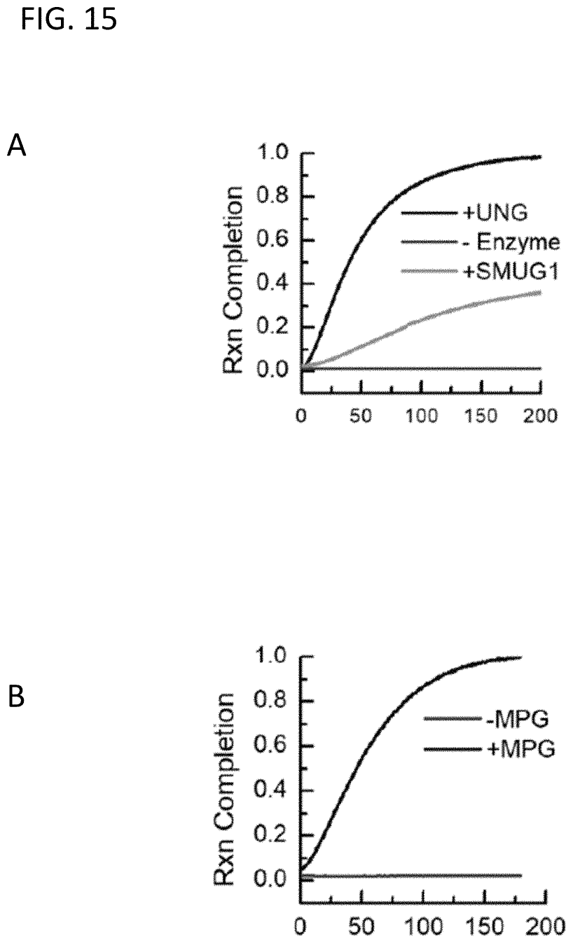

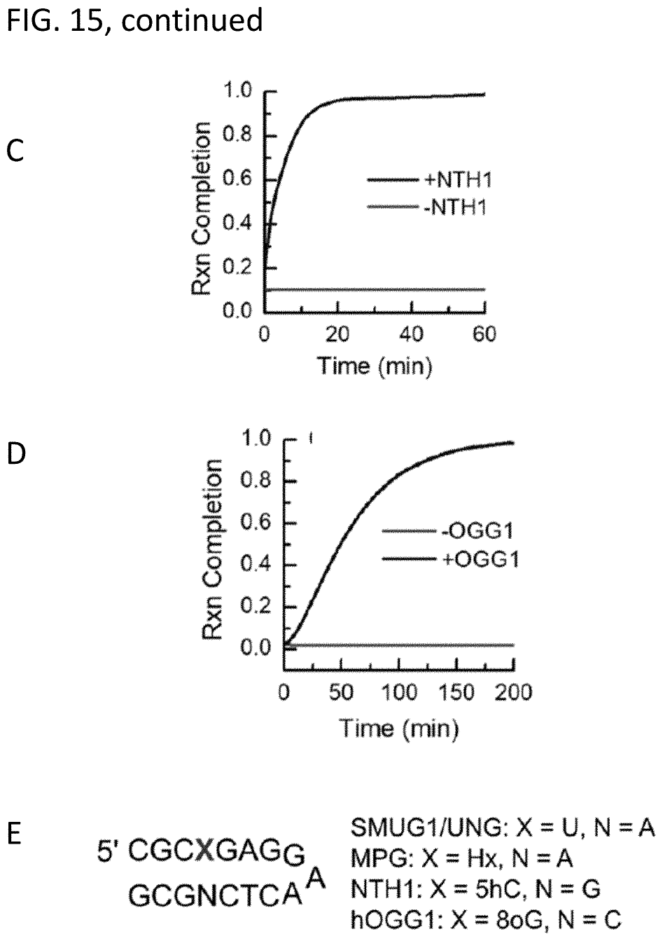

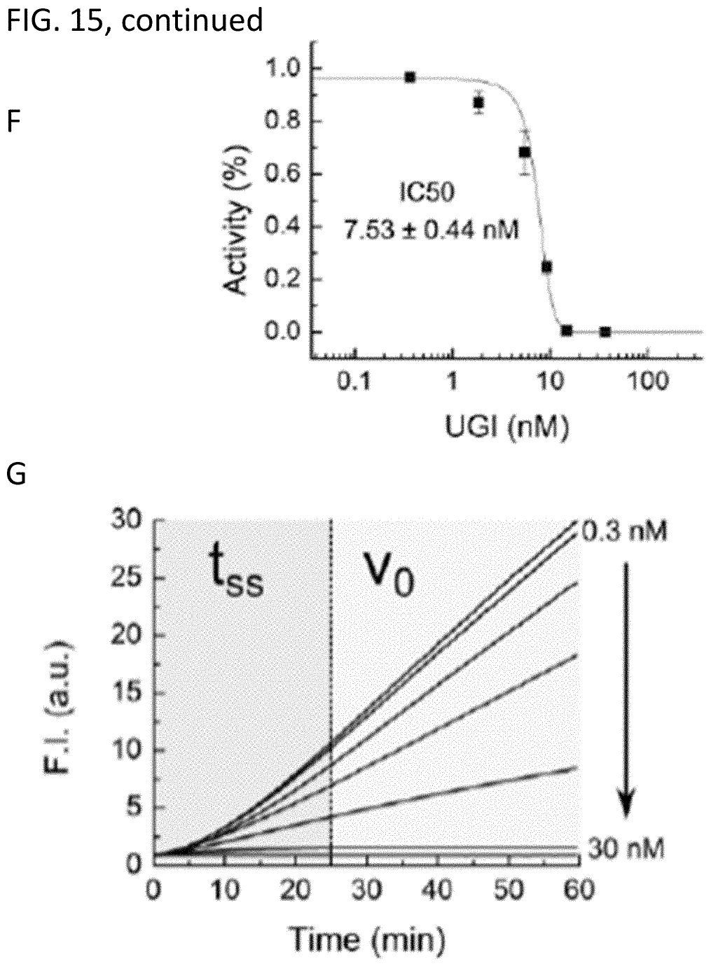

Based on the results of our neighboring base effect study, several glycosylase oligonucleotide substrates were purchased or prepared in which the identity of the base lesion X was varied to correspond to the known activities of different target glycosylases (FIG. 15, panel E). For these studies human enzymes SMUG1/UNG, OGG1, MPG and NTH1, were chosen which represent a diverse array of human glycosylases. The standard assay conditions were set as 2 μM substrate and 20 μM probe. Assays were carried out in a 60 μL volume in a 384 well format on a microplate reader using a fluorescein filter set. For each enzyme, it was possible to observe a robust fluorescence signal (FIG. 15, panels A-D). Consistent with literature measurements, it was found SMUG1 to be significantly slower than UNG on a double stranded substrate (FIG. 15, panel A).

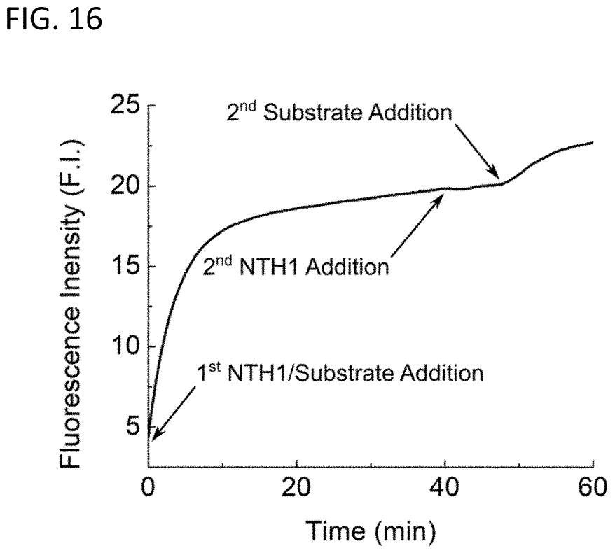

One practical consideration with the UBER probe design is the secondary lyase activity of the subset of BER enzymes that act as bifunctional glycosylases. While monofunctional glycosylases only exhibit base excision activity, bifunctional glycosylases possess a secondary strand scission or lyase activity that cleaves the DNA backbone after base excision. Since alpha nucleophiles such as alkoxyamines are known to inhibit lyase activity and prevent cleavage of the AP site, it was hypothesized that the UBER probe system could still detect bifunctional glycosylases. While lyase activity is common in bacterial glycosylases, among human glycosylases only NTH1 and NEIL1-3 possess robust lyase activity. Notably, while OGG1 is considered a bifunctional glycosylase, its lyase activity proceeds relatively slowly and is generally considered a monofunctional glycosylase in vivo. Indeed, under our assay conditions OGG1 generated signal in an equivalent manner to the monofunctional glycosylases were tested. However, when testing CCVJ1 with NTH1 using a 5-hydroxycytosine (5hC)-containing DNA hairpin, it was observed a partial lowering of signal compared to the monofunctional glycosylases, suggesting that some of the hairpin substrate is cleaved prior to reacting with CCVJ1. In some contexts, NTH1 is reported to behave as a pseudo-single-turnover enzyme which could also explain the partial loss of signal. However, subsequent additions of enzyme did not yield a fluorescence increase which rules out the pseudo-single-turnover explanation (FIG. 16). It was found that using higher concentrations of the DNA substrate (60 μM) yielded higher fluorescence signal, consistent with the hypothesis that much of the potential AP-sites are lost to lyase activity. In spite of this loss of signal, the overall fold change observed (20-fold) is still sufficient for detection and quantification of enzymatic activity.

To test the utility of the probe coupled assay for characterizing inhibitors, it was measured the IC₅₀ value of the UNG inhibitor UGI (FIG. 15, panels F-G). The resulting IC₅₀ of 7.53±0.44 nM is consistent with literature values reporting its tight 1:1 binding stoichiometry. In all cases, the z-factor of the assay was calculated to be >0.95, well above the threshold necessary for high throughput screening.

### Example 8: Assaying Substrates Generated In Situ

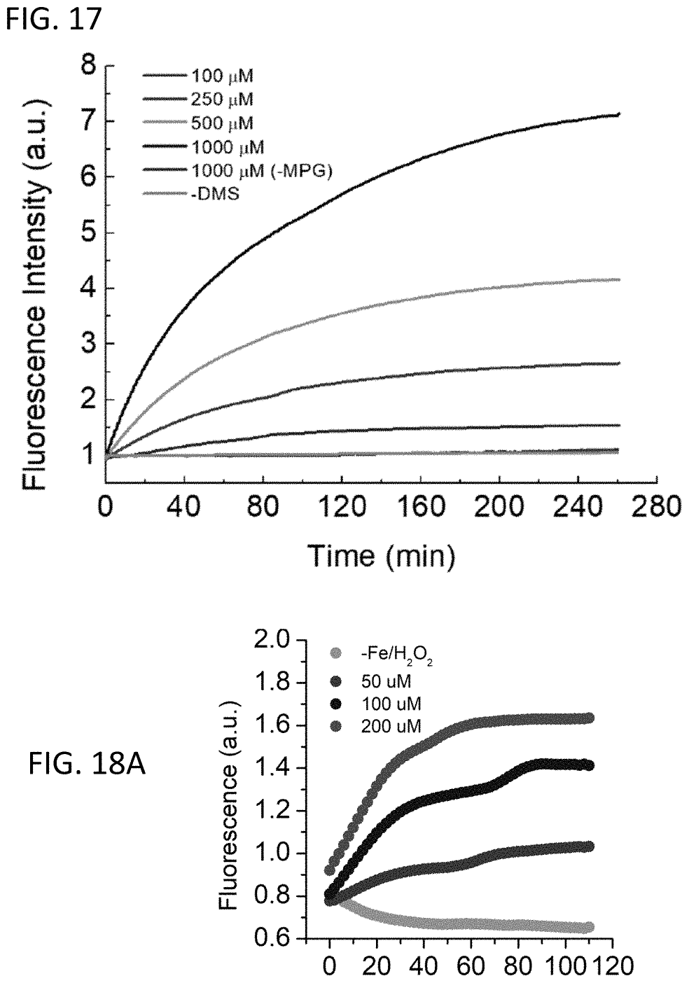

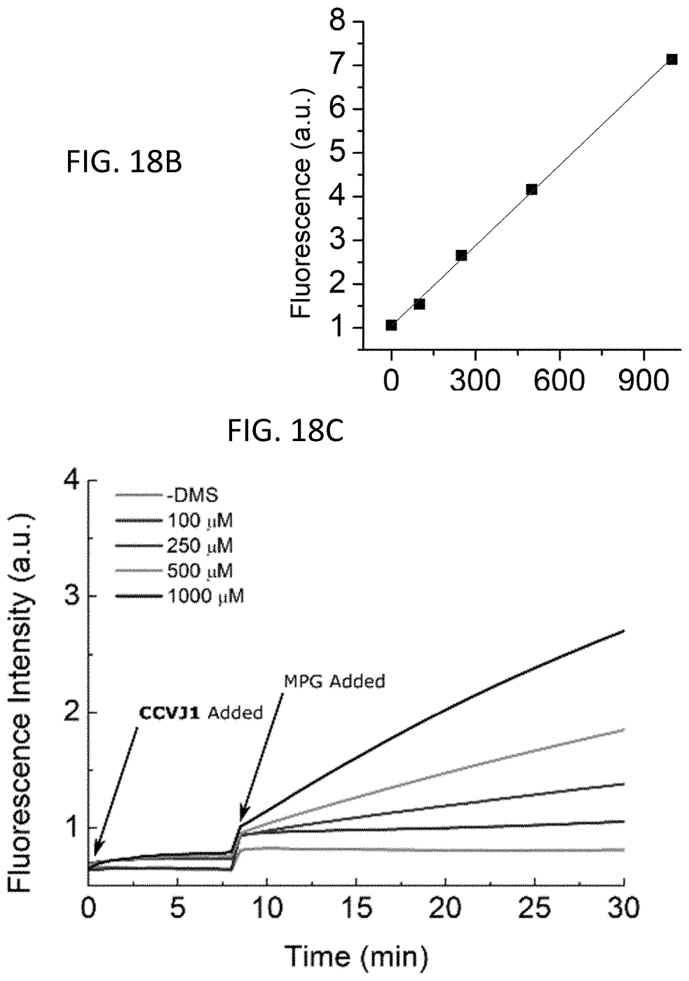

Since the probe and substrate are independent from one another, the UBER probe assay can also be implemented without synthesizing lesion-containing oligonucleotide substrates. This is particularly advantageous for cases where the DNA lesion of interest is costly or challenging to incorporate into a synthetic oligonucleotide using conventional phosphoramidite synthesis. For example, by treating calf thymus DNA (ctDNA) with increasing concentrations of the DNA alkylating compound dimethyl sulfate (DMS) for 2 hours, it was possible to generate a suitable alkylated DNA substrate for the enzyme MPG (FIG. 17). Upon treatment of 0.1 mg/mL alkylated ctDNA with 100 nM MPG in the presence of CCVJ1 (20 μM), an increase in fluorescence was observed that correlated linearly to the concentration of DMS used (FIG. 18A). Alkylated ctDNA that was treated with the highest level of DMS (1 mM) showed no increase in fluorescence when treated with probe alone, suggesting that spontaneous depurination of the alkylated ctDNA, which could lead to a false positive, occurs at a negligible rate under our assay conditions (FIG. 17). Pre-incubation of the alkylated ctDNA with CCVJ1 prior to the addition of MPG also showed stable fluorescence, providing further evidence that spontaneous depurination occurs at a rate far below background (FIG. 18C). Similarly, it was possible to produce a substrate for OGG1 by treating calf thymus DNA under oxidizing conditions with Fenton's reagent (FIG. 18B). These preliminary tests suggest that by generating suitable enzyme substrates in situ, the UBER probe can be employed with inexpensively produced, biologically derived substrates and can circumvent the need to synthesize modified oligonucleotides if desired.

### Example 9: Profiling Cellular Glycosylase Activity

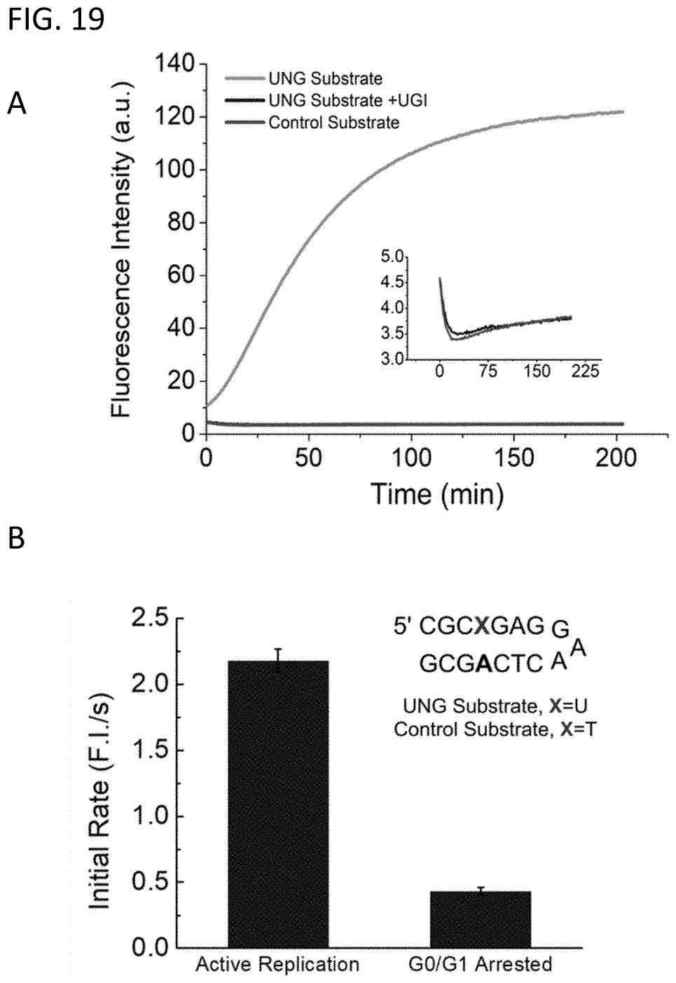

After demonstrating that the UBER probe could efficiently report on base excision activity from a variety of glycosylases in vitro, it was explored whether the probe could be employed to profile endogenous glycosylase activity in cell lysates. It was first tested whether the UBER probe's ability to report on UNG activity in whole cell lysates using the previously described UNG hairpin substrate (FIG. 19, panel A). Using 5 μM hairpin and 25 μM CCVJ1, strong, real-time fluorescence response was observed in the presence of 0.2 mg/mL crude HeLa cell lysate. The oligonucleotide substrate sequence is based on the high melting GAA sequence motif that has been shown to confer high nuclease stability in short hairpins. To confirm that the fluorescence response originated from endogenous UNG activity and not off-target binding with proteins or spontaneous depurination, lysate was treated with CCVJ1 and a control hairpin in which the lesion had been replaced by an undamaged T:A base pair. Importantly, the control hairpin yielded no fluorescence response indicating a low degree of false positive signal. Additionally, treating the lysate with 1 U/mL of inhibitor UGI completely ablated the fluorescence response, confirming the specific enzymatic origin of the light-up activity. CCVJ1 appears to represent the first fluorogenic probe to measure real-time UNG activity in cell lysates.

To further demonstrate the utility of the UBER probe, it was used to monitor changes in UNG activity at different phases of the cell cycle. Lysates were generated from HeLa cells arrested in the G0/G1 phase as well as actively dividing cells, and UNG activity was quantified by measuring initial rate velocity. A ˜5-fold increase was observed in UNG activity in actively dividing cells relative to cells arrested in the G0/G1 phase (FIG. 19, panel B), consistent with literature reports. This experiment demonstrates the ability of the UBER probe to measure dynamic changes in glycosylase expression level in response to environmental stimuli or disease states using a simple mix-and-measure format.

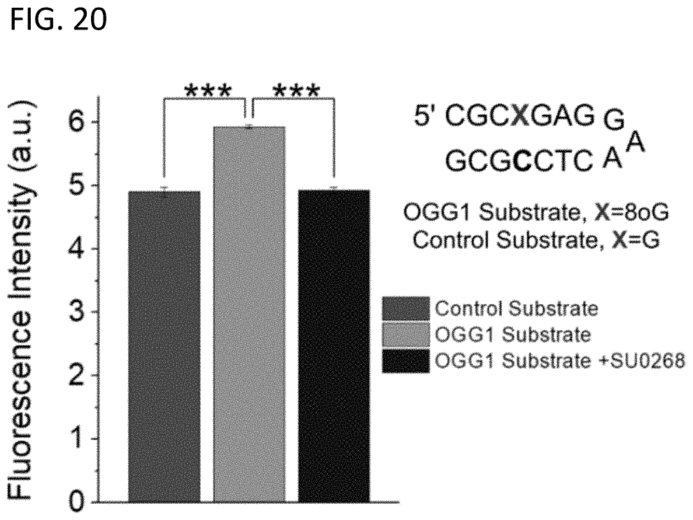

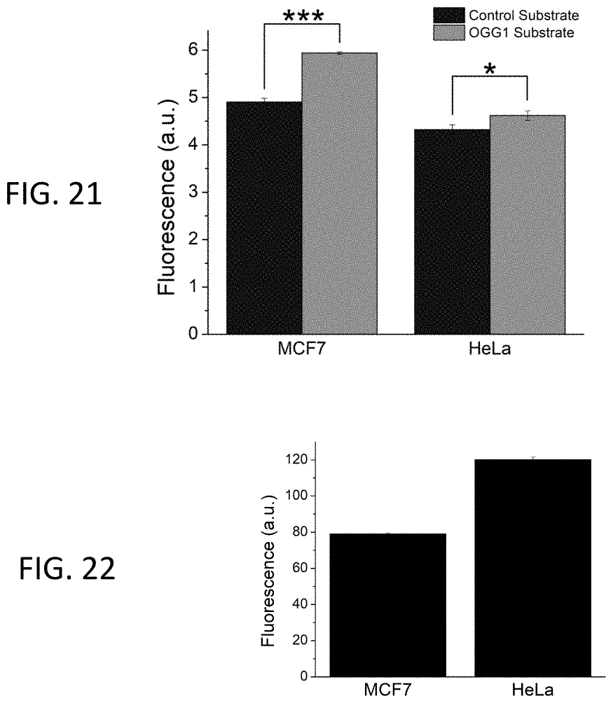

Encouraged by the results, whether the UBER probe could detect endogenous glycosylase activity was tested from a more challenging target such as OGG1. It is well established that the turnover number of UNG is quite high (˜1-10 s⁻¹)⁵ while the turnover number for many other glycosylases is quite low (0.001-0.1 s⁻¹). Additionally, most glycosylases, including OGG1, have very low basal expression levels in healthy cells. Given these facts, previous attempts to detect OGG1 activity in lysates have relied on a multistep signal amplification process or extended reaction times (24-48 hrs) to yield signal. However, it was found that after a 4 h incubation with CCVJ1 it was possible to quantify OGG1 activity in MCF7 lysates (FIG. 20). While the overall signal was appreciably lower than that observed for UNG, addition of the potent OGG1 inhibitor SU0268 completely abolished OGG1 repair activity relative to the control, demonstrating the sensitivity of CCVJ1. In this case, the majority of the background signal observed was attributed to intercalation of probe into unreacted hairpin as well as genomic DNA. In additional experiments, it was also used CCVJ1 to measure OGG1 activity in HeLa cells which demonstrated ˜3× lower OGG1 activity, consistent with literature findings of relative OGG1 activity in these two cell lines (FIG. 21). Interestingly, HeLa cells demonstrated ˜2× higher UNG activity than MCF7 cells (FIG. 22).

### Conclusions

It has been shown that the subject probes, which include a fluorophore (e.g., molecular rotor dyes) with optimized, relatively short linkers, can yield robust and rapid fluorescence signals in response to abasic sites generated during DNA base excision repair. Perhaps the most striking finding in this study is the unprecedentedly fast oxime formation reaction observed between UBER probe CCVJ1 and DNA AP-sites. Rates that are 3-4 orders of magnitude faster than standard oxime bond formation reactions at neutral pH were observed. As a bioorthogonal labeling strategy, oxime linkages have generally been criticized for suffering from slow reaction rates (˜0.001-0.01 M⁻¹s⁻¹) relative to a number of recently reported biofunctionalization reactions such as tetrazine ligations (1-100 M¹s⁻¹). Given this context, the results presented here are notable. It is worth noting that under more conventional oxime formation rates, the UBER probe design would involve days or possibly months to reach tₛₛ and would likely suffer from off-target reactivity with other aldehydes and ketones. Therefore, an important aspect of the success of the UBER probe design is its high selective and rapid oxime formation with the AP site.

The UBER probe design exhibits several significant advantages over previously reported methods for assaying DNA glycosylase activity. Traditional biochemical methods such as gel-based assays or radiation release assays have the advantage of sensitivity as well as using unmodified, native substrates. However, they are discontinuous and therefore time and labor intensive. To illustrate this point, consider a small 394-compound library screen of potential glycosylase inhibitors. Using a discontinuous method, screening each compound at a single concentration using 5 time-points to measure vᵢ would require 1,970 individual enzyme reactions to be run and quenched at the same time for each prior to running each reaction on a gel. Using a continuous fluorescence assay, the entire screen could be completed on a single microplate in a matter of hours. Molecular beacon probes of base excision, like the current probes, have the advantage of being continuous, however since they rely on strand cleavage they cannot be used with monofunctional glycosylases, which represent the majority of human glycosylases, without further downstream processing. CCVJ1, with its 250-500-fold increase in fluorescence, combines sensitivity and generalizability in a continuous assay that can be adapted for use with virtually any human glycosylase. Furthermore, the ability to monitor the dynamic response of glycosylase activity in cell lysates allows efficient glycosylase activity profiling which previously would have only been achieved through lower sensitivity fluorescence methods which could require days to develop an observable signal.

By utilizing a separate reporter molecule, the UBER probe can be paired in a coupled assay with any DNA substrate without further modification. This can allow sensing of as-yet undiscovered DNA glycosylase activities. In many cases, enzyme substrates can be purchased from commercial oligonucleotide suppliers or generated in situ from biologically derived DNA, negating the need to purchase or synthesize heavily modified oligonucleotides. Additionally, by working with native substrates rather than modified reporter oligonucleotides, the UBER probe produces no interference with native enzyme activity. Unlike previous probes that rely on secondary DNA cleavage (lyase) activity to generate signal, the UBER probe generates signal in direct proportion to base excision and requires no secondary enzyme activity. In particular, CCVJ1 produces a significantly larger fluorescence response (e.g., light-up response) than a conventional molecular beacon which must be customized for each enzyme of interest. This large fold change allows sensitive detection of DNA glycosylase activity in cell lysates in a relatively short amount of time.

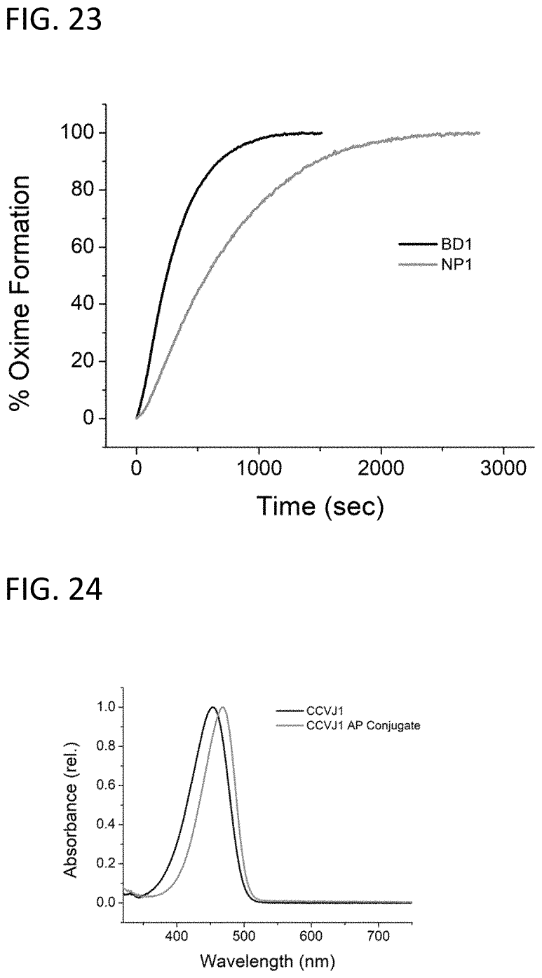

It should be noted that both the naphthalimide probes NP1 and NP2 as well as benzoxadiazole probes BD1 and BD2 also experienced accelerated rates of oxime formation and substantial fluorescence responses with AP DNA (FIG. 23), suggesting that this molecular strategy could be general and can provide different emission colors as desired (FIG. 4). Exemplary probe CCVJ1, which employs a molecular rotor dye with a short, 2-carbon linker and yields a robust >250-500-fold fluorescence light up response to AP sites. The probe design allows real-time reporting of glycosylase base excision in a simple mix-and-read format. Additionally, because of its unusual rate acceleration and light up mechanism, the UBER probe design offers very low background and low off-target light up signals, even in the presence of high concentrations of common small-molecule carbonyl compounds and the complex matrix of cellular lysates. Moreover, the synthesis of CCVJ1 is facile, proceeding in four steps with high yields. These properties make the probe a promising tool for researchers interested in studying, in principle, any DNA glycosylase either in vitro or in tissue or cell extracts.

Methodology

Instrumentation: NMR spectra were acquired on a Varian Inova 400 (400 MHz) or 500 (500 MHz) spectrometer and chemical shift reported in parts per million (6) relative to internal standard TMS (0 ppm). Small molecule mass spectra were measured on a Waters 2795 system via electrospray ionization (ESI) with a ZQ single quadrupole MS. Oligonucleotide mass spectra were acquired by MALDI-TOF using a Bruker Microflex MALDI-TOF in negative ion mode. Ultraviolet spectra were measured on a Cary 300. Fluorescence emission and excitation spectra were recorded on a Jobin Yvon-Spex Fluorolog 3 spectrometer with an external temperature controller. Fluorescence time courses were collected on a Fluoroskan Ascent Microplate Fluorometer (Thermo Fisher Scientific). Oligonucleotide concentrations were determined by UV-absorption on a NanoDrop One Microvolume UV-Vis Spectrophotometer (ThermoFisher Scientific). DNA synthesis was carried out on an Applied Biosystems 394 DNA/RNA synthesizer.

Synthesis and Chemicals: All chemicals were purchased from Acros Organics, Combi-Blocks, Sigma-Aldrich and Oakwood Chemical and used without further purification. Phosphoramidites were purchased from Glen Research. Analytical TLC was performed on ready-to-use plates with silica gel 60 (Merck, F254), Flash column chromatography was performed over Fisher Scientific silica gel (grade 60, 230-400 mesh). All reagents were weighed and handled in air and backfilled under argon at room temperature. Unless otherwise noted, all reactions were performed under an argon atmosphere.

Enzymes and Buffers: E. coli Uracil DNA Glycosylase (UDG), Methyl Purine Glycosylase (MPG) and Single-strand selective monofunctional uracil glycosylase 1 (SMUG1) were purchased from New England Biolabs. Endonuclease III-like protein 1 (NTH1) and 8-oxoguanine glycosylase (OGG1) were purchased from Novus biologics. UNG was purchased from OriGene and whole cell lysates were purchased from Santa Cruz Biotech. Buffers were prepared from stock solutions from Sigma Aldrich.

General procedure for creating AP site DNA: A deoxyuridine containing oligonucleotide (typically 5-20 μM) was treated with 10 U/mL E. coli UDG (New England Biolabs) for 10 minutes at 37° C. in buffer to create AP site containing DNA (see below for MALDI-TOF confirmation). The hairpin was then reacted with probe from a DMSO stock solution (final DMSO concentration <5%).

Procedure for assessing fluorescence response of probes: To assess the fluorescence response of the probes, 25 μM AP site containing DNA (oligo 15) was prepared in a 20 μL solution and allowed to react with 500 μM of each probe overnight. The DNA was then precipitated by the addition of 80 μL 0.33 M sodium acetate and 300 μL ethanol and centrifuged at 21,100 g for 1 hr at 4° C. The resulting DNA pellet was washed twice with 70% EtOH and re-suspended in buffer to a concentration of 2 μM. A portion of the DNA pellet was used for MALDI-TOF analysis. The emission spectra of the probe-oligonucleotide conjugate was measured and compared against a 2 μM solution of the free probe

In situ lesion formation: Calf thymus DNA (ctDNA) (Sigma Aldrich) was diluted to a concentration of 0.1 mg/mL in buffer from a 1 mg/mL stock solution in water. Fenton's reagent was generated by combining equimolar amounts of iron (II) ammonium sulfate and hydrogen peroxide at varying concentrations (50-200 μM). ctDNA was treated with increasing amounts of either dimethyl sulfate (DMS) (100-1000 μM), Fenton's reagent (50-200 μM) or a buffer control at 37° C. for 2 hr. The reaction was then quenched by the addition of 1 mM 2-mercaptoethanol and allowed to stand at room temperature for 30 minutes. CCVJ1 was added to the reaction mixture (20 μM) and 60 μL aliquots were distributed onto a 384-well microplate (60 μL). DNA repair enzymes (100 nM OGG1 or MPG) were added directly to the well and fluorescence intensity monitored for 4 hours.

UGI IC50 measurement: Initial rate velocities were measured with UNG (5 nM), Oligo 15 (20 μM) and CCVJ1 (2 μM) and increasing amounts of UGI (0.3 to 30 nM). Initial rates were calculated as the slope of the fluorescence time course following a 25 minute delay time. Reactions were performed in triplicate and the resulting IC₅₀ curve was generated by fitting the data to the Boltzmann equation in OriginPro 8.5.

Cell Growth and Lysate Preparation: HeLa cells were grown in DMEM supplemented with FBS (10%), penicillin (100 U/mL), and streptomycin (100 U/mL) in a humidified incubator at 37° C. with 5% CO₂. Cells were arrested at phase G0/G1 by serum starvation for 24 hours. To prepare lysates, cells were collected in PBS by scraping and the protocol for the CellLytic™ NuCLEAR Extraction Kit (Sigma Aldrich) was used with Roche complete mini EDTA-free protease inhibitor tablets. Briefly, cells were grown to ˜90% confluency and harvested by scraping (˜5×10⁷). Cells were rinsed twice with cold PBS and swelled in hypotonic lysis buffer for 15 minutes on ice (10 mM HEPES, pH 7.9, with 1.5 mM MgCl₂, 10 mM NaCl, 0.1 M DTT and 1× protease inhibitor). Cells were lysed by repeated passage through a 25-gauge needle and the cytosolic fraction collected. Nuclear proteins were extracted from the nuclear pellet by shaking with a high salt nuclear extraction buffer for 60 minutes (20 mM HEPES, pH 7.9, with 1.5 mM MgCl₂, 420 mM NaCl, 0.1 M DTT, 25% glycerol and 1× protease inhibitor). The nuclear and cytosolic fractions were then combined and total protein was determined by Bradford assay.

Cell Lysate Experiments: HeLa or MCF-7 whole cell lysates (Santa Cruz Biotech) were used without further preparation. To assess enzymatic activity, lysates were diluted to 0.2 mg/mL in buffer (50 mM Tris buffer pH 7, 100 mM NaCl) along with CCVJ1 (25 μM) and the appropriate oligo substrate (5 μM) to a volume of 60 μL in a 384-well plate. Fluorescence intensity was monitored over the course of 4 hours at 37° C.

Fluorescence time courses: Unless otherwise stated, fluorescence time courses were collected on a Fluoroskan Ascent Microplate Fluorometer (Thermo Fisher Scientific) with black, non-binding 384 well plates (Greiner) at a reaction volume of 60 μL with 50 mM Tris buffer pH 7 (ionic strength adjusted to 100 mM with NaCl) with a fluorescein filter set (Ex. 485, Em. 538).

Molecular modeling: Molecular modeling was carried out using the Maestro 12.0 software package (Schrödinger, LLC).

List of Oligonucleotides

Unless otherwise stated, all oligonucleotides where purchased from Integrated DNA Technologies (IDT) and used without further purification. Oligonucleotides prepared in house were synthesized on an ABI 394 instrument using phosphoramidites purchased from Glen Research and purified using a Glen Pak cartridge. All sequences are comprised entirely of DNA. U=deoxyuridine, Hx=Deoxyinosine, S=tetrahydrofuran spacer, 5hC=5-hydroxycytidine, 8oG=8-oxoguanidine

TABLE 3
Oligonucleotides used.

| Oligo | | | |
|---|---|---|---|
| # | Source | Name | Sequence (5′->3′) |
| 1 | IDT | Neighbor AA | CGAUAAGGAACTTATCG |
| 2 | IDT | Neighbor TA | CGTUAAGGAACTTAACG |
| 3 | IDT | Neighbor CA | CGCUAAGGAACTTAGCG |
| 4 | IDT | Neighbor GA | CGGUAAGGAACTTACCG |
| 5 | IDT | Neighbor AT | CGAUTAGGAACTAATCG |
| 6 | IDT | Neighbor TT | CGTUTAGGAACTAAACG |
| 7 | IDT | Neighbor CT | CGCUTAGGAACTAAGCG |
| 8 | IDT | Neighbor GT | CGGUTAGGAACTAACCG |
| 9 | IDT | Neighbor AC | CGAUCAGGAACTGATCG |
| 10 | IDT | Neighbor TC | CGTUCAGGAACTGAACG |
| 11 | IDT | Neighbor CC | CGCUCAGGAACTGAGCG |
| 12 | IDT | Neighbor GC | CGGUCAGGAACTGACCG |
| 13 | IDT | Neighbor AG | CGAUGAGGAACTCATCG |
| 14 | IDT | Neighbor TG | CGTUGAGGAACTCAACG |
| 15 | IDT | Neighbor CG | CGCUGAGGAACTCAGCG |
| 16 | IDT | Neighbor GG | CGGUGAGGAACTCACCG |
| 17 | IDT | Deoxyinosine | CGCHxGAGGAACTCAGCG |
| 18 | IDT | ssDNA | CGCUGAGGA |
| 19 | IDT | Pseudo AP | CGCSGAGGAACTCAGCG |
| 20 | IDT | Control | CGCTGAGGAACTCAGCG |
| 21 | In House | NTH1 | CGC5hCGAGGAACTCGGCG |
| 22 | In House | OGG1 | CGC8oGGAGGAACTCCGCG |

Derivation of Tₛₛ equation (adapted from McClure)

The reaction scheme for a coupled reaction can be written as

Or for our purposes

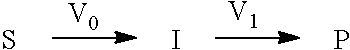

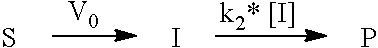

Where v₀ is the enzyme velocity and

k₂=kₚ*[probe]

Where kₚ represents the second order rate constant of oxime formation. Given a sufficiently large excess of probe is used relative to the substrate, the value of k₂ is assumed to be a constant.

At any given moment the rate at which [I] is changing may be expressed as

$$\frac{d [ I ]}{d t} = {v}_{0} - {k}_{2} [ I ]$$

When t=0 the concentration of [I]=0. As the reaction progresses, the concentration of I increases causing the rate v₁ to increase as well. Eventually, the rate of v₁ will asymptotically approach the rate of v₀ and at time infinity they will become equal. When the rate v₀≈v₁, the value of d[I]/dt will be zero and the concentration of I will reach a steady state. Setting d[I]/dt to zero, we solve for [I]ₛₛ

$${[ I ]}_{s s} = \frac{{v}_{0}}{{k}_{2}}$$

The rate equation given above can be integrated as

$$[ I ] = \frac{{v}_{0}}{{k}_{2}} ( 1 - {e}^{- {k}_{2} t} )$$

Or rearranged in terms of t as

$$ln ( 1 - \frac{{k}_{2}}{{v}_{0}} [ I ] ) = - {k}_{2} t$$

To solve for the delay time tₛₛ when steady state will be achieved, we must first define the point at which we will consider the reaction to be in steady state. As pointed out above, the value of [I] will approach [I]ₛₛ asymptotically and requires infinite time to reach [I]ₛₛ. Therefore we must choose some fraction F of [I]ₛₛ at which point the concentrations are deemed sufficiently close. Literature convention has defined the value of F as 0.99.^{7,8} Therefore, to solve for the delay time tₛₛ, we solve the integrated equation above in terms of time, substituting the term F*[I]ₛₛ for [I]

$$ln \frac{( 1 - ( \frac{{k}_{2}}{{v}_{0}} F * {[ I ]}_{s s} ) )}{- {k}_{2}} = {t}_{s s}$$

By substituting the value

$${[ I ]}_{s s} = \frac{{v}_{0}}{{k}_{2}}$$

We get the final equation for tₛₛ as

$${t}_{s s} = - \frac{ln ( 1 - F )}{{k}_{2}} OR {t}_{s s} = - \frac{ln ( 1 - 0.99 )}{{k}_{p} [ probe ]}$$

### FIRST SET OF REFERENCES

(1) Chatterjee, N.; Walker, G. C. Mechanisms of DNA Damage, Repair, and Mutagenesis. Environ. Mol. Mutagen. 2017, 58 (5), 235-263.
(2) Ciccia, A.; Elledge, S. J. The DNA Damage Response: Making It Safe to Play with Knives. Mol. Cell 2010, 40 (2), 179-204.
(3) Jacobs, A. L.; Schaer, P. DNA Glycosylases: In DNA Repair and Beyond. Chromosoma 2012, 121 (1), 1-20.
(4) David, S. S.; O'Shea, V. L.; Kundu, S. Base-Excision Repair of Oxidative DNA Damage. Nature 2007, 447 (7147), 941-950.
(5) Krokan, H. E.; Bjørås, M. Base Excision Repair. Cold Spring Harb. Perspect. Biol. 2013, 5 (4), a012583.
(6) David, S. S.; Wiliams, S. D. Chemistry of Glycosylases and Endonucleases Involved in Base-Excision Repair. Chem. Rev. 1998, 98 (3), 1221-1261.
(7) Lu, R.; Nash, H. M.; Verdine, G. L. A Mammalian DNA Repair Enzyme That Excises Oxidatively Damaged Guanines Maps to a Locus Frequently Lost in Lung Cancer. Curr. Biol. 1997, 7 (6), 397-407.
(8) Tchou, J.; Kasai, H.; Shibutani, S.; Chung, M.; Laval, J.; Groll-man, A.; Nishimura, S. 8-Oxoguanine (8-Hydroxyguanine) Dna Glyco-sylase and Its Substrate-Specificity. Proc. Natl. Acad. Sci. U.S.A 1991, 88 (11), 4690-4694.
(9) Krokan, H. E.; Drablos, F.; Slupphaug, G. Uracil in DNA-Oc-currence, Consequences and Repair. Oncogene 2002, 21 (58), 8935-8948.
(10) Dizdaroglu, M. Oxidatively Induced DNA Damage and Its Repair in Cancer. Mutat. Res. Mutat. Res. 2015, 763, 212-245.
(11) A-Tassan, N.; Chmiel, N. H.; Maynard, J.; Fleming, N.; Liv-ingston, A. L.; Williams, G. T.; Hodges, A. K.; Davies, D. R.; David, S. S.; Sampson, J. R.; Cheadle, J. R. Inherited Variants of MYH Associated with Somatic G:C->T:A Mutations in Colorectal Tumors. Nat. Genet. 2002, 30 (2), 227-232.
(12) Liu, C.; Tu, Y.; Yuan, J.; Mao, X.; He, S.; Wang, L.; Fu, G.; Zong, J.; Zhang, Y. Aberrant Expression of N-Methylpurine-DNA Glycosyl-ase Influences Patient Survival in Malignant Gliomas. J. Biomed. Bio-technol. 2012, 2012.
(13) Visnes, T.; Grube, M.; Hanna, B. M. F.; Benitez-Buelga, C.; Cázares-Körner, A.; Helleday, T. Targeting BER Enzymes in Cancer Therapy. DNA Repair 2018, 71, 118-126.
(14) Visnes, T.; Cázares-Körner, A.; Hao, W.; Wallner, O.; Masuy-er, G.; Loseva, O.; Mortusewicz, O.; Wiita, E.; Sarno, A.; Manoilov, A.; Astorga-Wells, J.; Jemth, A.-S.; Pan, L.; Sanjiv, K.; Karsten, S.; Gokturk, C.; Grube, M.; Homan, E. J.; Hanna, B. M. F.; Paulin, C. B. J.; Pham, T.; Rasti, A.; Berglund, U. W.; Nicolai, C. von; Benitez-Buelga, C.; Kool-meister, T.; Ivanic, D.; Iliev, P.; Scobie, M.; Krokan, H. E.; Baranczewski, P.; Artursson, P.; Altun, M.; Jensen, A. J.; Kalderen, C.; Ba, X.; Zubarev, R. A.; Stenmark, P.; Boldogh, I.; Helleday, T. Small-Molecule Inhibitor of OGG1 Suppresses Proinflammatory Gene Ex-pression and Inflammation. Science 2018, 362 (6416), 834-839.
(15) He, Y.-F.; Li, B.-Z.; Li, Z.; Liu, P.; Wang, Y.; Tang, Q.; Ding, J.; Jia, Y.; Chen, Z.; Li, L.; Sun, Y.; Li, X.; Dai, Q.; Song, C.-X.; Zhang, K.; He, C.; Xu, G.-L. Tet-Mediated Formation of 5-Carboxylcytosine and Its Excision by TDG in Mammalian DNA. Science 2011, 333 (6047), 1303-1307.
(16) Wilson, D. L.; Kool, E. T. Fluorescent Probes of DNA Repair. ACS Chem. Biol. 2018, 13 (7), 1721-1733.
(17) Lee, C. Y.; Park, K. S.; Park, H. G. Pyrrolo-DC Modified Duplex DNA as a Novel Probe for the Sensitive Assay of Base Excision Repair Enzyme Activity. Biosens. Bioelectron. 2017, 98, 210-214.
(18) Yu, W.-T.; Wu, T.-W.; Huang, C.-L.; Chen, I.-C.; Tan, K.-T. Pro-tein Sensing in Living Cells by Molecular Rotor-Based Fluorescence-Switchable Chemical Probes. Chem. Sci. 2015, 7 (1), 301-307.
(19) Liu, Y.; Miao, K.; Dunham, N. P.; Liu, H.; Fares, M.; Boal, A. K.; Li, X.; Zhang, X. The Cation-π Interaction Enables a Halo-Tag Fluoro-genic Probe for Fast No-Wash Live Cell Imaging and Gel-Free Protein Quantification. Biochemistry 2017, 56 (11), 1585-1595.
(20) Clark, S. A.; Singh, V.; Vega Mendoza, D.; Margolin, W.; Kool, E. T. Light-Up “Channel Dyes” for Haloalkane-Based Protein Labeling in Vitro and in Bacterial Cells. Bioconjug. Chem. 2016, 27 (12), 2839-2843.
(21) Kubo, K.; Asaeda, A.; Takamori, Y.; Ide, H. Quantitation of Abasic Sites in Mammalian-Cells by the Aldehyde Reactive Probe. J. Cell. Biochem. 1995, 280-280.
(22) Bennett, S. E.; Kitner, J. Characterization of the Aldehyde Reactive Probe Reaction with AP-Sites in DNA: Influence of AP-Lyase on Adduct Stability. Nucleosides Nucleotides Nucleic Acids 2006, 25 (7), 823-842.
(23) Condie, A. G.; Yan, Y.; Gerson, S. L.; Wang, Y. A Fluorescent Probe to Measure DNA Damage and Repair. PLOS ONE 2015, 10 (8), e0131330.
(24) Boturyn, D.; Boudali, A.; Constant, J.-F.; Defrancq, E.; Lhomme, J. Synthesis of Fluorescent Probes for the Detection of Abasic Sites in DNA. Tetrahedron 1997, 53 (15), 5485-5492.
(25) Carrasco, M. R.; Alvarado, C. I.; Dashner, S. T.; Wong, A. J.; Wong, M. A. Synthesis of Aminooxy and N-Alkylaminooxy Amines for Use in Bioconjugation. J. Org. Chem. 2010, 75 (16), 5757-5759.
(26) Kömel, D. K.; Kool, E. T. Oximes and Hydrazones in Biocon-jugation: Mechanism and Catalysis. Chem. Rev. 2017, 117 (15), 10358-10376.
(27) Liu, X.; Qiao, Q.; Tian, W.; Liu, W.; Chen, J.; Lang, M. J.; Xu, Z. Aziridinyl Fluorophores Demonstrate Bright Fluorescence and Su-perior Photostability by Effectively Inhibiting Twisted Intramolecu-lar Charge Transfer. J. Am. Chem. Soc. 2016, 138 (22), 6960-6963.
(28) Liu, T.-K.; Hsieh, P.-Y.; Zhuang, Y.-D.; Hsia, C.-Y.; Huang, C.-L.; Lai, H.-P.; Lin, H.-S.; Chen, I.-C.; Hsu, H.-Y.; Tan, K.-T. A Rapid SNAP-Tag Fluorogenic Probe Based on an Environment-Sensitive Fluoro-phore for No-Wash Live Cell Imaging. ACS Chem. Biol. 2014, 9 (10),2359-2365.
(29) Kojima, N.; Takebayashi, T.; Mikami, A.; Ohtsuka, E.; Ko-matsu, Y. Construction of Highly Reactive Probes for Abasic Site De-tection by Introduction of an Aromatic and a Guanidine Residue into an Aminooxy Group. J. Am. Chem. Soc. 2009, 131 (37), 13208-13209.
(30) Larsen, D.; Kietrys, A. M.; Clark, S. A.; Park, H. S.; Ekebergh, A.; Kool, E. T. Exceptionally Rapid Oxime and Hydrazone Formation Promoted by Catalytic Amine Buffers with Low Toxicity. Chem. Sci. 2018, 9 (23), 5252-5259.
(31) Wilson, J. N.; Cho, Y.; Tan, S.; Cuppoletti, A.; Kool, E. T. Quenching of Fluorescent Nucleobases by Neighboring DNA: The “Insulator” Concept. ChemBioChem 2008, 9 (2), 279-285.
(32) Guckian, K. M.; Schweitzer, B. A.; Ren, R. X.-F.; Sheils, C. J.; Tahmassebi, D. C.; Kool, E. T. Factors Contributing to Aromatic Stack-ing in Water: Evaluation in the Context of DNA. J. Am. Chem. Soc. 2000, 122 (10), 2213-2222.
(33) Zhu, A.; Romero, R.; Petty, H. R. A Sensitive Fluorimetric Assay for Pyruvate. Anal. Biochem. 2010, 396 (1), 146-151.
(34) McClure, W. R. Kinetic Analysis of Coupled Enzyme Assays. Biochemistry 1969, 8 (7), 2782-2786.
(35) Taverna, P.; Liu, L.; Hwang, H.-S.; Hanson, A. J.; Kinsella, T. J.; Gerson, S. L. Methoxyamine Potentiates DNA Single Strand Breaks and Double Strand Breaks Induced by Temozolomide in Colon Cancer Cells. Mutat. Res. Repair 2001, 485 (4), 269-281.
(36) Dalhus, B.; Forsbring, M.; Helle, I. H.; Vik, E. S.; Forstrom, R. J.; Backe, P. H.; Alseth, I.; Bjorbs, M. Separation-of-Function Mutants Unravel the Dual-Reaction Mode of Human 8-Oxoguanine DNA Gly-cosylase. Structure 2011, 19 (1), 117-127.
(37) Marenstein, D. R.; Chan, M. K.; Altamirano, A.; Basu, A. K.; Boorstein, R. J.; Cunningham, R. P.; Teebor, G. W. Substrate Specificity of Human Endonuclease III (HNTH1) EFFECT OF HUMAN APE1 ON HNTH1 ACTIVITY. J. Biol. Chem. 2003, 278 (11), 9005-9012.
(38) Mol, C. D.; Arvai, A. S.; Sanderson, R. J.; Slupphaug, G.; Kavli, B.; Krokan, H. E.; Mosbaugh, D. W.; Tainer, J. A. Crystal Structure of Human Uracil-DNA Glycosylase in Complex with a Protein Inhibitor: Protein Mimicry of DNA. Cell 1995, 82 (5), 701-708.
(39) Bennett, S. E.; Mosbaugh, D. W. Characterization of the Escherichia Coli Uracil-DNA Glycosylase.Inhibitor Protein Complex. J. Biol. Chem. 1992, 267 (31), 22512-22521.
(40) Jollès, B.; Réfrégiers, M.; Laigle, A. Opening of the Extraordi-narily Stable Mini-Hairpin d(GCGAAGC). Nucleic Acids Res. 1997, 25 (22), 4608-4613.
(41) Haug, T.; Skorpen, F.; Aas, P. A.; Malm, V.; Skjelbred, C.; Krokan, H. E. Regulation of Expression of Nuclear and Mitochondrial Forms of Human Uracil-DNA Glycosylase. Nucleic Acids Res. 1998, 26 (6), 1449-1457.
(42) Schermerhorn, K. M.; Delaney, S. A Chemical and Kinetic Perspective on Base Excision Repair of DNA. Acc. Chem. Res. 2014, 47 (4), 1238-1246.
(43) Chen, C.; Zhou, D.; Tang, H.; Liang, M.; Jiang, J. A Sensitive, Homogeneous Fluorescence Assay for Detection of Thymine DNA Glycosylase Activity Based on Exonuclease-Mediated Amplification. Chem. Commun. 2013, 49 (52), 5874-5876.
(44) Edwards, S. K.; Ono, T.; Wang, S.; Jiang, W.; Franzini, R. M.; Jung, J. W.; Chan, K. M.; Kool, E. T. In Vitro Fluorogenic Real-Time As-say of the Repair of Oxidative DNA Damage. ChemBioChem 2015, 16 (11), 1637-1646.
(45) Tahara, Y.; Auld, D.; Ji, D.; Beharry, A. A.; Kietrys, A. M.; Wil-son, D. L.; Jimenez, M.; King, D.; Nguyen, Z.; Kool, E. T. Potent and Selec-tive Inhibitors of 8-Oxoguanine DNA Glycosylase. J. Am. Chem. Soc. 2018, 140 (6), 2105-2114.
(46) Lindahl, T.; Nyberg, B. Rate of Depurination of Native De-oxyribonucleic Acid. Biochemistry 1972, 11 (19), 3610-3618.
(47) Klungland, A.; Lindahl, T. Second Pathway for Completion of Human DNA Base Excision-Repair: Reconstitution with Purified Proteins and Requirement for DNase IV (FEN1). EMBO J. 1997, 16 (11), 3341-3348.

### SECOND SET OF REFERENCES

(1) Carrasco, M. R.; Alvarado, C. I.; Dashner, S. T.; Wong, A. J.; Wong, M. A. Synthesis of Aminooxy and N-Alkylaminooxy Amines for Use in Bioconjugation. J. Org. Chem. 2010, 75 (16), 5757-5759.
(2) Rumble, C.; Rich, K.; He, G.; Maroncelli, M. CCVJ Is Not a Simple Rotor Probe. J. Phys. Chem. A 2012, 116 (44), 10786-10792.
(3) Lee, M. H.; Park, N.; Yi, C.; Han, J. H.; Hong, J. H.; Kim, K. P.; Kang, D. H.; Sessler, J. L.; Kang, C.; Kim, J. S. Mitochondria-Immobilized PH-Sensitive Off-On Fluorescent Probe. J. Am. Chem. Soc. 2014, 136 (40), 14136-14142.
(4) Pagano, M.; Castagnolo, D.; Bernardini, M.; Fallacara, A. L.; Laurenzana, I.; Deodato, D.; Kessler, U.; Pilger, B.; Stergiou, L.; Strunze, S.; Tintori, C.; Botta, M. The Fight against the Influenza A Virus H1N1: Synthesis, Molecular Modeling, and Biological Evaluation of Benzofurazan Derivatives as Viral RNA Polymerase Inhibitors. ChemMedChem 2014, 9 (1), 129-150.
(5) Larsen, D.; Pittelkow, M.; Karmakar, S.; Kool, E. T. New Organocatalyst Scaffolds with High Activity in Promoting Hydrazone and Oxime Formation at Neutral PH. Org. Lett. 2015, 17 (2), 274-277.
(6) Bignon, E.; Gattuso, H.; Morell, C.; Dehez, F.; Georgakilas, A. G.; Monari, A.; Dumont, E. Correlation of Bistranded Clustered Abasic DNA Lesion Processing with Structural and Dynamic DNA Helix Distortion. Nucleic Acids Res. 2016, 44 (18), 8588-8599.
(7) McClure, W. R. Kinetic Analysis of Coupled Enzyme Assays. Biochemistry 1969, 8 (7), 2782-2786.
(8) Brooks, S. P. J.; Suelter, C. H. Practical Aspects of Coupling Enzyme Theory. Anal. Biochem. 1989, 176 (1), 1-14.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A probe of formula (I):
A-L-Y
wherein:
A is a fluorophore;
L is a linker or a bond; and
Y is an alpha nucleophile, wherein the probe is of any one of formulae (IA)-(IC): 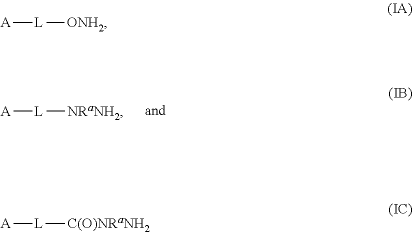
wherein:
R^{a} is selected from hydrogen, alkyl or substituted alkyl.

2. (canceled)

3. The probe of claim 1, wherein the fluorophore is a twisted intramolecular charge transfer (TICT) compound or a molecular rotor.

4. The probe of claim 1, wherein the fluorophore A is selected from a naphthalimide compound, a 9-(2-carboxy-2-cyanovinyl)julolidine (CCVJ) compound, a benzophenoxazinone (e.g., Nile Red), a benzoxadiazole, a styrylpyridinium, a stilbene, a cinnamonitrile compound, and a thiazole orange compound.

5. The probe of claim 4, wherein the fluorophore A is described by any of formulae (II-A)-(II-L): 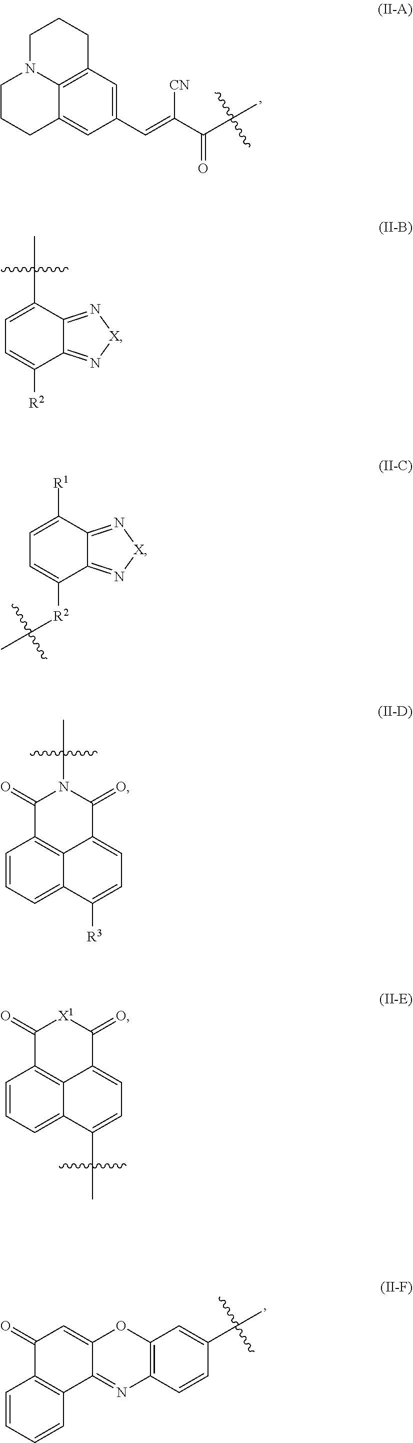 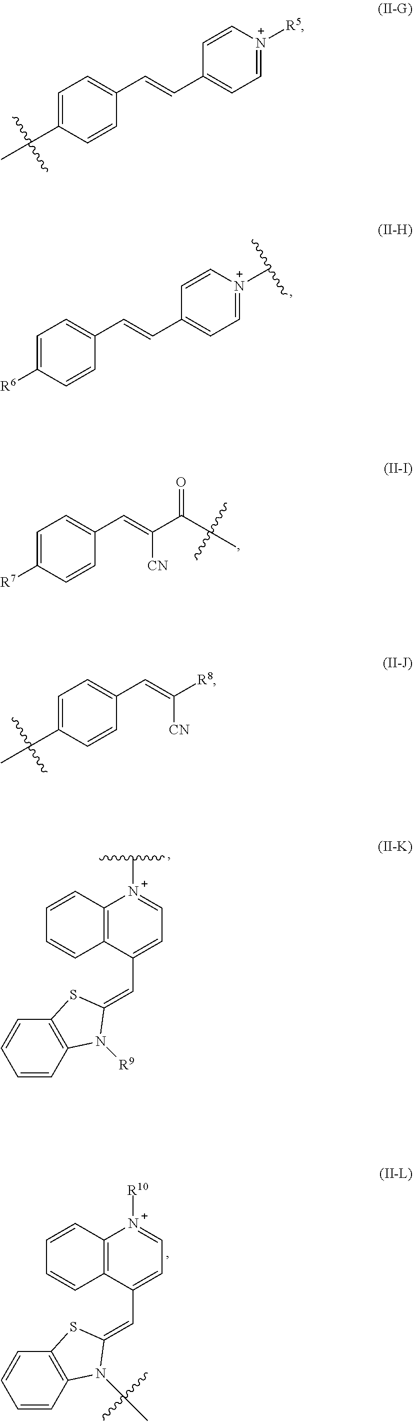
wherein:
X is selected from O or S;
X1 is O or NR⁴;
R¹, R³-R⁴ and R⁶-R⁹ are each independently selected from amino, substituted amino, alkyl, substituted alkyl, aryl, substituted aryl, acyl, substituted acyl, carboxyl, sulfonamide, substituted sulfonamide, nitro, nitrile, halogen, heteroaryl, substituted heteroaryl, heterocycle, and substituted heterocycle;
R² is selected from sulfonyl, amino, thiol and oxy;
R⁵ and R¹⁰ are independently selected from alkyl and substituted alkyl; and [symbol]
represents the point of attachment to L.

6. The probe of claim 1, wherein the linker comprises an alkyl chain, wherein at least one of the carbon atoms of the linker backbone is optionally substituted with a sulfur, nitrogen or oxygen heteroatom.

7. The probe of claim 6, wherein the linker additionally comprises a poly(ethylene glycol unit).

8. The probe of claim 1, wherein the linker is described by any one of formulae (LI)-(LV):
*—NR¹¹(CR¹²₂)ₙ— (LI);
—(CR¹²₂)ₙ— (LII);
*—NR¹¹(CH₂CH₂O)ₘ(CR¹²₂)ₙ— (LIII);
*—X²(CR¹²₂)ₙ— (LIV);
*—X²(CH₂CH₂O)ₘ(CR¹²₂)ₙ— (LV);
wherein:
R¹¹ and R¹² are each independently selected from hydrogen, alkyl and substituted alkyl;
X² is O or S;
n and m are each independently an integer from 1 to 10; and
* represents the point of attachment to the fluorophore A.

9. The probe of claim 8, wherein the linker is of the formula (L1), R¹¹ is selected from hydrogen or methyl, each R¹² group is hydrogen, and n is 2.

10. The probe of claim 8, wherein the linker is of the formula (L3), R¹¹ is hydrogen or methyl, each R¹² group is hydrogen, n is 2 and m is 1 or 2.

11. The probe of claim 1 wherein the compound is selected from the following structures:

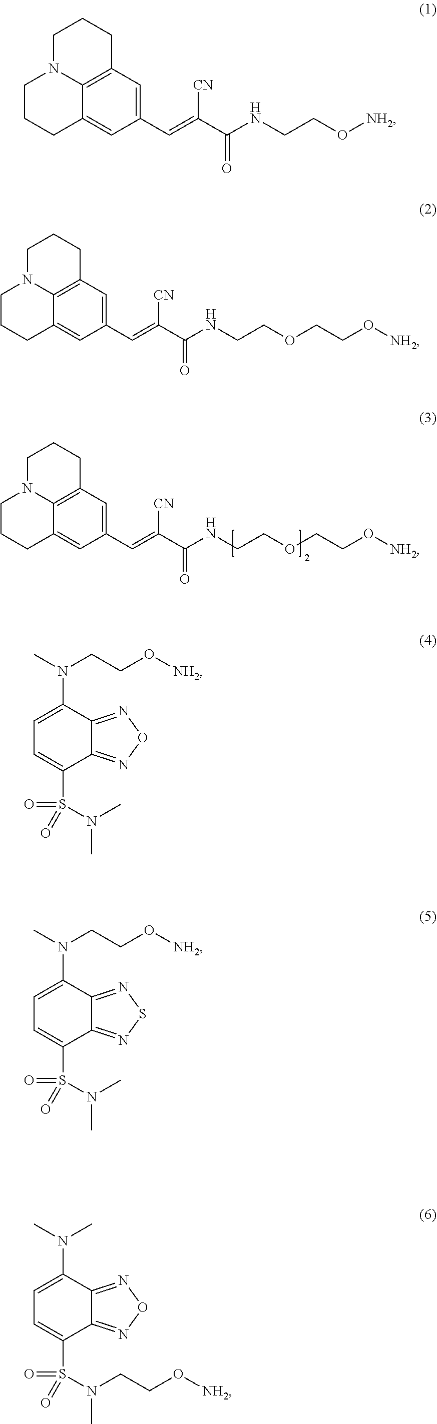

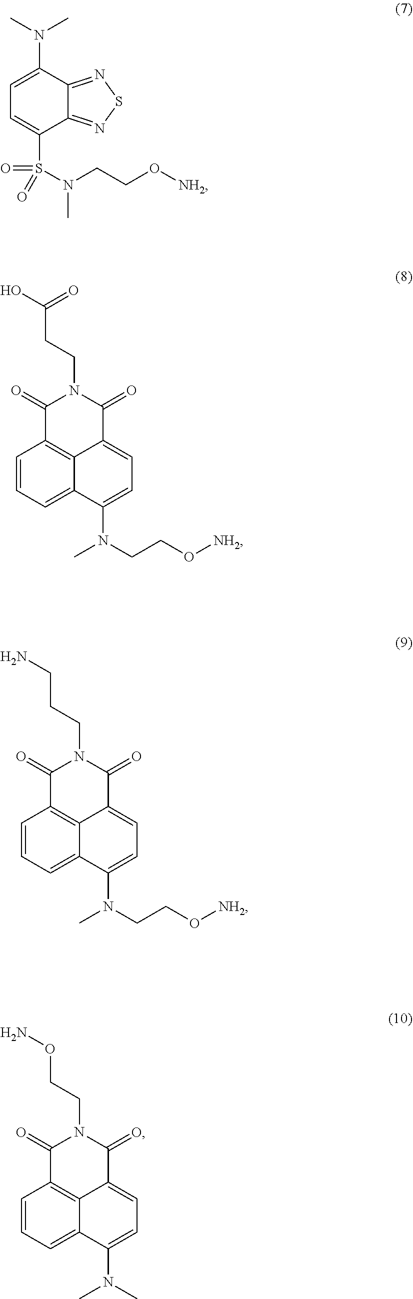 [SMILES: CN(C)c1ccc(S(=O)(=O)N(C)CCON)c2nsnc12 ; CN(CCON)c1ccc2c3c(cccc13)C(=O)N(CCC(=O)O)C2=O ; CN(CCON)c1ccc2c3c(cccc13)C(=O)N(CCCN)C2=O ; CN(C)c1ccc2c3c(cccc13)C(=O)N(CCON)C2=O]

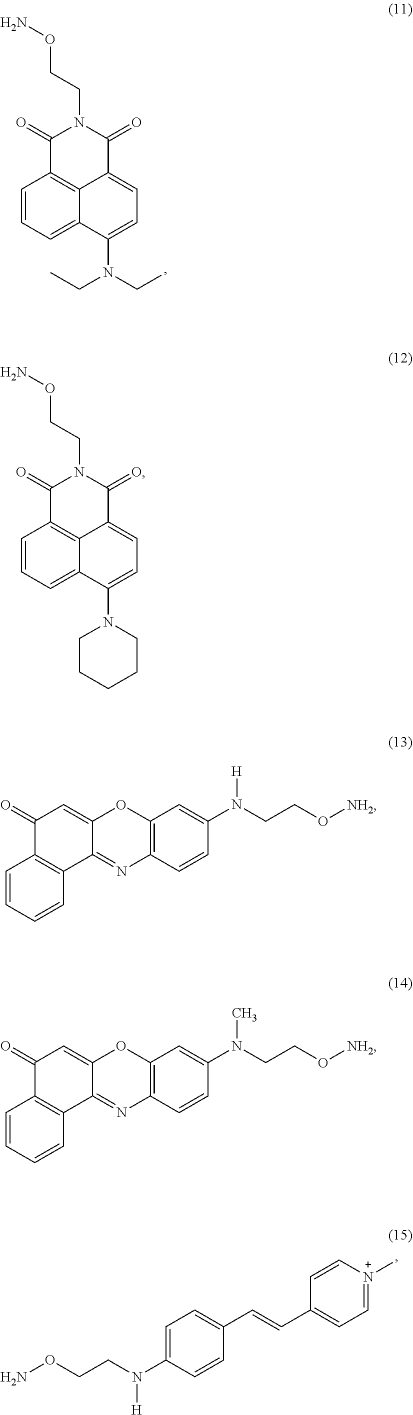

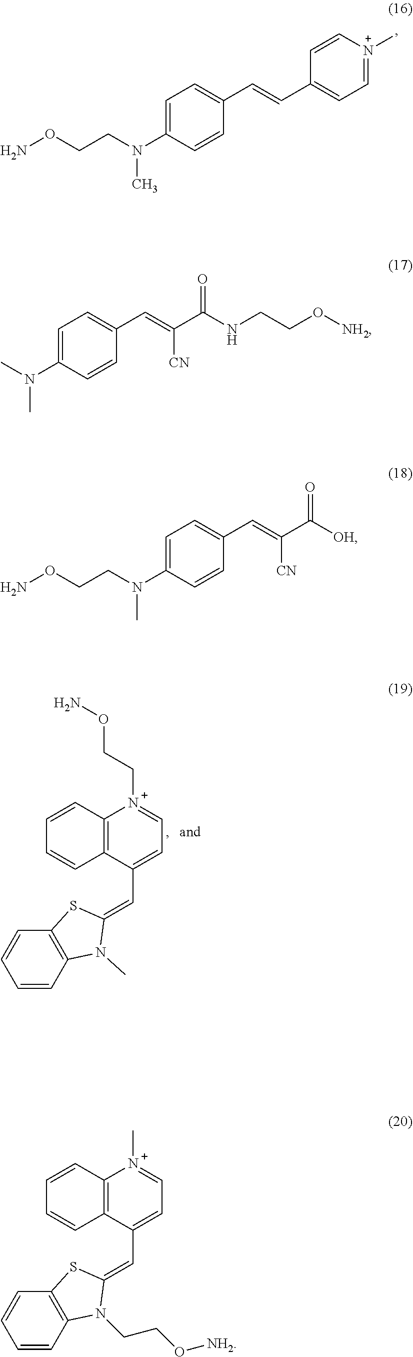

12. A method of detecting the presence of one or more abasic (AP) sites in a nucleic acid, the method comprising:
contacting the nucleic acid with a probe of any one of claims 1 to 11 under conditions for reaction of the alpha nucleophile of the probe with the AP sites in the nucleic acid thereby producing a conjugate; and
detecting a fluorescence response of the conjugate to determine the presence of one or more AP sites in the nucleic acid.

13. The method of claim 12, wherein the nucleic acid is DNA.

14. The method of claim 13, wherein the DNA is contacted with a glycosylase enzyme to generate DNA with AP sites, and the presence of one or more AP sites in the DNA is indicative of the glycosylase enzyme activity.

15. The method of claim 12, wherein the probe reacts selectively with the AP sites in the nucleic acid.

16. The method of claim 12, wherein the reaction to produce the conjugate has a reaction rate of at least 50 M⁻¹s⁻¹.

17. The method of any one of claim 12, wherein the fluorescence response of the conjugate is greater than that of the probe before contacting with the nucleic acid.

18. The method of claim 12, wherein the nucleic acid is a purified genomic DNA.

19. The method of claim 18, wherein the method further comprises comparing the fluorescence response of the conjugate to a standard to quantify the prevalence of AP sites in the purified genomic DNA.

20. The method of claim 17, further comprising pretreating the purified genomic DNA with a corresponding DNA repair enzyme before contacting with the probe, to generate a pre-treated DNA sample comprising AP sites.

21. (canceled)

22. A kit comprising:
a probe of any one of claims 1-11; and
a DNA repair enzyme.
